(19) United States
(12) Patent Application Publication
(10) Pub. No.: US 2007/0060629 A1
(43) Pub. Date: Mar. 15, 2007

(54) Large conductance calcium-activated k channel opener

(76) Inventors: Yasuhiro Imanishi, Osaka-shi (JP); Nobumasa Awai, Osaka-shi (JP); Miki Hirai, Osaka-shi (JP); Toshihiro Hosaka, Osaka-shi (JP); Rikako Kono, Osaka-shi (JP)

Correspondence Address: BIRCH STEWART KOLASCH & BIRCH, PO BOX 747, FALLS CHURCH, VA, 22040-0747, US

(21) Appl. No.: 10/574,529

(22) PCT Filed: Oct. 15, 2004
(86) PCT No.: PCT/JP04/15662

§ 371 Date: Apr. 4, 2006

Related U.S. Application Data
(60) Provisional application No. 60/584,451, filed on Jul. 1, 2004.

(30) Foreign Application Priority Data
Oct. 17, 2003 (JP) 2003-357325
Jan. 26, 2004 (JP) 2004-017662
Mar. 23, 2004 (JP) 2004-085143
Jun. 30, 2004 (JP) 2004-194172

(51) Int. Cl. C07D 231/12; C07D 261/06; A61K 31/42; A61K 31/415

(52) U.S. Cl. 514/378; 514/406; 548/247; 548/377.1

(57) ABSTRACT

The present invention provides a large conductance calcium-activated K channel opener comprising a compound of the formula (I): 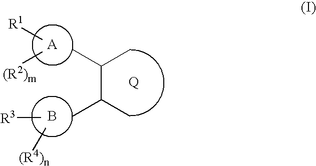
wherein R¹ and R³ are each sulfonamide, carbamoyl, acyl, amino, and the like, m and n are each 0 to 2, R² and R⁴ are each cyano, nitro, hydroxyl, an alkoxy, a halogen, or an alkyl, Ring A is benzene or a heterocyclic ring, Ring B is benzene, a heterocyclic ring, a cycloalkane etc, and Ring Q is pyrazole or isoxazole, or a pharmaceutically acceptable salt thereof as an active ingredient.

### FIELD OF THE INVENTION

This invention relates to a large conductance calcium-activated K channel opener, which is useful for treatment of disorders or diseases such as pollakiuria, urinary incontinence, asthma, chronic obstructive pulmonary diseases (COPD), cerebral infarction, subarachnoid hemorrhage, and the like.

### BACKGROUND OF THE INVENTION

Potassium is the most abundant intracelluar cation, and is very important in maintaining physiological homeostasis. Potassium channels are present in almost all vertebrate cells, and the potassium influx through these channels is indispensable for maintaining hyperpolarized resting membrane potential.

Large conductance calcium activated potassium channels (also BK channels or maxi-K channels) are expressed especially in neurons and smooth muscle cells. Because both of the increase of intracellular calcium concentration and membrane depolarization can activate maxi-K channels, maxi-K channels have been thought to play a pivotal role in regulating voltage-dependent calcium influx. Increase in the intracellular calcium concentration mediates many processes such as release of neurotransmitters, contraction of smooth muscles, cell growth and death, and the like. Actually, the opening of maxi-K channels causes strong membrane hyperpolarization, and inhibits these calcium-induced responses thereby. Accordingly, by inhibiting various depolarization-mediated physiological responses, a substance having an activity of opening maxi-K channels is useful for the treatment of diseases such as cerebral infarction, subarachnoid hemorrhage, pollakiuria, urinary incontinence, and the like.

There has been a report that a medicine which opens a BK channel has an activity to inhibit electrically induced contraction of respiratory tract preparation of guinea pig (J. Pharmacol. Exp. Ther., (1998) 286:952-958). Therefore, it is effective for treatment of, for example, asthma, COPD, etc. Also, there has been suggested that a medicine which opens a BK channel can be an agent for treatment of sexual function disorder such as erectile dysfunction, etc. (WO00/34244).

There have been various reports on a large conductance calcium-activated potassium channel opener. For example, a pyrrole derivative (e.g., WO96/40634), a furan derivative (e.g., JP2000-351773-A), a nitrogen-containing 5-membered ring derivative in which the nitrogen is substituted by phenyl or benzyl (e.g., WO98/04135), a diphenyltriazole derivative (e.g., J. Med. Chem., 2000, Vol. 45, p. 2942-2952), etc.

On the other hand, cycloxygenase 2 inhibitors such as Celecoxib, Valdecoxib, etc. have been used as a therapeutic agent for inflammation-related diseases such as chronic rheumatoid arthritis, etc., however, there have been no report regarding a use of these compounds for large conductance calcium-activated K channel opener (e.g., JP09-506350-A and JP09-500372-A).

Further, as a related compound, pyrazole derivatives have been known which are useful as a neurotensin receptor antagonist and a cycloxygenase inhibitor (e.g., JP11-504624-A, JP63-022080-A, J. Am. Chem. Soc., 1997, 119, 4882-4886, and J. Med. Chem., 1997, 40, 1347-1365).

### DISCLOSURE OF THE INVENTION

An object of the present invention is to provide a compound having an excellent large conductance calcium-activated K channel opening activity, and useful for the treatment of diseases such as pollakiuria, urinary incontinence, asthma, CPOD, cerebral infarction, subarachnoid hemorrhage, and the like.

The present inventors have studied intensively to solve the problem, and as a result, they have found that a compound of the formulae below has an excellent large conductance calcium-activated K channel-opening activity, whereby they have accomplished the present invention.

That is, the present inventions are as follows:

1. A large conductance calcium-activated K channel opener comprising a compound of the formula (I): 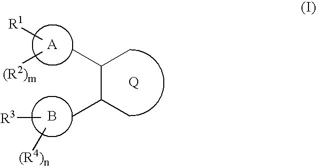
wherein Ring A is benzene or a heterocyclic ring;
Ring B is benzene, a heterocyclic ring, a cycloalkane or a cycloalkene;
Ring Q is a group selected from the following formulae: 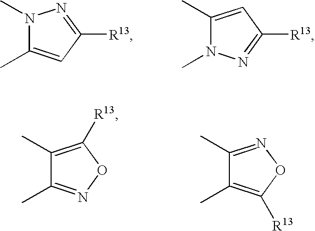
R¹ and R³ may be the same or different from each other, and each is a group selected from the following formulae: 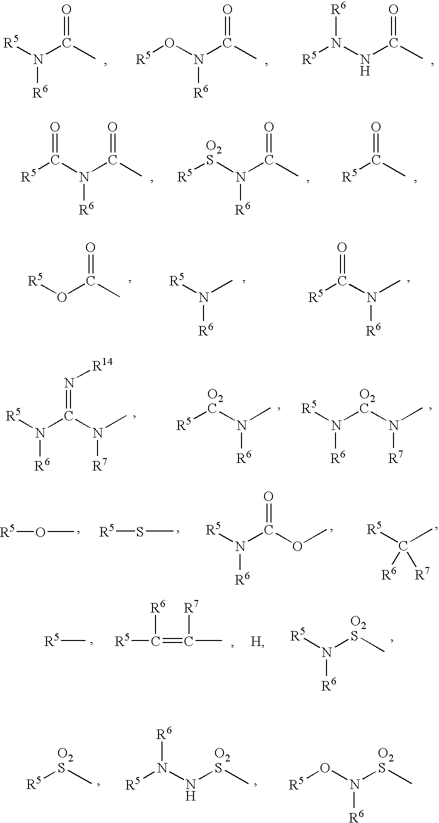
R⁵ and R⁶ may be the same or different from each other, and each is (1) hydrogen, (2) an optionally substituted alkyl, (3) an optionally substituted cycloalkyl which may be fused with an aryl, (4) an optionally substituted aryl, (5) an optionally substituted heterocyclic group, or (6) an alkoxycarbonyl, or (7) R⁵ and R⁶ may be combined to form an optionally substituted heterocyclic ring in combination with atom(s) to which they are bonded;
R⁷ is (1) hydrogen, (2) an optionally substituted alkyl, (3) an optionally substituted cycloalkyl which may be fused with an aryl, (4) an optionally substituted aryl, or (5) an alkoxycarbonyl;
R¹⁴ is hydrogen, an alkoxy, hydroxyl, cyano or an optionally substituted alkyl;
m and n may be the same or different from each other, and each is 0, 1 or 2;
R² and R⁴ may be the same or different from each other, and each is oxo, cyano, nitro, hydroxyl, an alkoxy, a halogen, carboxy, an alkoxycarbonyl, an optionally substituted carbamoyl, an optionally substituted amino or an optionally substituted alkyl; provided that when m is 2, two R² may be the same or different from each other, and when n is 2, two R⁴ may be the same or different from each other;
or R¹ and R² may be combined to form a group selected from the following formulae with Ring A; 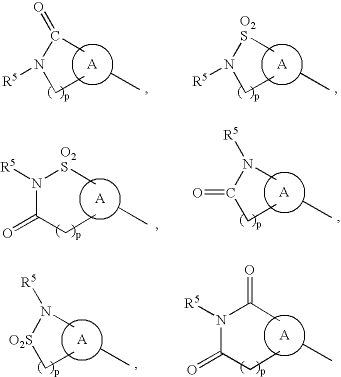
or R³ and R⁴ may be combined to form a group selected from the following formulae with Ring B; 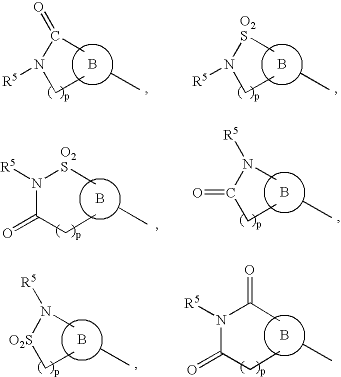
p is an integer of 1 to 3; and
R¹³ is (1) an optionally substituted alkyl, (2) cyano, (3) hydrogen, (4) a halogen, (5) an optionally substituted amino, (6) an alkenyl, (7) an optionally substituted carbamoyl, (8) an alkoxycarbonyl, (9) carboxy, (10) a heterocyclic group, (11) hydroxyl or (12) an alkoxy,
   or a pharmaceutically acceptable salt thereof as an active ingredient.

2. The large conductance calcium-activated K channel opener according to the above 1, wherein the substituent(s) for the optionally substituted alkyl of R⁵, R⁶ and R⁷ are 1 to 7 independently selected halogen(s) and/or 1 to 3 groups selected from the following groups: 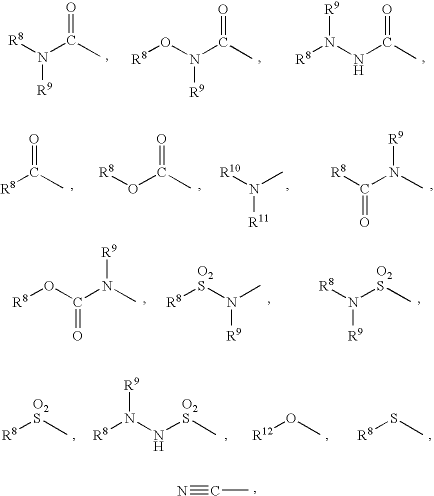
an optionally substituted heterocyclic group and an optionally substituted aryl,
wherein R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkoxycarbonyl, (6) an optionally substituted heterocyclic group or (7) an optionally substituted aryl, or (8) R⁸ and R⁹ may be combined to form an optionally substituted heterocyclic ring in combination with atom(s) to which they are bonded; R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group; R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group.

3. The large conductance calcium-activated K channel opener according to the above 1, wherein

Ring B is benzene, a heterocyclic ring, a cycloalkane or a cycloalkene,

R¹ is a group selected from the following formulae: 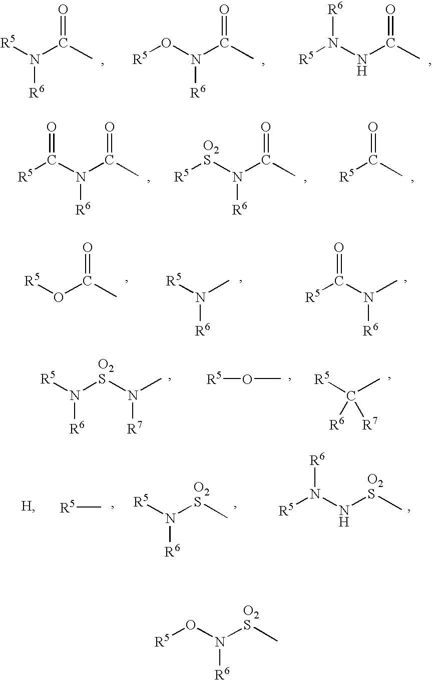
R³ is a group selected from the following formulae: 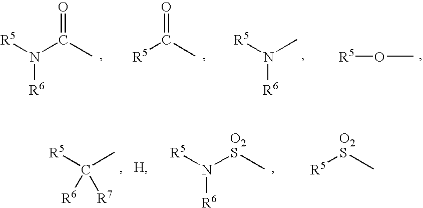
R⁵ is (1) hydrogen, (2) an alkyl which may be substituted by 1 to 7 independently selected halogen(s) and/or by 1 to 3 groups selected from the following groups: 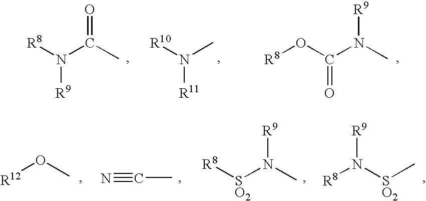
an optionally substituted heterocyclic group and an optionally substituted aryl,
   (3) an optionally substituted cycloalkyl which may be fused with an aryl, (4) an optionally substituted aryl, or (5) an optionally substituted heterocyclic group;
   R⁶ is hydrogen, an alkyl or an alkoxycarbonyl, or R⁵ and R⁶ may be combined to form an optionally substituted heterocyclic ring in combination with atom(s) to which they are bonded;
   R⁷ is hydrogen, an alkyl or an alkoxycarbonyl;
   R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, (6) an optionally substituted aryl, or (7) R⁸ and R⁹ may be combined to form an optionally substituted heterocyclic ring in combination with atoms to which they are bonded;
   R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group;
   R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
   m and n may be the same or different from each other, and each is 0, 1 or 2; and
   R² and R⁴ may be the same or different from each other, and each is oxo, cyano, nitro, hydroxyl, an alkoxy, a halogen or an optionally substituted alkyl.

4. The large conductance calcium-activated K channel opener according to the above 1, wherein

Ring B is (1) benzene or (2) a heterocyclic ring selected from thiophene, pyridine, pyrimidine, pyrazine, benzothiophene, 2,3-dihydroindole, 2,3-dihydrobenzofuran and 1,4-benzodioxane or (3) cyclohexene;

R¹ is a group selected from the following formulae: 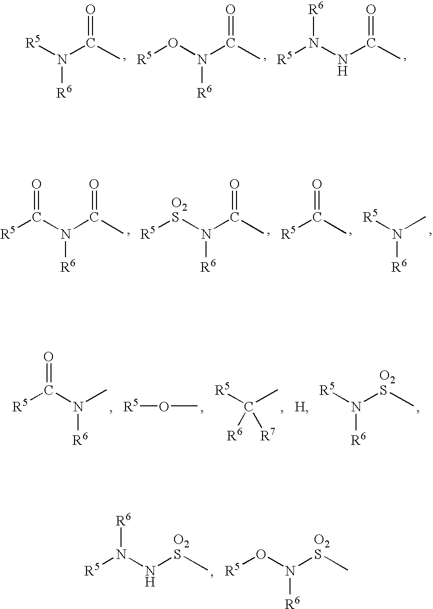
R³ is a group selected from the following formulae: 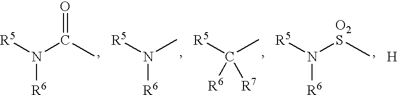
R⁵ is (1) hydrogen, (2) an alkyl which may be substituted by 1 to 7 independently selected halogen(s) and/or by 1 or 2 groups selected from the following groups: 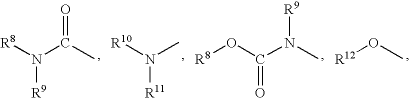
an optionally substituted heterocyclic group and an optionally substituted aryl,
   (3) an optionally substituted cycloalkyl which may be fused with an aryl, (4) an optionally substituted aryl, or (5) an optionally substituted heterocyclic group;
   R⁶ is hydrogen or an alkyl, or R⁵ and R⁶ may be combined to form a heterocyclic ring which may be substituted by hydroxyalkyl, in combination with atom(s) to which they are bonded;
   R⁷ is hydrogen or an alkyl;
   R⁸ and R⁹ may be the same or different from each other, and each is (1) hydrogen, (2) an alkyl which may be substituted by an optionally substituted aryl or by an optionally substituted heterocyclic group, (3) hydroxyalkyl or (4) an alkoxyalkyl;
   R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
   R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
   m and n may be the same or different from each other, and each is 0, 1 or 2;
   R² and R⁴ may be the same or different from each other, and each is oxo, cyano, nitro, hydroxyl, an alkoxy, a halogen organ alkyl which may be substituted by hydroxyl(s); and
   R¹³ is (1) hydrogen, (2) an alkyl which may be substituted by group(s) selected from a halogen, hydroxyl, an optionally substituted alkoxy, cyano, carboxy, carbamoyl, an alkoxycarbonyl, an optionally substituted amino and an optionally substituted imino, (3) an alkenyl, or (4) a heterocyclic group.

5. The large conductance calcium-activated K channel opener according to the above 1, wherein

Ring A is benzene, thiophene, pyridine or pyrazole;

Ring B is (1) benzene, (2) a heterocyclic ring selected from thiophene, pyridine, pyrimidine, pyrazine, benzothiophene, 2,3-dihydroindole and 1,4-benzodioxane, or (3) cyclohexene;

R¹ is a group selected from the following formulae: 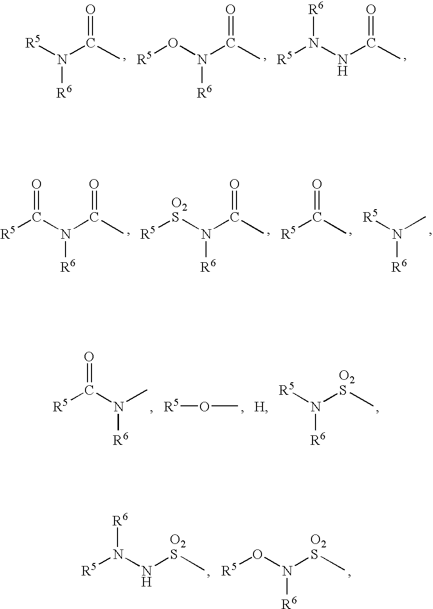
R³ is a group selected from the following formulae: 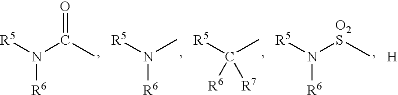
R⁵ is (1) hydrogen, (2) an alkyl which may be substituted by 1 to 7 independently selected halogen(s) and/or by 1 or 2 groups selected from the following groups: 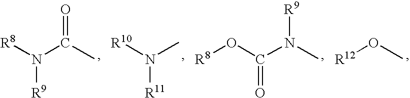
an optionally substituted heterocyclic group and an optionally substituted aryl,
   (3) a cycloalkyl fused with an aryl which may be substituted by hydroxyl(s), or (4) a heterocyclic group;
   R⁶ is hydrogen or an alkyl, or R⁵ and R⁶ may be combined to form a heterocyclic ring which may be substituted by hydroxyalkyl;
   R⁷ is hydrogen or an alkyl;
   R⁸, R⁹, R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, or (6) an optionally substituted aryl;
   R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
   m and n may be the same or different from each other, and each is 0, 1 or 2;
   R² and R⁴ may be the same or different from each other, and each is cyano, nitro, hydroxyl, a halogen, an alkyl or an alkoxy; and
   R¹³ is (1) hydrogen, (2) an alkyl which may be substituted by group(s) selected from a halogen, hydroxyl, an alkoxy which may be substituted by group(s) selected from a halogen and phenyl, cyano, carboxy, carbamoyl, an alkoxycarbonyl, an amino which may be substituted by phenyl, and an imino which may be substituted by group(s) selected from an alkoxy and hydroxyl, (3) an alkenyl or (4) 4,5-dihydroxazol-2-yl.

6. The large conductance calcium-activated K channel opener according to any one of the above 1 to 5, wherein R¹ is a group selected from the following formulae:

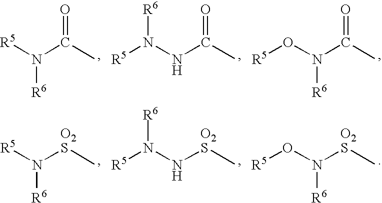

7. A compound of the formula (Ia): 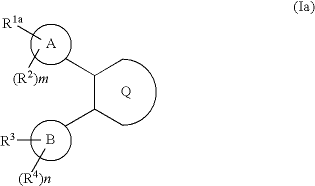
wherein Ring A is benzene or a heterocyclic ring;
Ring B is benzene, a heterocyclic ring, a cycloalkane or a cycloalkene;
Ring Q is a group selected from the following formulae: 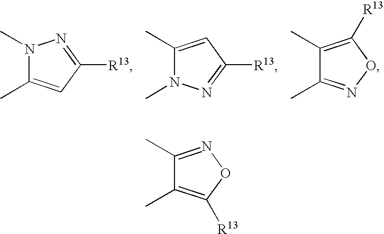
R^{1a} is a group selected from the following formulae: 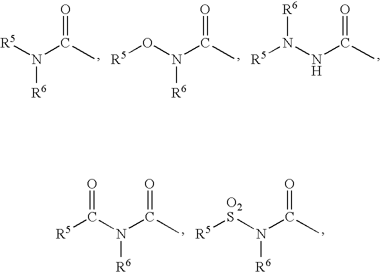
R³ is a group selected from the following formulae: 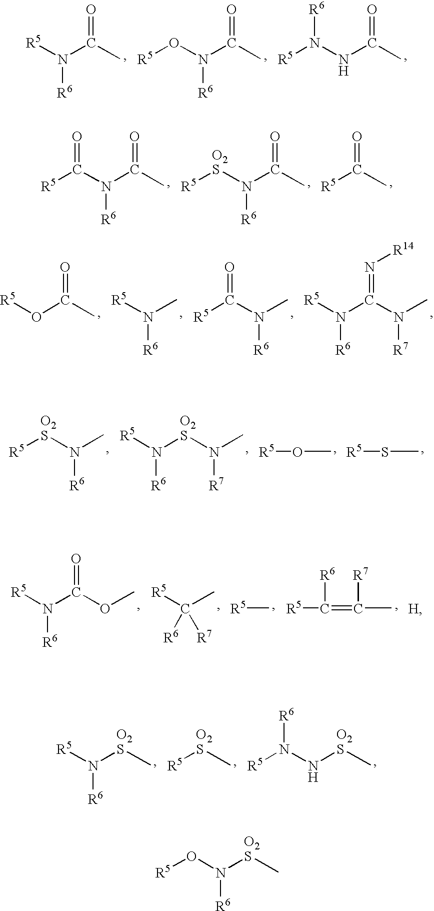
R⁵ and R⁶ may be the same or different from each other, and each is (1) hydrogen, (2) an optionally substituted alkyl, (3) an optionally substituted cycloalkyl which may be fused with an aryl, (4) an optionally substituted aryl, (5) an optionally substituted heterocyclic group, or (6) an alkoxycarbonyl, or (7) R⁵ and R⁶ may be combined to form an optionally substituted heterocyclic ring in combination with atom(s) to which they are bonded;
R⁷ is (1) hydrogen, (2) an optionally substituted alkyl, (3) an optionally substituted cycloalkyl which may be fused with an aryl, (4) an optionally substituted aryl, or (5) an alkoxycarbonyl;
R¹⁴ is hydrogen, an alkoxy, hydroxyl, cyano or an optionally substituted alkyl;
m and n may be the same or different from each other, and each is 0, 1 or 2;
R² and R⁴ may be the same or different from each other, and each is oxo, cyano, nitro, hydroxyl, an alkoxy, a halogen, carboxy, an alkoxycarbonyl, a carbamoyl which may be substituted, an amino which may be substituted or an alkyl which may be substituted; provided that when m is 2, two R² may be the same or different from each other, and when n is 2, two R⁴ may be the same or different from each other; or R^{1a} and R² may be combined to form a group of the following formula with Ring A: 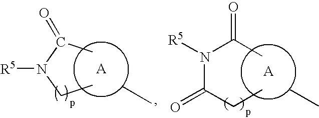
or R³ and R⁴ may be combined to form a group selected from the following formulae with Ring B: 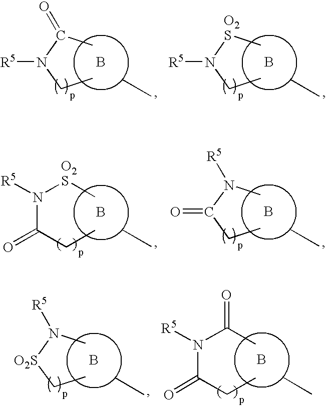
p is an integer of 1 to 3; and
R¹³ is (1) an optionally substituted alkyl, (2) cyano, (3) hydrogen, (4) a halogen, (5) an optionally substituted amino, (6) an alkenyl, (7) an optionally substituted carbamoyl, (8) an alkoxycarbonyl, (9) carboxy, (10) a heterocyclic group, (11) hydroxyl or (12) an alkoxy;
provided that (i) the compound wherein Ring A and
Ring B are benzenes;
Ring Q is 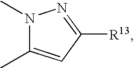
R³ is hydroxyl, an alkoxy or a cycloalkyloxy which are substituted at 2-position,
R⁴ is methoxy substituted at 6-position, and
R¹³ is an alkoxycarbonyl or carboxy,
(ii) N-(3-isopropoxypropyl)-4-(3-methyl-5-phenyl-1H-pyrazol-1-yl)benzamide,
(iii) 4-(1-(4-aminosulfonylphenyl)-3-difluoromethyl-1H-pyrazol-5-yl)benzamide, and
(iv) 4-[5-(4-chlorophenyl)-3-(3-hydroxypropyl)-1H-pyrazol-1-yl]-N-methylbenzohydroxamic acid are excluded,
   or a pharmaceutically acceptable salt thereof.

8. The compound or a pharmaceutically acceptable salt thereof according to the above 7, wherein the substituent(s) for the optionally substituted alkyl of R⁵, R⁶ and R⁷ are 1 to 7 independently selected halogen(s) and/or 1 to 3 groups selected from the following groups: 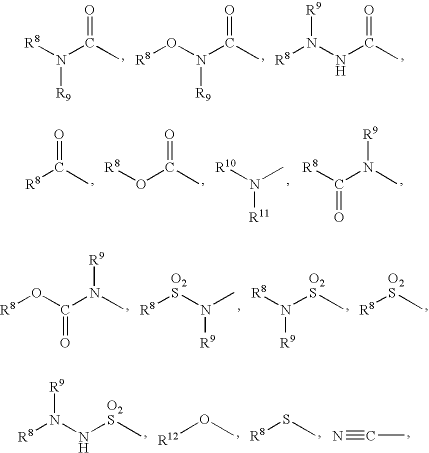
an optionally substituted heterocyclic group and an optionally substituted aryl,
wherein R⁸ and R⁹ may be the same or different from, each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkoxycarbonyl, (6) an optionally substituted heterocyclic group or (7) an optionally substituted aryl, or (8) R⁸ and R⁹ may be combined to form an optionally substituted heterocyclic ring in combination with atom(s) to which they are bonded; R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group; R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group.

9. The compound or a pharmaceutically acceptable salt thereof according to the above 7, wherein

Ring B is benzene, a heterocyclic ring or a cycloalkane;

R^{1a} is a group selected from the following formulae: 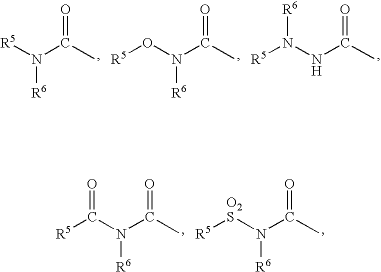
R³ is a group selected from the following formulae: 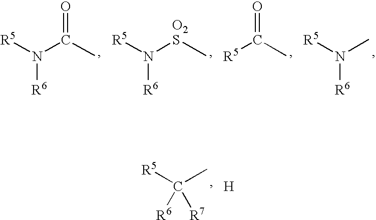
R⁵ is (1) hydrogen, (2) an alkyl which may be substituted by 1 to 7 independently selected halogen(s) and/or by 1 to 3 groups selected from the following groups: 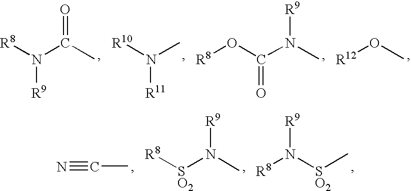
an optionally substituted heterocyclic group and an optionally substituted aryl,
(3) an optionally substituted cycloalkyl which may be fused with an aryl, (4) an optionally substituted aryl, or (5) an optionally substituted heterocyclic group;
R⁶ is hydrogen or an alkyl, or R⁵ and R⁶ may be combined to form an optionally substituted heterocyclic ring in combination with atom(s) to which they are bonded;
R⁷ is hydrogen, an alkyl or an alkoxycarbonyl;
R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, (6) an optionally substituted aryl, or (7) R⁸ and R⁹ may be combined to form an optionally substituted heterocyclic ring in combination with atom(s) to which they are bonded;
R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group;
R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
m and n may be the same or different from each other, and each is 0, 1 or 2; and
R² and R⁴ may be the same or different from each other, and each is oxo, cyano, nitro, hydroxyl, an alkoxy, a halogen or an optionally substituted alkyl.

10. The compound or a pharmaceutically acceptable salt thereof according to the above 7, wherein

Ring B is (1) benzene or (2) a heterocyclic ring selected from thiophene, pyridine, pyrimidine, pyrazine, benzothiophene, 2,3-dihydroindole, 2,3-dihydrobenzofuran and 1,4-benzodioxane;

R^{1a} is a group selected from the following formulae: 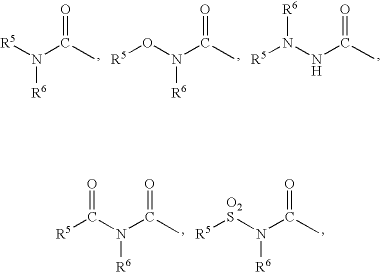
R³ is a group selected from the following formulae: 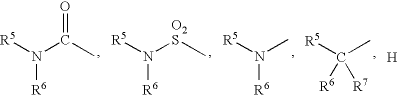
R⁵ is (1) hydrogen, (2) an alkyl which may be substituted by 1 to 7 independently selected halogen(s) and/or by 1 or 2 groups selected from the following groups: 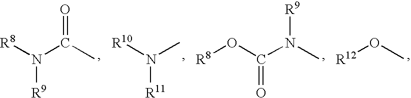
an optionally substituted heterocyclic group and an optionally substituted aryl,
   (3) an optionally substituted cycloalkyl which may be fused with an aryl, (4) an optionally substituted aryl, or (5) an optionally substituted heterocyclic group;
   R⁶ is hydrogen or an alkyl, or R⁵ and R⁶ may be combined to form a heterocyclic ring which may be substituted by a hydroxyalkyl, in combination with atom(s) to which they are bonded;
   R⁷ is hydrogen or an alkyl;
   R⁸ and R⁹ may be the same or different from each other, and each is (1) hydrogen, (2) an alkyl which may be substituted by an optionally substituted aryl or by an optionally substituted heterocyclic group, (3) a hydroxyalkyl or (4) an alkoxyalkyl;
   R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
   R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
   m and n may be the same or different from each other, and each is 0, 1 or 2;
   R² and R⁴ may be the same or different from each other, and each is oxo, cyano, nitro, hydroxyl, an alkoxy, a halogen or an alkyl which may be substituted by hydroxyl(s); and
   R¹³ is (1) hydrogen, (2) an alkyl which may be substituted by group(S) selected from a halogen, hydroxyl, an optionally substituted alkoxy, cyano, carboxy, an optionally substituted amino and an optionally substituted imino, (3) an alkenyl, or (4) a heterocyclic group.

11. The compound or a pharmaceutically acceptable salt thereof according to the above 7, wherein

Ring A is benzene, thiophene, pyridine or pyrazole;

Ring B is (1) benzene, or (2) a heterocyclic ring selected from thiophene, pyridine, pyrimidine, pyrazine, benzothiophene and 1,4-benzodioxane;

R^{1a} is a group selected from the following formulae: 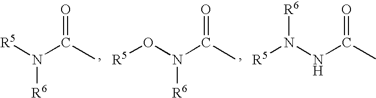
R³ is a group selected from the following formulae: 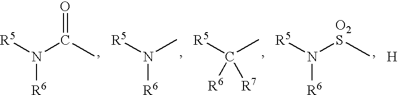
R⁵ is (1) hydrogen, (2) an alkyl which may be substituted by 1 to 7 independently selected halogen(s) and/or by 1 or 2 groups selected from the following groups: 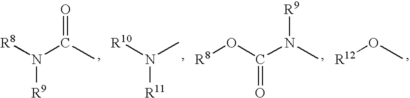
an optionally substituted heterocyclic group and an optionally substituted aryl,
   (3) a cycloalkyl fused with an aryl which may be substituted by hydroxyl, or (4) a heterocyclic group;
   R⁶ is hydrogen or an alkyl, or R⁵ and R⁶ may be combined to form a heterocyclic ring which may be substituted by hydroxyalkyl;
   R⁷ is hydrogen or an alkyl;
   R⁸, R⁹, R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, or (6) an optionally substituted aryl;
   R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
   m and n may be the same or different from each other, and each is 0, 1 or 2;
   R² and R⁴ may be the same or different from each other, and each is cyano, nitro, hydroxyl, a halogen, an alkyl or an alkoxy; and
   R¹³ is (1) hydrogen, (2) an alkyl which may be substituted by group(s) selected from a halogen, hydroxyl, an alkoxy which may be substituted by group(s) selected from a halogen and phenyl, cyano, carboxy, carbamoyl, an alkoxycarbonyl, an amino which may be substituted by phenyl, and an imino which may be substituted by group(s) selected from an alkoxy and hydroxyl, (3) an alkenyl or (4) 4,5-dihydroxazol-2-yl.

12. A medicine comprising the compound or a pharmaceutically acceptable salt thereof according to any one of the above 7 to 11.

13. The medicine according to the above 12, which is a large conductance calcium-activated K channel opener.

14. The large conductance calcium-activated K channel opener according to any one of the above 1 to 5 and 13, which is for the prophylaxis and/or treatment of pollakiuria, urinary incontinence, asthma or COPD.

15. A compound of the formula (I): 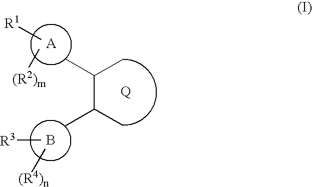
wherein Ring A is benzene or a heterocyclic ring;
Ring B is benzene, a heterocyclic ring, a cycloalkane or a cycloalkene;
Ring Q is a group selected from the following formulae: 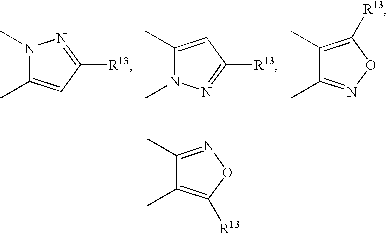
R¹ and R³ may be the same or different from each other, and each is a group selected from the following formulae: 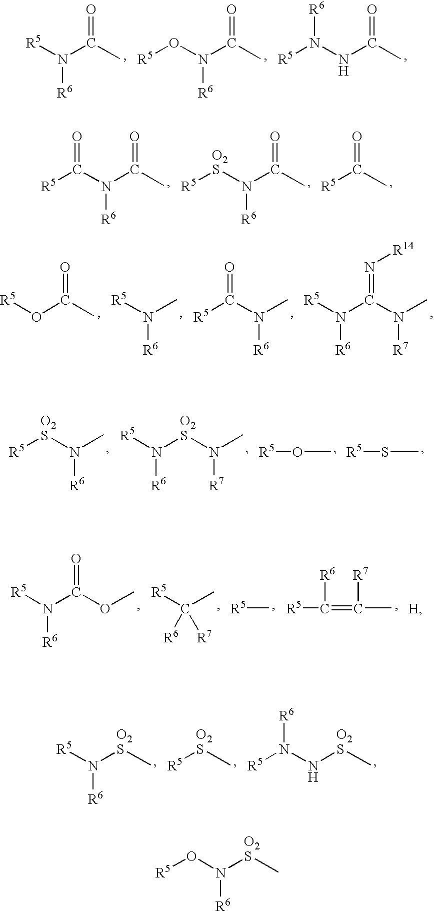
R⁵ and R⁶ may be the same or different from each other, and each is (1) hydrogen, (2) an optionally substituted alkyl, (3) an optionally substituted cycloalkyl which may be fused with an aryl, (4) an optionally substituted aryl, (5) an optionally substituted heterocyclic group, or (6) an alkoxycarbonyl, or (7) R⁵ and R⁶ may be combined to form an optionally substituted heterocyclic ring in combination with atom(s) to which they are bonded;
R⁷ is (1) hydrogen, (2) an optionally substituted alkyl, (3) an optionally substituted cycloalkyl which may be fused with an aryl, (4) an optionally substituted aryl, or (5) an alkoxycarbonyl;
R¹⁴ is hydrogen, an alkoxy, hydroxyl, cyano or an optionally substituted alkyl;
m and n may be the same or different from each other, and each is 0, 1 or 2;
R² and R⁴ may be the same or different from each other, and each is oxo, cyano, nitro, hydroxyl, an alkoxy, a halogen, carboxy, an alkoxycarbonyl, an optionally substituted carbamoyl, an optionally substituted amino or an optionally substituted alkyl; provided that when m is 2, two R²s may be the same or different from each other, and when n is 2, two R⁴s may be the same or different from each other;
or R¹ and R² may be combined to form a group selected from the following formulae with Ring A; 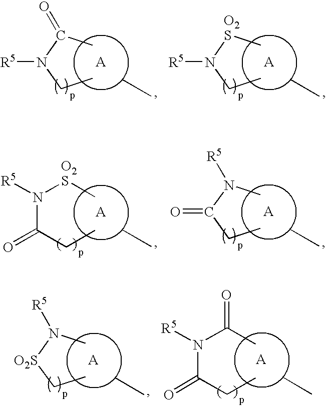
or R³ and R⁴ may be combined to form a group selected from the following formulae with Ring B; 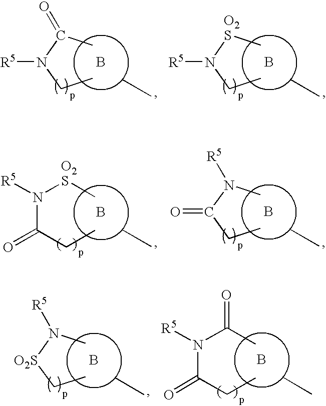
p is an integer of 1 to 3; and
R¹³ is (1) an optionally substituted alkyl, (2) cyano, (3) hydrogen, (4) a halogen, (5) an optionally substituted amino, (6) an alkenyl, (7) an optionally substituted carbamoyl, (8) an alkoxycarbonyl, (9) carboxy, (10) a heterocyclic group, (11) hydroxyl or (12) an alkoxy;
provided that the compound wherein Ring A is benzene;
Ring B is benzene, pyridine or a cycloalkane;
Ring Q is 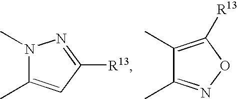
wherein R¹³ is a halogen, an alkyl or a haloalkyl;
R¹ is sulfamoyl or an alkylsulfonyl;
R³ is hydrogen, an alkyl or an alkoxy; and
when m is 1, R² is a halogen; or m is 0; and
when n is 1, R⁴ is a halogen, an alkoxy or an alkyl;

or n is 0
is excluded,
   or a pharmaceutically acceptable salt thereof.

16. The compound or a pharmaceutically acceptable salt thereof according to the above 15, wherein the substituent(s) for the optionally substituted alkyl of R⁵, R⁶ and R⁷ are 1 to 7 independently selected halogen(s) and/or 1 to 3 groups selected from the following groups: 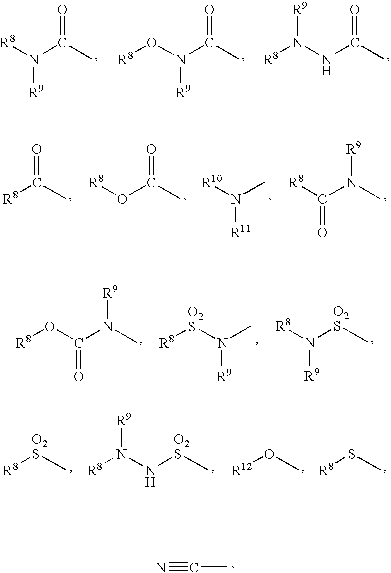
an optionally substituted heterocyclic group and an optionally substituted aryl,
   wherein R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkoxycarbonyl, (6) an optionally substituted heterocyclic group or (7) an optionally substituted aryl, or (8) R⁸ and R⁹ may be combined to form an optionally substituted heterocyclic ring in combination with atom(s) to which they are bonded; R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group; R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group.

17. A large conductance calcium-activated K channel opener comprising a compound of the formula (I) or a pharmaceutically acceptable salt thereof according to the above 15 or 16.

18. The large conductance calcium-activated K channel opener according to any one of above 1 to 5 and 17, wherein neither R¹ nor R³ is hydrogen.

19. The compound according to the above 15 or 16, wherein neither R¹ nor R³ is hydrogen, or a pharmaceutically acceptable salt thereof.

20. A large conductance calcium-activated K channel opener, comprising a compound of the formula: 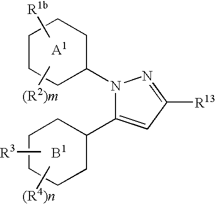
wherein Ring A¹ and Ring B¹ may be the same or different from each other, and each is benzene, pyridine, a cyclohexane, or a cyclohexene; R^{1b} is a group selected from the following formulae: 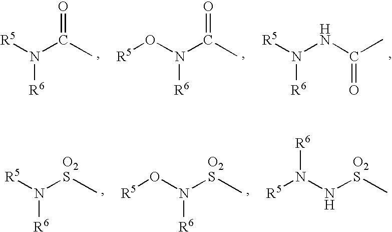
and the other symbols have the same meanings as defined above;
provided that R^{1b} is preferably bonded at m- or p-position of Ring A¹, more preferably at p-position of Ring A¹ and that Ring A¹ and Ring B¹ are each preferably benzene or pyridine;
   or a pharmaceutically acceptable salt thereof, as an active ingredient.

21. A large conductance calcium-activated K channel opener, comprising a compound of the formula: 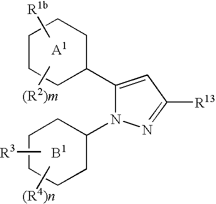
wherein each symbol has the same meaning as defined above;
provided that R^{1b} is preferably bonded at m- or p-position of Ring A¹, more preferably at p-position of Ring A¹ and that Ring A¹ and Ring B¹ are each preferably benzene or pyridine;
   or a pharmaceutically acceptable salt thereof, as an active ingredient.

22. A large conductance calcium-activated K channel opener, comprising a compound of the formula: 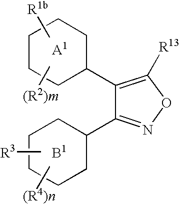
wherein each symbol has the same meaning as defined above;
provided that R^{1b} is preferably bonded at m- or p-position of Ring A¹, more preferably at p-position of Ring A¹ and that Ring A¹ and Ring B¹ are each preferably benzene or pyridine;
   or a pharmaceutically acceptable salt thereof, as an active ingredient.

23. A large conductance calcium-activated K channel opener, comprising a compound of the formula: 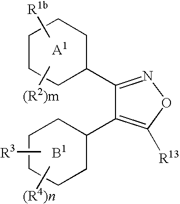
wherein each symbol has the same meaning as defined above;
provided that R^{1b} is preferably bonded at m- or p-position of Ring A¹, more preferably at p-position of Ring A¹ and that Ring A¹ and Ring B¹ are each preferably benzene or pyridine;
   or a pharmaceutically acceptable salt thereof, as an active ingredient:

24. A compound of the formula: 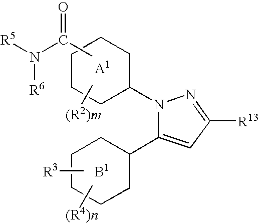
wherein each symbol has the same meaning as defined above;
provided that R⁵R⁶NCO— is preferably bonded at m- or p-position of Ring A¹, more preferably at p-position of Ring A¹ and that Ring A¹ and Ring B¹ are each preferably benzene or pyridine;
   or a pharmaceutically acceptable salt thereof.

25. A compound of the formula: 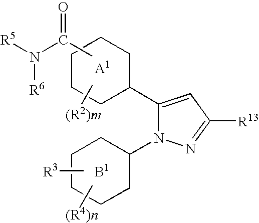
wherein each symbol has the same meaning as defined above;
provided that R⁵R⁶NCO— is preferably bonded at m- or p-position of Ring A¹, more preferably at p-position of Ring A¹ and that Ring A¹ and Ring B¹ are each preferably benzene or pyridine;
   or a pharmaceutically acceptable salt thereof.

26. A compound of the formula: 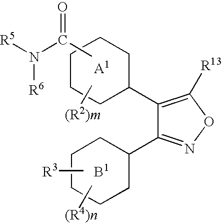
wherein each symbol has the same meaning as defined above;
provided that R⁵R⁶NCO— is preferably bonded at m- or p-position of Ring A¹, more preferably at p-position of Ring A¹ and that Ring A¹ and Ring B¹ are each preferably benzene or pyridine;
   or a pharmaceutically acceptable salt thereof.

27. A compound of the formula: 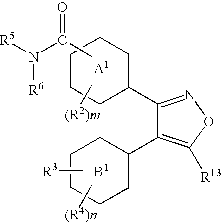
wherein each symbol has the same meaning as defined above;
provided that R⁵R⁶NCO— is preferably bonded at m- or p-position of Ring A¹, more preferably at p-position of Ring A¹ and that Ring A¹ and Ring B¹ are each preferably benzene or pyridine;
   or a pharmaceutically acceptable salt thereof.

28. The large conductance calcium-activated K channel opener, according to any one of the above 20 to 23, wherein R³ is a group selected from the following formulae:

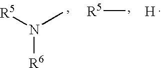

29. The compound or a pharmaceutically acceptable salt thereof according to any one of the above 24 to 27, wherein R³ is a group selected from the following formulae:

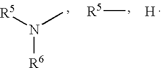

30. The large conductance calcium-activated K channel opener according to any one of the above 20 to 23, wherein R⁵ is an alkyl which may be substituted by 1 to 7 independently selected halogen(s) and/or 1 to 3 groups selected from the following groups: 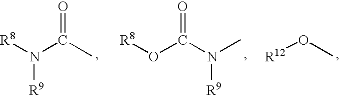
and an optionally substituted heterocyclic group.

31. The compound or a pharmaceutically acceptable salt thereof according to any one of the above 24 to 27, wherein R⁵ is an alkyl which may be substituted by 1 to 7 independently selected halogen(s) and/or 1 to 3 groups selected from the following groups: 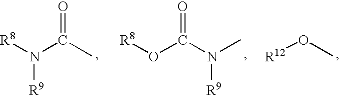
and an optionally substituted heterocyclic group.

32. A compound of the formula: 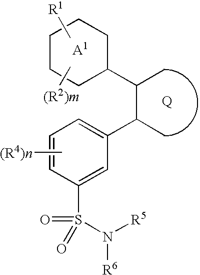
wherein each symbol has the same meaning as defined in the above 1;
provided that Ring A¹ is preferably benzene or pyridine, and Ring Q is preferably 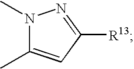
   or a pharmaceutically acceptable salt thereof.

33. The compound or a pharmaceutically acceptable salt thereof according to the above 32, wherein Ring A¹ is benzene or pyridine.

34. The compound or a pharmaceutically acceptable salt thereof according to the above 32 or 33, wherein R¹ is hydrogen or methyl, m is 0, R⁴ is methyl, and n is 1.

35. A large conductance calcium-activated K channel opener, comprising a compound of the formula: 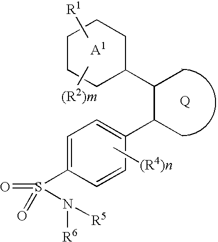
wherein each symbol has the same meaning as defined above;
provided that Ring A¹ is preferably-benzene or pyridine, and Ring Q is preferably 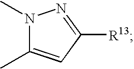
   or a pharmaceutically acceptable salt thereof, as an active ingredient.

36. A compound of the formula: 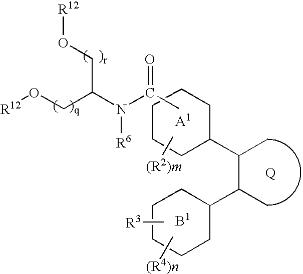
wherein q and r are each an integer of 1 to 6, and the other symbols have the same meanings as defined above;
provided that the group [R¹²O(CH₂)_{q}][R¹²O(CH₂)ᵣ]CHN(R⁶)CO— is preferably bonded at m- or p-position of Ring A¹, more preferably at p-position of Ring A¹, two R¹²s may be the same or different from each other, and Ring A¹ and Ring B¹ are each preferably benzene or pyridine;
   or a pharmaceutically acceptable salt thereof.

37. A compound of the formula: 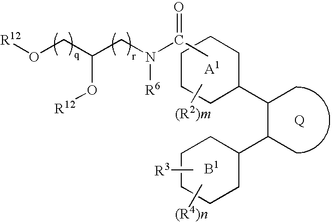
wherein each symbol has the same meanings as defined above;
provided that the group [R¹²O(CH₂)_{q}](R¹²O)CH(CH₂)ᵣN(R⁶)CO— is preferably bonded at m- or p-position of Ring A¹, more preferably at p-position of Ring A¹, two R¹²s may be the same or different from each other, and Ring A¹ and Ring B¹ are each preferably benzene or pyridine;
   or a pharmaceutically acceptable salt thereof.

38. A compound of the formula: 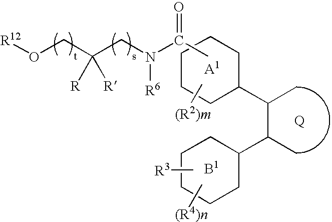
wherein s and t are each an integer of 0 to 6, R and R′ are each hydrogen or an alkyl, and the other symbols have the same meanings as defined above;
provided that the group R¹²O(CH₂)ₜC(R)(R′)(CH₂)ₛN(R⁶)CO— is preferably bonded at m- or p-position of Ring A¹, more preferably at p-position of Ring A¹ and Ring A¹ and Ring B¹ are each preferably benzene or pyridine;
   or a pharmaceutically acceptable salt thereof.

39. A compound of the formula: 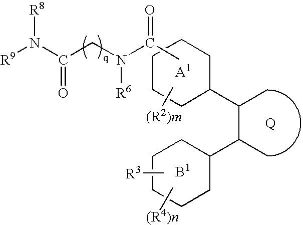
wherein each symbol has the same meanings as defined above;
provided that the group R⁹R⁸NCO—(CH₂)_{q}N(R⁶)CO— is preferably bonded at m- or p-position of Ring A¹, more preferably at p-position of Ring A¹ and Ring A¹ and Ring B¹ are each preferably benzene or pyridine;
   or a pharmaceutically acceptable salt thereof.

40. A compound of the formula: 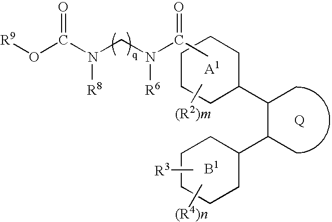
wherein each symbol has the same meanings as defined above;
provided that the group R⁹OCON(R⁸)(CH₂)_{q}N(R⁶)CO— is preferably bonded at m- or p-position of Ring A¹, more preferably at p-position of Ring A¹ and Ring A¹ and Ring B¹ are each preferably benzene or pyridine;
   or a pharmaceutically acceptable salt thereof.

41. A compound of the formula: 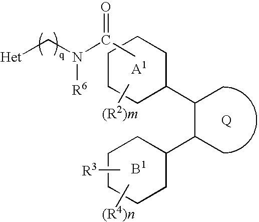
wherein Het is an optionally substituted heterocyclic group, and the other symbols have the same meanings as defined above;
provided that the group Het(CH₂)_{q}N(R⁶)CO— is preferably bonded at m- or p-position of Ring A¹, more preferably at p-position of Ring A¹ and Ring A¹ and Ring B¹ are each preferably benzene or pyridine;
   or a pharmaceutically acceptable salt thereof.

42. A compound of the formula: 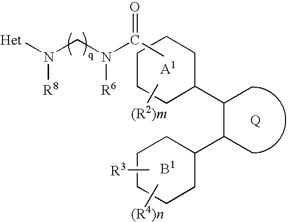
wherein each symbol has the same meanings as defined above;
provided that the group HetN(R⁸)(CH₂)_{q}N(R⁶)CO— is preferably bonded at m- or p-position of Ring A¹, more preferably at p-position of Ring A¹ and Ring A¹ and Ring B¹ are each preferably benzene or pyridine;
   or a pharmaceutically acceptable salt thereof.

43. A compound of the formula: 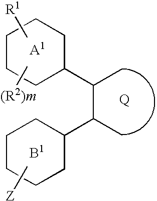
wherein Z is an alkyl, a halogen or an optionally substituted amino, and the other symbols have the same meanings as defined above; provided that R¹ is preferably bonded at m- or p-position of Ring A¹, more preferably at p-position of Ring A¹ and Ring A¹ and Ring B¹ are each preferably benzene or pyridine;
   or a pharmaceutically acceptable salt thereof.

44. The compound or a pharmaceutically acceptable salt thereof according to the above 43, wherein R¹ is a group selected from the following formulae: 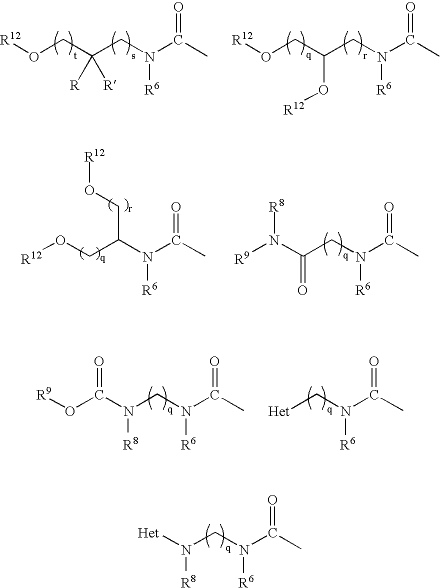
wherein each symbol has the same meaning as defined above.

45. A compound of the formula (I-1): 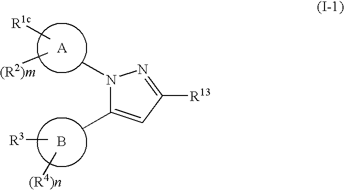
wherein R^{1c} is a group selected from the following formulae: 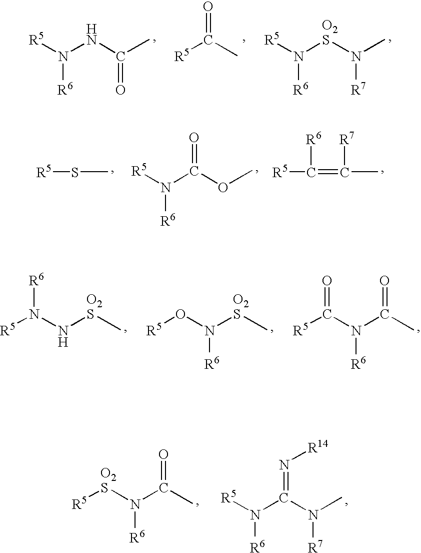
and the other symbols have the same meanings as defined above,
   or a pharmaceutically acceptable salt thereof.

46. A compound of the formula (I-2): 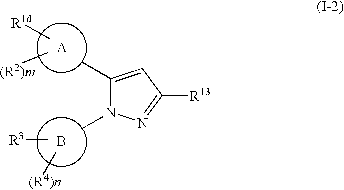
wherein R^{1d} is a group selected from the following formulae: 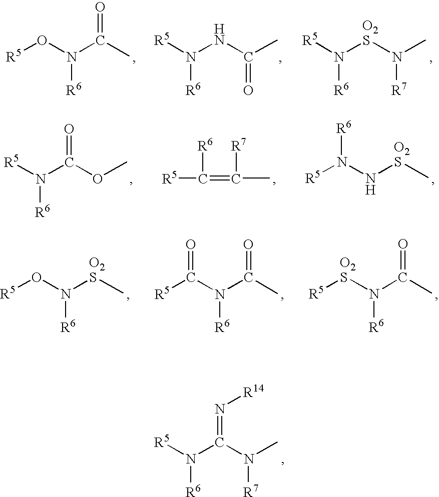
and the other symbols have the same meanings as defined above,
   or a pharmaceutically acceptable salt thereof.

47. A compound of the formula (I-3): 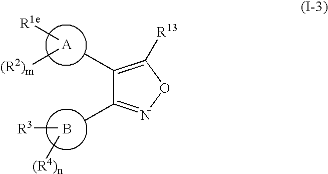
wherein R^{1e} is a group selected from the following formulae: 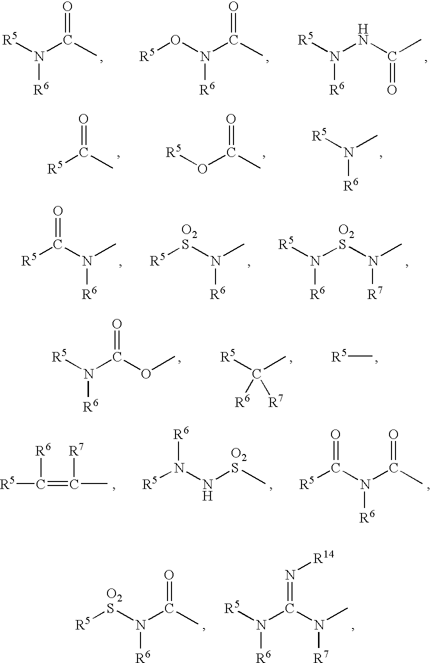
and the other symbols have the same meanings as defined above,
   or a pharmaceutically acceptable salt thereof.

48. A compound of the formula (I-4): 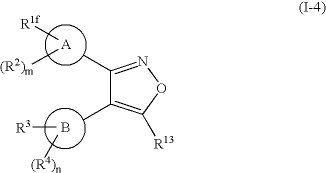
wherein R^{1f} is a group selected from the following formulae: 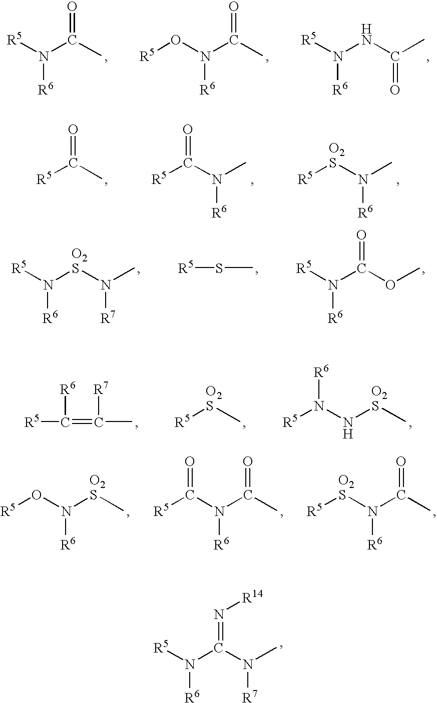
and the other symbols have the same meanings as defined above,
   or a pharmaceutically acceptable salt thereof.

49. The compound or a pharmaceutically acceptable salt thereof according to any one of the above 45 to 48, wherein the substituent(s) for the optionally substituted alkyl of R⁵, R⁶ or R⁷ are 1 to 7 independently selected halogen(s) and/or 1 to 3 groups selected from the following formulae: 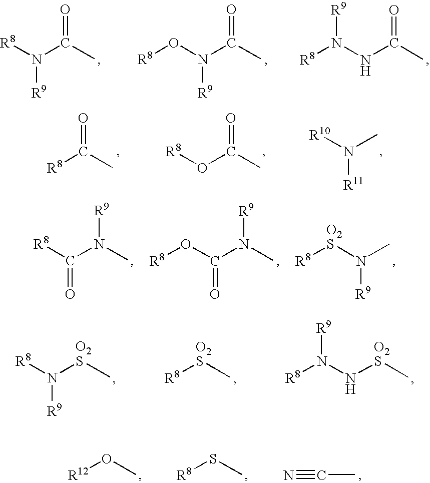
an optionally substituted heterocyclic group, and an optionally substituted aryl;
wherein each symbol has the same meaning as defined above.

50. A large conductance calcium-activated K channel opener, comprising a compound of the formula: 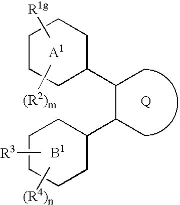
wherein R^{1g} is a group selected from the following formulae: 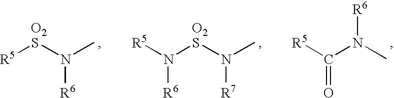
and the other symbols have the same meanings as defined above;
   or a pharmaceutically acceptable salt thereof, as an active ingredient.

51. The large conductance calcium-activated K channel opener according to the above 50, wherein R⁵ is a group selected from the following formulae: 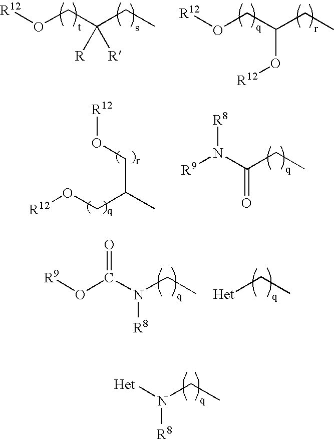
wherein each symbol has the same meaning as defined above.

52. A compound of the formula: 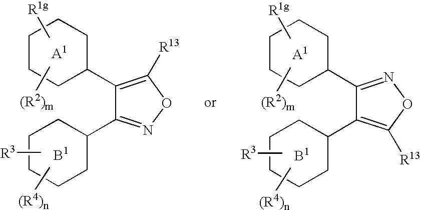
wherein R^{1g} is a group selected from the following formulae: 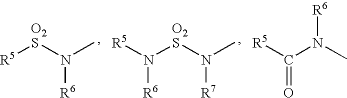
and the other symbols have the same meanings as defined above,
   or a pharmaceutically acceptable salt thereof.

53. The compound or a pharmaceutically acceptable salt thereof according to the above 52, wherein R⁵ is a group selected from the following formulae: 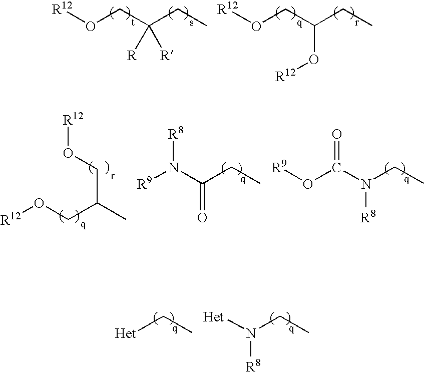
wherein each symbol has the same meaning as defined above.

54. A large conductance calcium-activated K channel opener, comprising a compound of the formula: 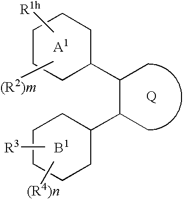
wherein R^{1h} is a group selected from the following formulae: 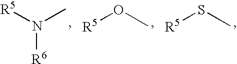
and the other symbols have the same meanings as defined above,
   or a pharmaceutically acceptable salt thereof, as an active ingredient.

55. The large conductance calcium-activated K channel opener according to the above 54, wherein R⁵ is a group selected from the following formulae: 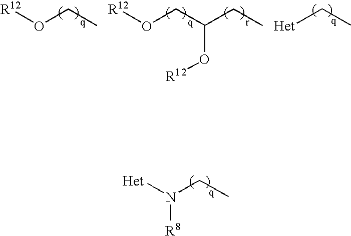
wherein each symbol has the same meaning as defined above.

56. A medicine comprising the compound or a pharmaceutically acceptable salt thereof according to any one of the above 15, 16, 19, 24 to 27, 29, 31, 32 to 34, 36 to 49, 52, and 53.

57. The medicine according to the above 56, which is a large conductance calcium-activated K channel opener.

58. The large conductance calcium-activated K channel opener according to the above 57, which is for the prophylaxis and/or treatment of pollakiuria, urinary incontinence, asthma or COPD.

Hereinafter, each group represented by the respective symbols in the present specification will be explained.

“Alkyl” and the alkyl in “alkoxyalkyl” and “alkylsulfonyl” is exemplified by a straight or branched C₁-C₆ alkyl, preferably by a straight or branched C₁-C₄ alkyl, and more specifically by methyl, ethyl, propyl, isopropyl, butyl, isobutyl, 1-methylpropyl, pentyl, hexyl, etc.

“Hydroxyalkyl” is exemplified by a straight or branched C₃-C₆ alkyl, preferably by a straight or branched C₁-C₄ alkyl which is substituted by hydroxyl(s), and more specifically by hydroxymethyl, 2-hydroxyethyl, 3-hydroxypropyl, 2-hydroxypropyl, 3-hydroxybutyl, 4-hydroxybutyl, etc.

“Alkoxy” and the alkoxy in “alkoxyalkyl” and “alkoxycarbonyl” is exemplified by a straight or branched C₁-C₆ alkoxy, preferably by a straight or branched C₁-C₄ alkoxy, and more specifically by methoxy, ethoxy, propoxy, isopropoxy, butoxy, isobutoxy, tert-butoxy, pentyloxy, hexyloxy, etc.

“Halogen” includes fluorine, chlorine, bromine, and iodine.

“Alkanoyl” is exemplified by a straight or branched C₁-C₆ alkanoyl, preferably by a straight or branched C₁-C₄ alkanoyl, more specifically by formyl, acetyl, propionyl, butyryl, pentanoyl, hexanoyl, etc.

“Haloalkyl” is exemplified by a C₁-C₆ alkyl, preferably a C₁-C₄ alkyl, which is substituted by halogen(s), and more specifically by chloromethyl, dichloromethyl, fluoromethyl, difluoromethyl, trifluoromethyl, 2,2,2-trifluoroethyl, 3-chloropropyl, 3-fluoropropyl, 4-chlorobutyl, 4-fluorobutyl, etc.

“Haloalkoxy” is exemplified by a C₁-C₆ alkoxy, preferably a C₁-C₄ alkoxy, which is substituted by halogen(s), and more specifically by chloromethoxy, dichloromethoxy, fluoromethoxy, difluoromethoxy, trifluoromethoxy, 2,2,2-trifluoroethoxy, perfluoroethoxy, 3-chloropropoxy, 3-fluoropropoxy, 4-chlorobutoxy, 4-fluorobutoxy, etc.

“Alkenyl” is exemplified by a straight or branched C₂-C₆ alkenyl, preferably by a straight or branched C₂-C₄ alkenyl, and more specifically by vinyl, allyl, 1-methyl-2-propenyl, 3-butenyl, 2-pentenyl, 3-hexenyl, etc.

“Aryl” is exemplified by a monocyclic, bicyclic or tricyclic C₆₋₁₄ aryl, preferably by a C₆₋₁₀ aryl, and more specifically by phenyl, naphthyl, phenanthryl, anthryl, etc. Phenyl and naphthyl are particularly preferred.

“Aralkyl” is exemplified by a straight or branched C₁-C₆ alkyl, preferably a straight or branched C₁-C₄ alkyl, which is substituted by aryl(s), and more specifically by benzyl, 2-phenylethyl, 1-phenylethyl, 3-phenylpropyl, etc. “Cycloalkyl” is exemplified by a C₃-C₈ cycloalkyl, preferably a C₃-C₆ cycloalkyl, and more specifically by cyclopropyl, cyclobutyl, cyclopentyl, and cyclohexyl, etc. “Cycloalkyl fused with an aryl” is exemplified by a C₃-C₈ cycloalkyl, preferably a C₃-C₆ cycloalkyl, which is fused with aryl (preferably phenyl). Specific examples thereof include indanyl, tetralinyl, etc. “Cycloalkyl” and “cycloalkyl fused with an aryl” may have substituent(s) which are exemplified by hydroxyl, a halogen, a C₁-C₄ alkyl, a C₁-C₄ alkoxy, etc., and preferably by hydroxyl. Specific example for the substituted cycloalkyl fused with an aryl includes 2-hydroxyindan-1-yl, etc.

“Heterocyclic group” is exemplified by a monocyclic or bicyclic 5 to 10-membered heterocyclic group, which may be partially or wholly saturated, containing 1 to 4 hetero atom(s) selected from nitrogen, oxygen and sulfur. The monocyclic or bicyclic heterocyclic group which may be partially or wholly saturated may be substituted by oxo.

The monocyclic heterocyclic group is preferably exemplified by a 5 to 7-membered heterocyclic group which may be partially or wholly saturated, containing 1 to 4 hetero atom(s) selected from nitrogen, oxygen and sulfur, and it is specifically exemplified by oxazolyl, pyrrolidinyl, pyrrolyl, pyrazolyl, pyridyl, pyrimidinyl, pyrazinyl, tetrazolyl, thiazolyl, piperidyl, piperazinyl, morpholinyl, tetrahydropyranyl, tetrahydrofuryl, imidazolidinyl, oxazolidinyl, etc.

The bicyclic heterocyclic group is preferably exemplified by a bicyclic heterocyclic group in which two of the same or different monocyclic heterocyclic groups above are fused, or a bicyclic heterocyclic group in which the above monocyclic heterocyclic group and benzene are fused, and it is specifically exemplified by dihydroindolyl, tetrahydroquinolyl, etc.

“Heterocyclic ring” of Ring A and Ring B is exemplified by a monocyclic or bicyclic 5 to 10-membered heterocyclic ring, which may be partially or wholly saturated, containing 1 to 4 hetero atom(s) selected from nitrogen, oxygen and sulfur. Specific examples thereof include thiophene, furan, pyrrole, pyrazole, thiazole, imidazole, oxazole, 1,2,4-oxadiazole, 1,3,4-oxadiazole, pyridine, pyrimidine, pyrazine, pyridazine, piperidine, piperazine, tetrahydropyridine, dihydropyridine, pyrrolidine, pyrroline, tetrahydroazepine, homopiperidine, morpholine, homopiperazine, tetrahydropyran, benzo[b]thiophene, benzo[b]furan, indole, 2,3-dihydroindole, 2,3-dihydrobenzo[b]furan, 1,4-benzodioxane, quinoline, 1,5-benzodioxepine, pyridooxazole, pyridoimidazole, benzoisoxazole, benzothiazole, pyridothiophene, and benzimidazole. Of these, pyridine, pyrazine, pyrimidine, pyridazine, thiazole, pyrazole, pyrrole, thiophene, quinoline and indole are preferable, and pyridine, thiophene and pyrazole are particularly preferable.

“Cycloalkane” of Ring B is exemplified by a C₃-C₈ cycloalkane, preferably a C₃-C₆ cycloalkane, and more specifically by cyclopropane, cyclobutane, cyclopentane, cyclohexane, etc. Cyclopropane is preferable.

“Cycloalkene” of Ring B is exemplified by a C₃-C₈ cycloalkene, preferably a C₃-C₆ cycloalkene, and more specifically by cyclopropene, cyclobutene, cyclopentene, cyclohexene, etc. Cyclohexene is preferable.

“Heterocyclic ring formed by R⁵ and R⁶ in combination with atom(s) to which they are bonded” and “heterocyclic ring formed by R⁸ and R⁹ in combination with atom(s) to which they are bonded” are exemplified by a saturated 5 to 8-membered monocyclic heterocycle which may have one or two hetero atom(s) (e.g. nitrogen, oxygen and sulfur, etc.). Specific examples thereof include pyrrolidine, piperidine, piperazine, morpholine, thiomorpholine, homopiperidine, etc.

The heterocyclic ring may be substituted, and the substituents are exemplified by (1) an alkyl which may be substituted by group(s) selected from (i) a halogen, (ii) hydroxyl, (iii) a haloalkoxy, (iv) an alkoxy which may be substituted by a halogen, an alkyl, phenyl, etc., (v) carbamoyl which may be substituted by alkyl(s), etc., (vi) cyano, (vii) an alkoxycarbonyl, (viii) carboxy, (ix) an amino which may be substituted by alkyl(s), phenyl(s), etc., and (x) an imino which may be substituted by an alkoxy, hydroxyl, etc.; (2) cyano; (3) a halogen; (4) an amino which may be substituted by alkyl(s), alkanoyl(s), cycloalkyl(s), etc.; (5) an alkenyl; (6) an imino which may be substituted by an alkoxy, hydroxyl, etc.; (7) a carbamoyl which may be substituted by alkyl(s), aralkyl(s), etc.; (8) an alkoxycarbonyl; (9) a heterocyclic group; etc.

Preferred examples of the substituent(s) for the substituted heterocyclic ring include an alkyl substituted by hydroxyl(s), and a 5- or 6-membered monocyclic heterocyclic group which may have 1 to 3 hetero atom(s) selected from nitrogen, oxygen and sulfur. Specifically hydroxymethyl and pyrimidyl are preferred.

Preferred examples of the substituent(s) for the substituted aryl of R⁵, R⁶ or R⁷ include an alkyl substituted by hydroxyl(s). Specific example of the substituted aryl is 2-hydroxymethylphenyl.

The substituent(s) for the substituted alkyl of R⁵, R⁶ and R⁷ is exemplified by 1 to 7 independently selected halogen(s) and/or by 1 to 3 groups selected from the following formulae: 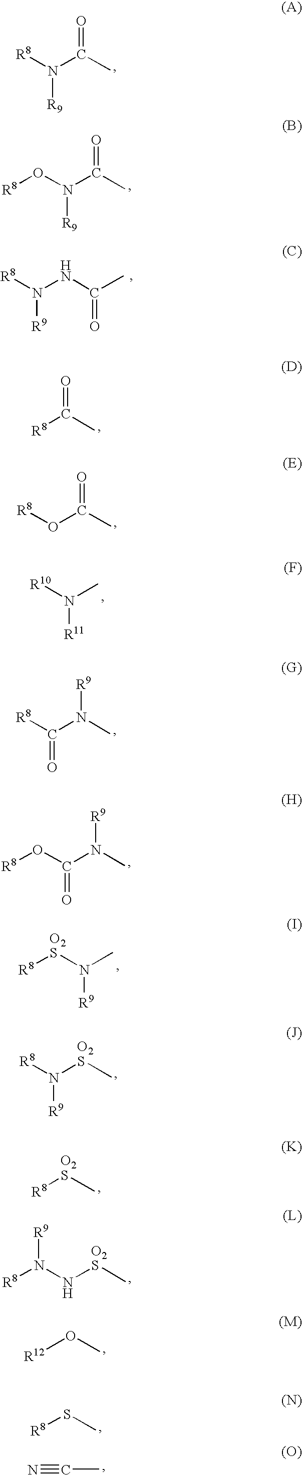
(P) an optionally substituted heterocyclic group, and (Q) an optionally substituted aryl
wherein each symbol has the same meaning as defined above.

Of the above, groups (A), (F), (H), (I), (M), (O), (P), and (Q) are preferred, and groups (A), (F), (H), (M), (P), and (Q) are particularly preferred.

The heterocyclic group as a substituent for the substituted alkyl of R⁵, R⁶, R⁷, or Het is preferably pyridyl, pyrazolyl, pyrazinyl, pyrimidinyl, tetrazolyl, or thiazolyl. The heterocyclic group may be substituted by an alkyl(s), haloalkyl(s), hydroxyl(s), alkoxy(s), etc., preferably by methyl(s), trifluoromethyl(s), hydroxyl(s), methoxy(s), etc.

The substituent of the substituted aryl of R⁸, R⁹, R¹⁰, R¹¹, and R¹² is exemplified by a halogen, hydroxyl, an alkoxy, an alkyl, a haloalkyl, etc.

The heterocyclic group of R⁸, R⁹, R¹⁰, R¹¹, and R¹² is preferably exemplified by pyridyl, pyrazolyl, pyrazinyl, pyrimidinyl, tetrazolyl, or tetrahydropyranyl. The heterocyclic group may be substituted by alkyl(s), haloalkyl(s), hydroxyl(s), alkoxy(s), etc. As the heterocyclic group of R¹⁰ or R¹¹, pyridyl is particularly preferred. As the heterocyclic group of R¹², pyrimidyl or tetrahydropyranyl is particularly preferred.

As the heterocyclic group of R¹³, particularly preferred is 4,5-dihydroxazole.

The substituent for the substituted carbamoyl and the substituted amino of R² or R⁴ is exemplified, respectively, by an alkyl which may be substituted by halogen(s), hydroxyl(s), alkoxy(s), amino(s), or mono- or di-alkylamino(s), etc.

The substituent for the substituted alkyl of R² or R⁴ is exemplified by hydroxyl, an alkoxy, a halogen, etc. Examples of the substituted alkyl include hydroxymethyl, 2-hydroxyethyl, methoxymethyl, trifluoromethyl, etc.

The substituent for the substituted alkyl of R¹³ is exemplified by (1) a halogen, (2) hydroxyl, (3) a haloalkoxy, (4) an alkoxy which may be substituted by halogen(s), alkoxy(s), phenyl(s), etc., (5) a carbamoyl which may be substituted by alkyl(s), hydroxyl(s), etc., (6) cyano, (7) an alkoxycarbonyl, (8) carboxy, (9) an amino which may be substituted by alkyl(s), phenyl(s), etc., and (10) an imino which may be substituted by an alkoxy, hydroxyl, etc. Preferred is (1) a halogen, (2) hydroxyl, (4) an alkoxy which may be substituted by halogen(s), alkoxy(s), phenyl(s), etc., (6) cyano, (8) carboxy, (9) an amino which may be substituted by alkyl(s), phenyl(s), etc., and (10) an imino which may be substituted by an alkoxy, hydroxyl, etc.

The substituent for the substituted amino of R¹³ may be an alkyl, phenyl, etc.

The substituent for the substituted carbamoyl of R¹³ may be an alkyl, etc.

The substituent for the substituted alkyl of R¹⁴ may be cyano, a halogen, hydroxyl, an alkoxy, etc.

The substituent for the substituted amino of Z may be an alkyl, etc.

Examples of the pharmaceutically acceptable salts of the compound of the present invention may include, for example, inorganic acid salts such as hydrochloride, sulfate, phosphate or hydrobromide, and organic acid salts such as acetate, fumarate, oxalate, citrate, methanesulfonate, benzenesulfonate, tosylate or maleate. In addition, in case of compound having an acidic group such as carboxy, salts with a base (for example, alkali metal salts such as a sodium salt and a potassium salt, alkaline earth metal salts such as a calcium salt, organic base salts such as a triethylamine salt, or amino acid salts such as a lysine salt) can be mentioned.

The compound of the present invention or the pharmaceutically acceptable salt thereof includes any of its internal salts, and solvates such as hydrates.

In the compound (I) of the present invention, an optical isomer based on an asymmetric carbon may be present, and any of the isomers and a mixture thereof may be encompassed in the compound (I) of the present invention. In addition, cis form and trans form may be present, in case that the compound (I) of the present invention has a double bond or a cycloalkanediyl moiety, and a tautomer may be present based on an unsaturated bond such as carbonyl in the compound (I) of the present invention, and any of these isomers and a mixture thereof may be encompassed in the compound (I) of the present invention.

The compound (I) of the present invention may be prepared by the following methods.

Further, unless otherwise specified, the following methods will be explained using: 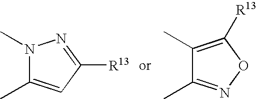
as a pyrazole or isoxazole of Ring Q. If other corresponding starting material is used, however, the compound having the following moiety: 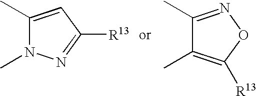
may also be prepared in a similar manner.

The reaction with respect to R¹ may be performed in a manner similar to the reaction with respect to R³

Method 1:

The compound in which Ring Q is pyrazole and R¹³ is an optionally substituted alkyl, an alkenyl or a heterocyclic group may be prepared by the following method: 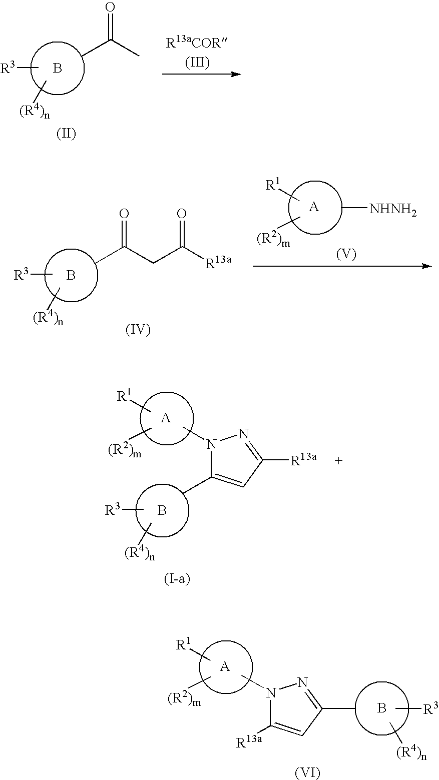
wherein R^{13a} is an optionally substituted alkyl, an alkenyl or a heterocyclic group, R″ is an alkoxy such as methoxy and ethoxy or imidazole, and the other symbols have the same meanings as defined above.

The reaction between Compounds (II) and (III) may be carried out in the presence of a base such as sodium methoxide, sodium ethoxide, and sodium hydride, according to the method of J. Am. Chem. Soc., Vol. 72, pp. 2948-2952, 1950.

Compound (IV) is reacted with Compound (V) or a salt thereof (e.g. a hydrochloride) in a solvent (e.g. methanol, ethanol, isopropyl alcohol, ethylene glycol, DMF, DMSO, acetic acid, water, or a mixture thereof) at room temperature to the refluxing temperature of the solvent for 1 to 24 hours to give a mixture of Compounds (I-a) and (VI). The resulting reaction mixture is subjected to recrystallization or chromatography so that Compound (I-a) can be isolated.

Method 2:

Compound (I-a) may also be prepared by the following method: 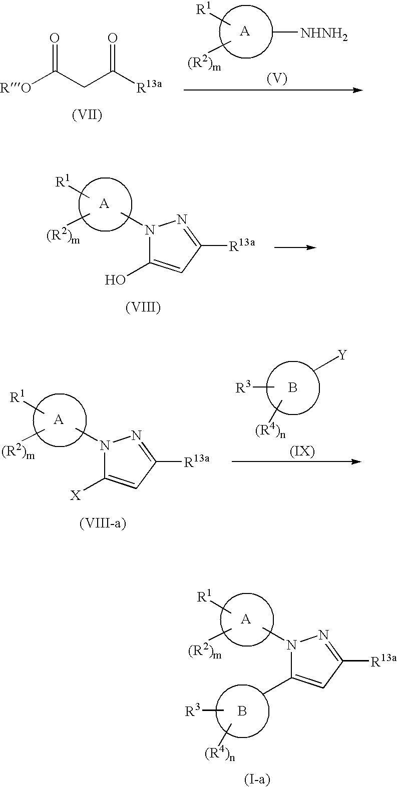
wherein R′″ is a C₁-C₄ alkyl such as methyl and ethyl; X is a leaving group such as a halogen or an optionally substituted alkylsulfonyloxy (preferably trifluoromethanesulfonyloxy); Y is —B(OH)₂, —B(OR^{a})₂ or —Sn(R^{a})₃ wherein R^{a} is an alkyl; and the other symbols have the same meanings as defined above.

The reaction between Compounds (VII) and (V) may be carried out in a manner similar to the reaction between Compounds (IV) and (V) in Method 1.

Compound (VIII) is converted into Compound (VIII-a) by a conventional method using a halogenating agent (e.g. phosphorus oxychloride and phosphorus oxybromide) or a sulfonylating agent (e.g. trifluoromethanesulfonic anhydride), and then Compound (VIII-a) is reacted with Compound (IX) in the presence of a palladium catalyst to give Compound (I-a). As the palladium catalyst, there may be suitably used, for example, a zero-valent or divalent palladium catalyst such as tetrakis(triphenylphosphine)-palladium(0), bis(triphenylphosphine)palladium(II) chloride and palladium(II) acetate. In case of using Compound (IX) in which Y is —B(OH)₂ or —B(OR)₂, it is preferable to add a base in the reaction. As the base, there may used an inorganic base such as alkali metal carbonate, alkali metal hydroxide, alkali metal phosphate, and alkali metal fluoride, or an organic base such as triethylamine. Any solvent may be used as long as it has no adverse effect on the reactions. Examples of such solvent include DME, THF, dioxane, DMF, dimethylacetamide, toluene, benzene, and a mixture thereof. The present reaction generally proceeds at 60 to 150° C., suitably at 80 to 120° C., for generally from 1 to 24 hours.

Method 3:

The compound in which Ring Q is pyrazole and R¹³ is amino or a halogen may be prepared by the following method: 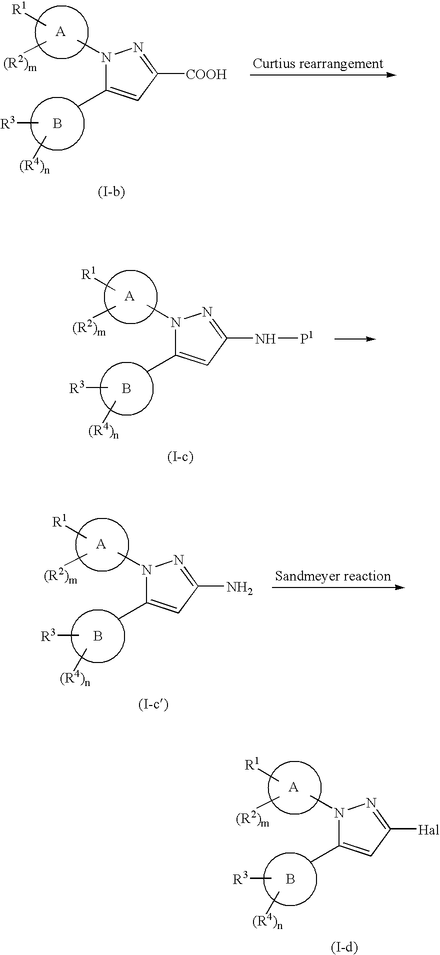
wherein P¹ is tert-butoxycarbonyl or benzyloxycarbonyl, and each symbol has the same meaning as defined above.

Compound (I-b) is reacted with an azidating agent (e.g. diphenylphosphoryl azide) in a solvent (e.g. THF, diethyl ether, ethylene glycol dimethyl ether, DMF, DMSO and dioxane) in the presence of an alcohol (e.g. tert-butanol and benzyl alcohol) and a base (e.g. triethylamine and diisopropylethylamine), at −20° C. to 150° C. for 30 minutes to 10 hours to give Compound (I-c). In this process, the azidation reaction may also be performed using an activating agent (e.g. methyl chlorocarbonate, ethyl chlorocarbonate, isopropyl chlorocarbonate, isobutyl chlorocarbonate, and phenyl chlorocarbonate) and sodium azide.

Compound (I-c) is treated with an acid (e.g. hydrochloric acid and trifluoroacetic acid), or subjected to catalytic hydrogenation, according to a conventional method, so that Compound (I-c′) can be prepared.

Compound (I-c′) is converted into a diazo compound using sodium nitrite, nitrous acid, organic nitrite (e.g. isopentyl nitrite), etc, in a solvent (e.g. water, acetic acid, hydrochloric acid, hydrobromic acid, nitric acid, dilute sulfuric acid, or a mixture thereof), and then the diazo compound is reacted with a nucleophilic reagent (e.g. fluoroboric acid, hydrochloric acid-cuprous chloride, hydrobromic acid-cuprous bromide, iodine, potassium iodide, and sodium iodide) to give Compound (I-d). The reaction generally proceeds at −20° C. to 100° C., and generally for 10 minutes to 10 hours.

Method 4:

The compound in which Ring Q is pyrazole and R¹³ is carbamoyl, cyano or methyl substituted by an optionally substituted imino may be prepared according to the method described in J. Med. Chem., Vol. 40, pp. 1347-1365, 1997 and JP09-506350.

Method 5:

Compound (I-e) in which ring Q is isoxazole and R¹³ is an optionally substituted alkyl, an alkenyl or a heterocyclic group may be prepared by the following method: 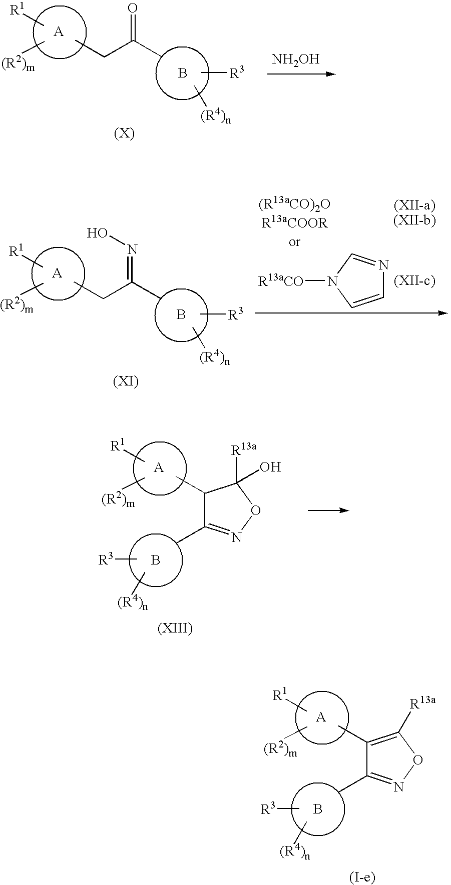
wherein each symbol has the same meaning as defined above.

Compound (XI) is prepared by the reaction of Compound (X) with hydroxylamine or a salt thereof (e.g. a hydrochloride) in a solvent (e.g. water, methanol, ethanol, or a mixture thereof). The reaction generally proceeds at 0° C. to the refluxing temperature of the solvent, preferably at room temperature to 50° C., and generally for 1 to 24 hours. In case of using a salt of hydroxylamine, the reaction is preferably carried out in the presence of an alkali (e.g. sodium bicarbonate).

Compound (XI) is reacted with Compound (XII-a), (XII-b) or (XII-c) in a solvent (e.g. THF and diethyl ether), in the presence of a base (e.g. n-butyl lithium and lithium diisopropylamide) to give Compound (XIII). The reaction proceeds generally at −78° C. to ice-cooling temperature, and generally for 1 to 24 hours.

Compound (XIII) is treated with an acid (e.g. hydrochloric acid, sulfuric acid and p-toluenesulfonic acid) in a solvent (e.g. methanol, ethanol, benzene, toluene, xylene, and chloroform) to give Compound (I-e). The reaction generally proceeds at 0° C. to the refluxing temperature of the solvent, and generally for 1 to 24 hours.

Method 6:

Compound (I-f) may also be prepared by the following method: 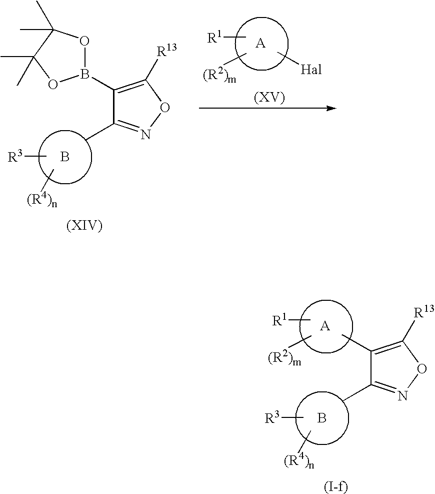
wherein each symbol has the same meaning as defined above.

The reaction between Compound (XIV) which may be prepared according to the method described in Chem. Commun., 1558-59, 2001, and Compounds (XV) may be carried out in a manner similar to the reaction between Compound (VIII-a) and Compound (IX) in Method 2 to give Compound (I-f).

Method 7:

Compound (I) may be prepared by the following method: 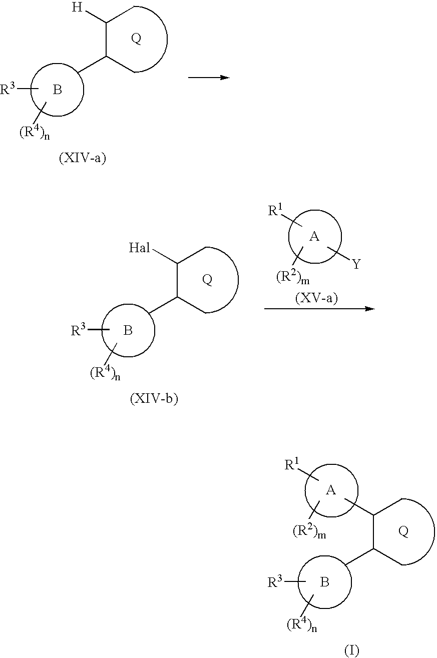
wherein each symbol has the same meaning as defined above.

Compound (XIV-a) is halogenated with a halogenating agent (e.g. bromine, chlorine, iodine, and N-bromosuccinimide) by a conventional method to give Compound (XIV-b). The reaction between Compound (XIV-b) and Compound (XV-a) may be carried out in a manner similar to the reaction between Compound (VIII-a) and Compound (IX).

Method 8:

The compound in which R¹ is —SO₂N(R⁵)(R⁶) may be prepared by the following method: 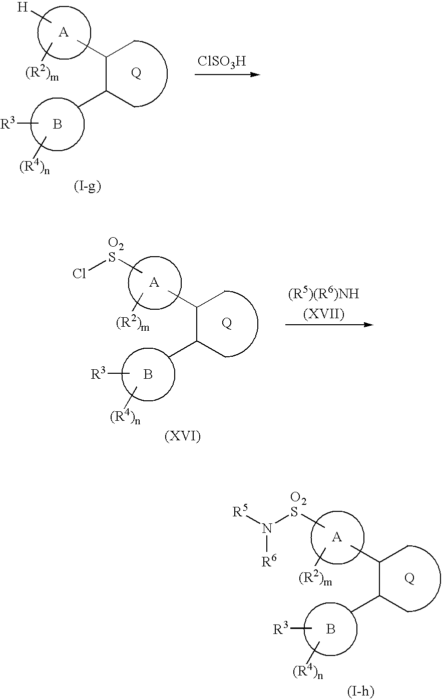
wherein each symbol has the same meaning as defined above.

Compound (I-g) is treated with chlorosulfonic acid in a solvent (e.g. chloroform and methylene chloride), at ice-cooling temperature to the refluxing temperature of the solvent, preferably at room temperature, for 1 to 48 hours to give Compound (XVI).

Compound (XVI) is reacted with Compound (XVII) in the presence of a base (e.g. triethylamine) if necessary or using an excess amount of Compound (XVII) at ice-cooling temperature to room temperature for 1 to 24 hours to give Compound (I-h).

Method 9:

The compound in which R¹ is —NH₂ may be prepared by Method 6 or 7 or by the following method: 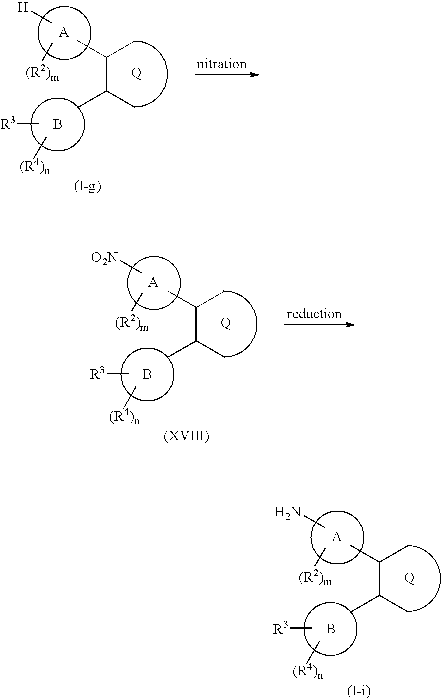
wherein each symbol has the same meaning as defined above.

Compound (I-g) is treated with nitric acid, mixed acid, acetyl nitrate, etc., in the presence or in the absence of a solvent (e.g. acetic acid, acetic anhydride, c. sulfuric acid, chloroform, dichloromethane, carbon disulfide, dichloroethane, or a mixture thereof) to give Compound (XVIII). The reaction generally proceeds at −20° C. to 100° C., and generally for 30 minutes to 12 hours.

Compound (XVIII) is reduced in a solvent (e.g. water, methanol, ethanol, tert-butyl alcohol, THF, dioxane, ethyl acetate, acetic acid, xylene, DMF, DMSO, or a mixture thereof) to give Compound (I-i). The reduction reaction may be carried out using a reducing agent such as sodium borohydride, lithium borohydride and lithium aluminum hydride or using a metal (e.g. iron, zinc and tin) or may be carried out by catalytic hydrogenation with a transition metal (e.g. palladium-carbon, platinum oxide, Raney nickel, rhodium, and ruthenium). In case of carrying out the catalytic hydrogenation, the hydrogen source may be formic acid, ammonium formate, 1,4-cyclohexadiene, or the like. The reaction proceeds generally at −20° C. to 150° C., and generally for 30 minutes to 48 hours.

Method 10:

The compound in which R¹ is —NHCOR⁵ or —NHSO₂R⁵ may be prepared by Method 6 or 7 or by the following method: 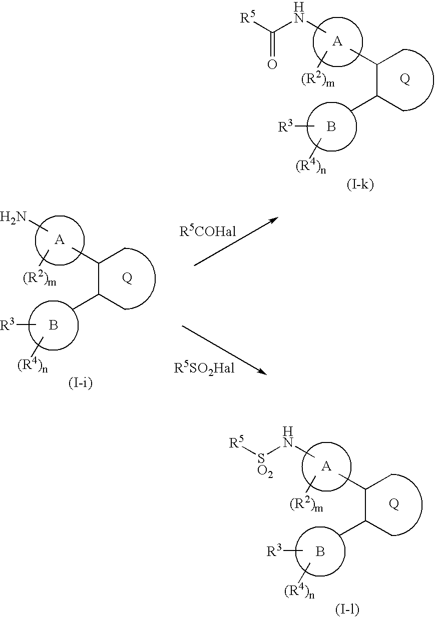
wherein each symbol has the same meaning as defined above.

N-acylation or N-sulfonylation of Compound (I-i) may be carried out in a solvent, in the presence of a base. Examples of the solvent include THF, dioxane, diethyl ether, ethylene glycol dimethyl ether, benzene, dichloromethane, dichloroethane, chloroform, toluene, xylene, DMF, DMSO, water, and a mixture thereof. Examples of the base include potassium carbonate, potassium bicarbonate, sodium carbonate, sodium bicarbonate, sodium hydroxide, potassium hydroxide, triethylamine, diisopropylethylamine, 1,8-diazabicyclo[5.4.0]undec-7-ene(DBU), pyridine, and 4-dimethylaminopyridine. The reaction proceeds generally at −80° C. to 150° C., and generally for 30 minutes to 48 hours.

Method 11:

The compound in which R¹ is —COOR⁵ or —CONR⁵R⁶ may be prepared by the following method: 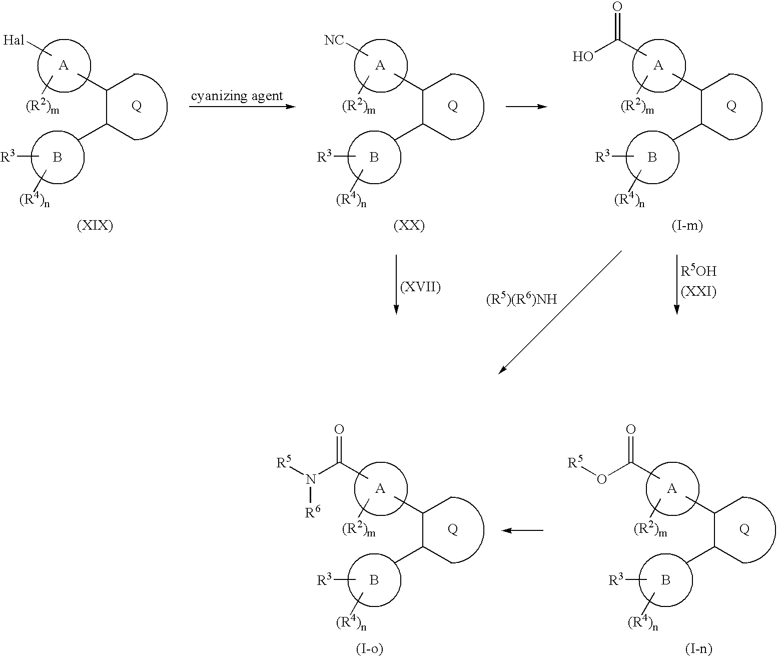
wherein each symbol has the same meaning as defined above.

Compound (XIX) is reacted with a cyanizing agent (e.g. sodium cyanide and cuprous cyanide) in a solvent (e.g. acetonitrile, DMSO, DMF, or a mixture thereof), at room temperature to 100° C. for 1 to 24 hours to give Compound (XX). Compound (XX) may also be prepared using a palladium catalyst such as tetrakis(triphenylphosphine)-palladium and a cyanizing agent such as zinc cyanide and potassium cyanide.

Compound (XX) is hydrolyzed with an acid (e.g. hydrochloric acid and sulfuric acid) or an alkali (e.g. sodium hydroxide and potassium hydroxide) in a solvent (e.g. water, methanol, ethanol, isopropyl alcohol, tert-butyl alcohol, ethylene glycol, diethylene glycol, or a mixture thereof) to give Compound (I-m). The reaction proceeds generally at −20° C. to 150° C., and generally for 30 minutes to 48 hours. Alternatively, Compound (I-m) may also be prepared by Method 6 or 7.

Compound (I-o) or Compound (I-n) may be prepared by any of the following methods:

(1) Compound (I-m) is converted into an acid halide by treating it with a halogenating agent (e.g. thionyl chloride), and the acid halide is reacted with Compound (XVII) or Compound (XXI) in the presence of a base (e.g. sodium bicarbonate, potassium carbonate, triethylamine, and pyridine) at −20° C. to room temperature for 30 minutes to 24 hours to give Compound (I-o) or Compound (I-n). Compound (XX) may be hydrolyzed with an alkali (e.g. sodium hydroxide and potassium hydroxide) in a solvent (e.g. water, methanol, ethanol, isopropyl alcohol, tertbutyl alcohol, ethylene glycol, diethylene glycol, or a mixture thereof) to give Compound (I-o) in which R⁵ and R⁶ are hydrogen.

(2) Compound (I-m) is condensed with Compound (XVII) or Compound (XXI) in a solvent (e.g. DMF, THF and dioxane) if necessary, in the presence of a condensation agent (e.g. 1,3-dicyclohexylcarbodiimide, 1-ethyl-3-(3-dimethylaminopropyl)carbodiimide, carbonyldiimidazole, and diethyl cyanophosphate) to give Compound (I-o) or Compound (I-n). The reaction proceeds generally at 0° C. to 100° C., and generally for 30 minutes to 24 hours. The reaction using the condensation agent may also be carried out in the presence of 1-hydroxybenzotriazole, N-hydroxysuccinimide or the like, if necessary.

(3) Compound (I-m) is converted into a carbonate (a mixed acid anhydride with methyl chlorocarbonate, ethyl chlorocarbonate etc.). The carbonate is then condensed with Compound (XVII) or Compound (XXI) in the presence of a base (e.g. triethylamine and pyridine) in a suitable solvent (e.g. THF, toluene, nitrobenzene, or a mixed solvent thereof) at room temperature to the refluxing temperature of the solvent for 1 to 24 hours to give Compound (I-o) or Compound (I-n).

Method 12:

The compound in which R¹ is —O—R⁵ or —S—R⁵ may be prepared by the following method: 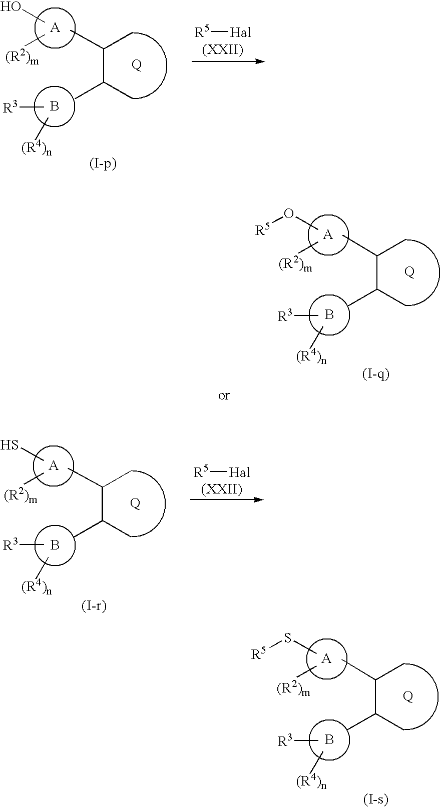
wherein each symbol has the same meaning as defined above.

Compound (I-p) or Compound (I-r) is reacted with Compound (XXII) in a suitable solvent (e.g. water, DMSO, DMF, toluene, THF, or a mixed solvent thereof), in the presence of a base (e.g. sodium hydroxide and sodium hydride) at −20° C. to the refluxing temperature of the solvent for 1 to 24 hours to give Compound (I-q) or Compound (I-s).

Method 13:

The compound in which R¹ is —SO₂—R⁵ may be prepared by Method 6 or 7, or by the following method: 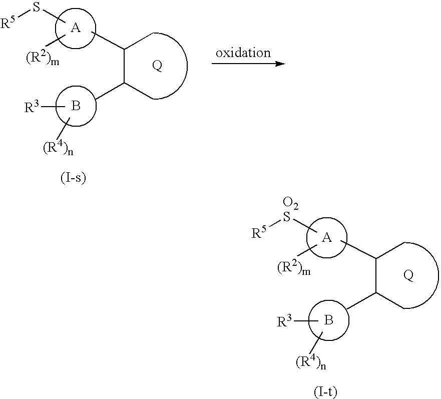
wherein each symbol has the same meaning as defined above.

Compound (I-s) is reacted with an oxidizing agent (e.g. meta-chloroperbenzoic acid and hydrogen peroxide) in a suitable solvent (e.g. acetic acid, dioxane, chloroform, methylene chloride, or a mixture thereof) at 0° C. to 100° C. for 30 minutes to 24 hours to give Compound (I-t).

Method 14:

The compound in which R¹ is —SO₂N(R⁶)OR⁵ or —CON(R⁶)OR⁵ or the compound in which R¹ is —SO₂NHN(R⁵)(R⁶) or —CONHN(R⁵)(R⁶) may be prepared by the following method: 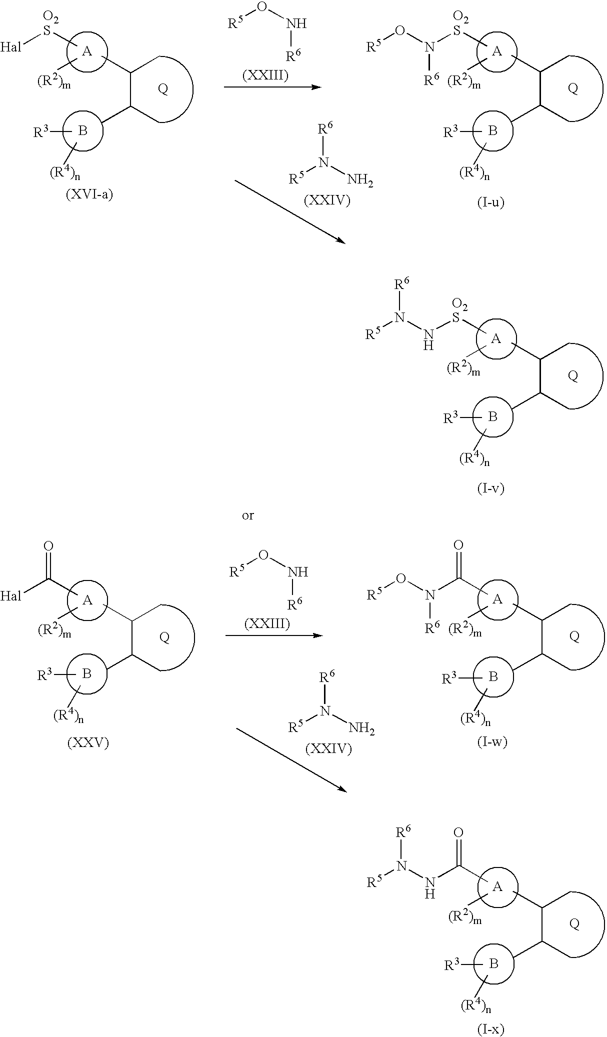
wherein Hal is a halogen such as chlorine and bromine, and the other symbols have the same meanings as defined above.

Compound (XVI-a) or Compound (XXV) is reacted with Compound (XXIII) in a suitable solvent (e.g. water, ethyl acetate, DMF, DMSO, chloroform, methylene chloride, THF, or a mixture thereof), in the presence of a base (e.g. triethylamine, sodium bicarbonate and potassium carbonate) at a temperature of from ice-cooling temperature to the refluxing temperature of the solvent for 1 to 24 hours to give Compound (I-u) or Compound (I-w).

Compound (XVI-a) or Compound (XXV) is reacted with Compound (XXIV) in a manner similar to the above to give Compound (I-v) or Compound (I-x).

Method 15:

The compound in which R¹ is —COR⁵ may be prepared by the following method: 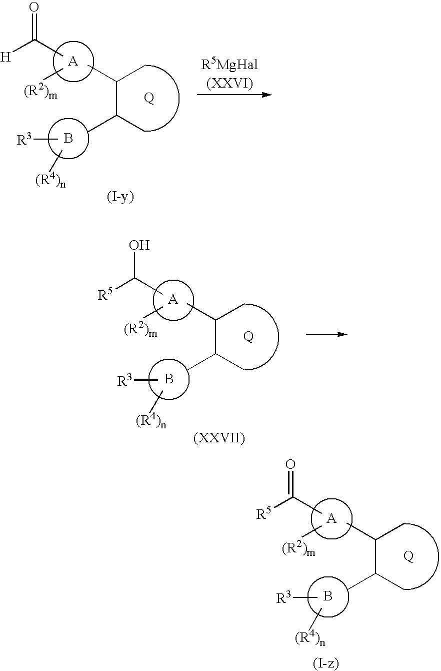
wherein each symbol has the same meaning as defined above.

Compound (I-y) is subjected to Grignard reaction with Compound (XXVI) in a solvent (e.g. THF, diethyl ether, ethylene glycol dimethyl-ether, benzene, toluene, xylene, and dioxane) at −20 to 100° C. for 30 minutes to 24 hours to give Compound (XXVII).

Compound (XXVII) is reacted with an oxidizing agent [e.g. chromic acid-sulfuric acid, chromium(VI) oxide-sulfuric acid-acetone (Jones reagent), chromium(VI) oxide-pyridine complex (Collins reagent), dichromate (e.g. sodium dichromate and potassium dichromate)-sulfuric acid, pyridinium chlorochromate (PCC), manganese dioxide, DMSO-electrophilic activating reagent (e.g. dicyclohexylcarbodiimide, acetic anhydride, phosphorus pentaoxide, a sulfur trioxide-pyridine complex, trifluoroacetic anhydride, oxalyl chloride, and halogen), sodium hypochlorite, potassium hypochlorite, and sodium bromite] at −20° C. to 100° C. for 30 minutes to 24 hours to give Compound (I-z).

Method 16:

The compound in which R¹ is —NHSO₂N(R⁵)(R⁶) may be prepared by the following method: 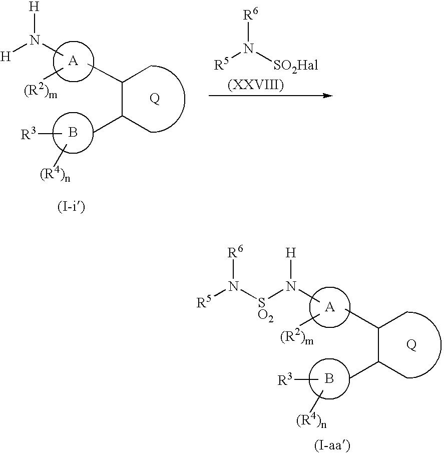
wherein each symbol has the same meaning as defined above.

Compound (I-i′) is reacted with Compound (XXVIII) in a manner similar to Method 11 to give Compound (I-aa′).

Method 17:

The compound in which R¹ is —OCON(R⁵)(R⁶) may be prepared by the following method: 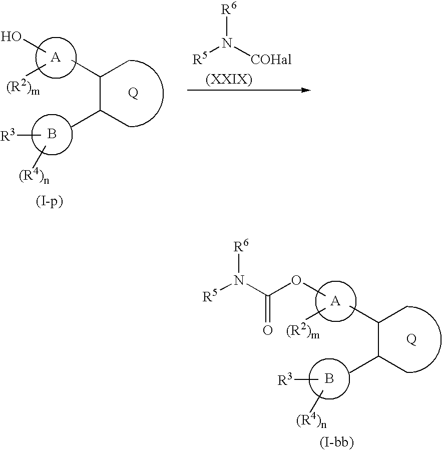
wherein each symbol has the same meaning as defined above.

Compound (I-p) is reacted with Compound (XXIX) in a manner similar to Method 11 to give Compound (I-bb).

Method 18:

The compound in which R¹ is —C(R⁷)═C(R⁵)(R⁶) may be prepared by the following method: 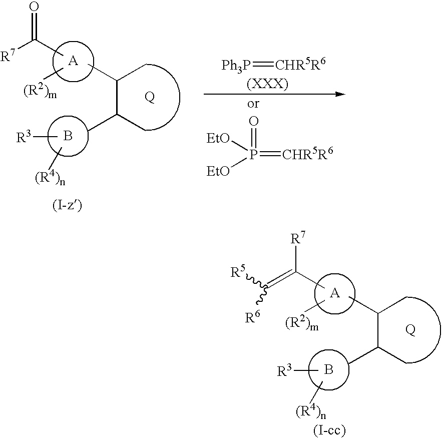
wherein Ph is phenyl, and the other symbols have the same meanings as defined above.

Compound (I-z′) is subjected to Wittig reaction with Compound (XXX) at −20° C. to 150° C. for 30 minutes to 24 hours to give Compound (I-cc). Examples of the solvent for use in this reaction include water, methanol, ethanol, tert-butyl alcohol, THF, diethyl ether, ethylene glycol dimethyl ether, DMF, DMSO, benzene, toluene, xylene, dioxane, methylene chloride, chloroform, dichloroethane, and acetonitrile. Examples of the base for use in this reaction include sodium methoxide, sodium ethoxide, potassium tert-butoxide, sodium hydride, potassium hydride, lithium diisopropylamide, butyl lithium, lithium hexamethyldisilazane, triethylamine, diisopropylethylamine, pyridine, and DBU.

Method 19:

Compound (I-dd) in which Ring Q is isoxazole and R¹³ is an optionally substituted alkyl may be prepared by the following method: 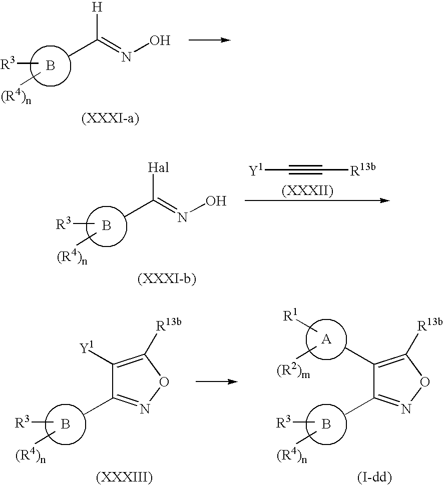
wherein Y¹ is —B(OR^{a})₂ or —Sn(R^{a})₃ wherein R^{a} is an alkyl, R^{13b} is an optionally substituted alkyl, and the other symbols have the same meanings as defined above.

Compound (XXXI-a) is halogenated by a conventional method using a halogenating agent (e.g. chlorine, N-chlorosuccinimide, and sodium hypochlorite) to give Compound (XXXI-b).

Compound (XXXI-b) is reacted with Compound (XXXII) in a solvent (e.g. diethyl ether, diisopropyl ether, THF, dioxane, acetone, methyl ethyl ketone, methylene chloride, 1,2-dichloroethane, carbon tetrachloride, benzene, toluene, xylene, DMF, DMSO, methanol, ethanol, propanol, isopropanol, butanol, ethyl acetate, water, or a mixture thereof), in the presence of a base (e.g. sodium bicarbonate, potassium bicarbonate, sodium carbonate, potassium carbonate, calcium carbonate, pyridine, and triethylamine) to give Compound (XXXIII). The reaction proceeds generally at −20° C. to 150° C., preferably at 0° C. to 100° C., and generally for 1 to 24 hours.

Alternatively, Compound (XXXIII) can also be prepared according to the method described in Acta Chemica Scandinavica, Vol. 48, pp. 61-67, 1994, by reacting Compound (XXXI-a) with a halogenating agent and Compound (XXXII), without isolating Compound (XXXI-b).

The resulting Compound (XXXIII) is reacted in a manner similar to the reaction between Compound (VIII-a) and Compound (IX) in Method 2 to give Compound (I-dd).

Method 20:

Compound (I-ee) in which ring Q is isoxazole, R¹ is CON(R⁵)(R⁶), R² is hydrogen, and Ring A is pyrroline, tetrahydropyridine or tetrahydroazepine may be prepared by the following method: 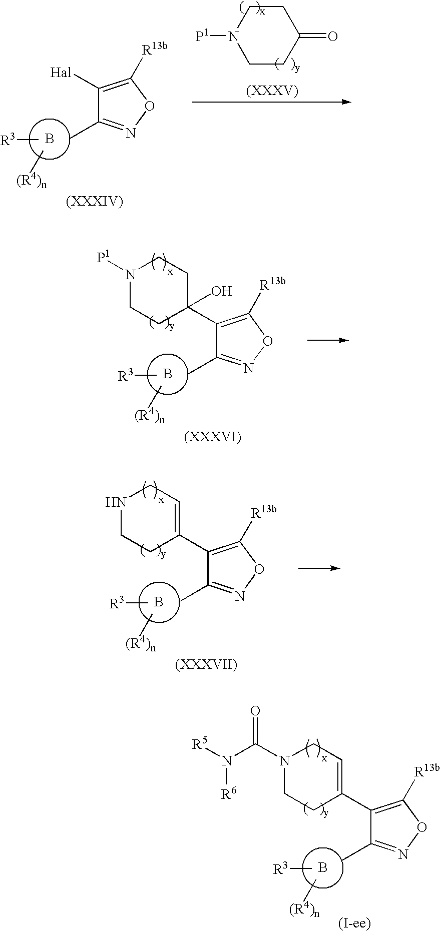
wherein P¹ is an amino-protecting group such as Boc, x is 0 or 1, y is 1 or 2, and the other symbols have the same meanings as defined above.

Compound (XXXIV) is converted into a lithio compound by treatment with a base (e.g. butyl lithium and lithium diisopropylamide) in a suitable solvent (e.g. THF, dioxane, dimethyl ether, and DME), at −78° C. to room temperature, which is then reacted with Compound (XXXV) for 1 to 24 hours to give Compound (XXXVI).

Compound (XXXVI) is reacted with an acid [such as trimethylsilyl polyphosphate (PPSE)], or Compound (XXXVI) is converted into a halide or a sulfonate ester, which is treated with a base (e.g. pyridine and DBU) and subjected to de-protection to give Compound (XXXVII). This reaction may be carried out in a suitable solvent (e.g. methylene chloride, chloroform, THF, dioxane, DMF, and DMSO) at 0° C. to the refluxing temperature of the solvent for 1 to 24 hours.

The resulting Compound (XXXVII) is reacted with triphosgene and HN(R⁵)(R⁶) in a suitable solvent (e.g. methylene chloride, chloroform, THF, dioxane, DMF, and DMSO), at ice-cooling temperature to room temperature for 1 to 24 hours to give Compound (I-ee). This reaction may also be carried out using (R⁵)(R⁶)NCOHal or (R⁵)(R⁶)NCO and a base (e.g. pyridine and triethylamine) in place of triphosgene and HN(R⁵)(R⁶).

Alternatively, the hydroxyl group of the resulting Compound (XXXVI) may be converted into OC(═S)SMe, and then the resulting compound is treated with tributyltin hydride and a radical initiator (e.g. 2,2′-azobisisobutyronitrile (AIBN)), to give the compound in which Ring A is pyrrolidine, piperidine or homopiperidine.

Method 21:

The compound in which R¹ is —CON(R⁶) COR⁵ or —CON(R⁶) SO₂R⁵ may be prepared by the following method: 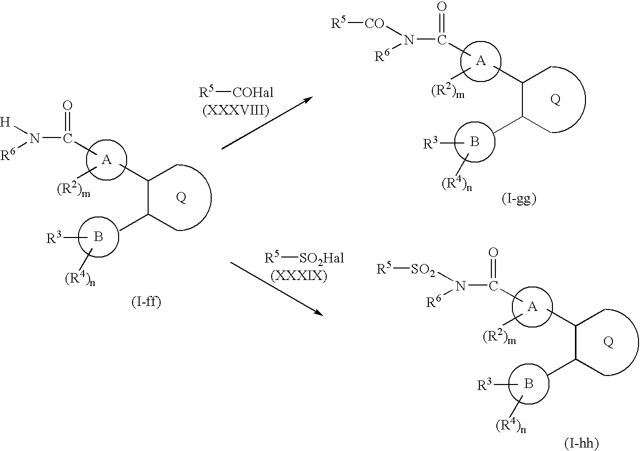
wherein each symbol has the same meaning as defined above.

Compound (I-ff) is reacted with Compound (XXXVIII) or Compound (XXXIX) in the presence of a base (e.g. sodium bicarbonate, potassium carbonate, triethylamine, and pyridine) at −20° C. to room temperature for 30 minutes to 24 hours to give Compound (I-gg) or (I-hh).

Method 22:

A compound in which Ring Q is isoxazole and R¹³ is an alkyl substituted by halogen(s) can be prepared by the following method. 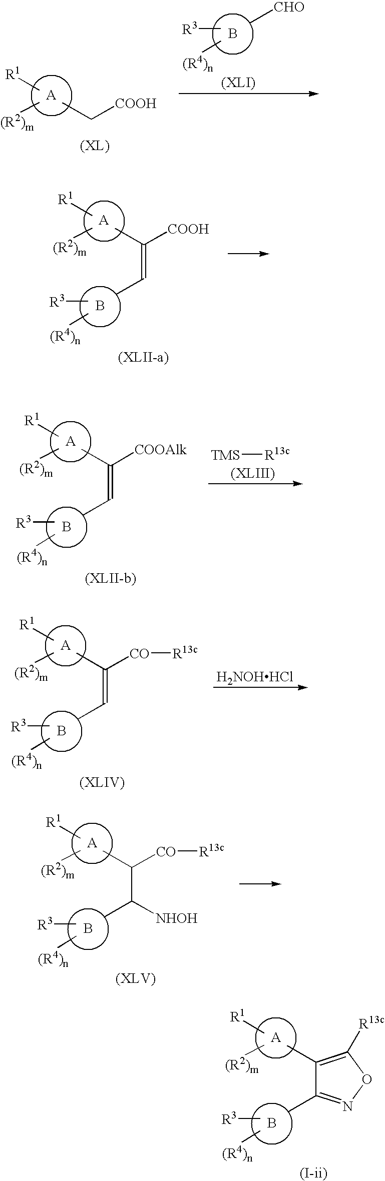
wherein R¹³c is an alkyl substituted by halogen(s), Alk is an alkyl, and the other symbols have the same meaning as defined above.

The present reaction can be carried out in accordance with the method described in Drug Development Research 51, 273-286 (2000).

Compound (XL) is reacted with Compound (XLI) in a suitable solvent (e.g. benzene, toluene, xylene, acetic anhydride) in the presence of a base (e.g. triethylamine, diisopropylethylamine and pyridine) at the refluxing temperature of the solvent for 1 to 48 hours to give Compound (XLII-a)

Compound (XLII-a) is esterified in accordance with Method 11 using an alcohol (e.g. methanol), and Compound (XLII-b) is reacted with Compound (XLIII) in a suitable solvent (e.g. DME and THF) in the presence of a catalyst (e.g. cesium fluoride) at 0° C. to 100° C. for 1 to 24 hours. Then, a suitable acid (e.g. hydrochloric acid and sulfuric acid) is added thereto so that a reaction proceeds for 1 to 24 hours to give Compound (XLIV).

Compound (XLIV) is reacted with hydroxylamine hydrochloride in a suitable solvent (e.g. methanol, ethanol, isopropanol) in the presence of a base (e.g. sodium acetate, triethylamine, sodium carbonate and sodium bicarbonate) at the refluxing temperature of the solvent for 1 to 24 hours to give Compound (XLV).

Compound (XLV) is subjected to a ring-closure reaction using a halogenating agent (e.g. iodine-potassium iodide) and sodium bicarbonate, in a suitable solvent (e.g. THF, diethyl ether, dioxane, water and a mixture thereof) under light-shielding at the refluxing temperature of the solvent for 1 to 24 hours to give Compound (I-ii).

Method 23:

The compound in which R¹³ is hydroxy or an alkoxy may be prepared according to Synthesis, 1989, 275-279 and Tetrahedron Lett., 1984, 25, 4587-4590.

Method 24:

(1) If the compound of the present invention or the starting compound has a functional group (e.g. hydroxyl, amino, carboxyl, etc.) in the above methods, the reaction can proceed by protecting the functional group by a protecting group which is conventionally used in the field of synthetic organic chemistry, and after reaction, the protecting group is removed to give the desired compound. The protecting group for hydroxyl may be tetrahydropyranyl, TMS, and the like. The protecting group for amino may be Boc, benzyloxycarbonyl, etc. The protecting group for carboxy may be an alkyl such as methyl and ethyl, benzyl, etc.

(2) If the compound of the present invention or the starting compound has amino in the above methods, it may be protected if necessary, and then (i) a reaction with an alkyl halide (wherein the alkyl corresponds to “an optionally substituted alkyl” of R⁵ or R⁶) may be performed in the presence of a base (e.g. sodium hydride, triethylamine, sodium carbonate, and potassium carbonate), or (ii) an alcohol (wherein the alkyl moiety corresponds to “an optionally substituted alkyl” of R⁵ or R⁶) may be subjected to Mitsunobu reaction with dialkylazodicarboxylate and triphenylphosphine, and subjected to deprotection if necessary, to give the compound with an amino group which is mono- or di-substituted by an optionally substituted alkyl.

(3) If the compound of the present invention or the starting compound has amino in the above methods, it may be converted into the corresponding amide by a reaction with an acyl halide in a manner similar to the reaction from Compound (I-i) to Compound (I-k) in Method 11.

(4) If the compound of the present invention or the starting compound has carboxy in the above methods, it may be converted into the corresponding carbamoyl by a reaction with an amine in a manner similar to the reaction from Compound (I-m) to Compound (I-o) in Method 12.

(5) If the compound of the present invention or the starting compound has a double bond in the above methods, it may be converted into the corresponding single bond by catalytic hydrogenation using a transition metal (platinum, palladium, rhodium, ruthenium, or nickel) catalyst.

(6) If the compound of the present invention or the starting compound has an ester group in the above methods, it may be converted into the corresponding carboxy by hydrolysis with an alkali (e.g. sodium hydroxide and potassium hydroxide).

(7) If the compound of the present invention or the starting compound has carbamoyl in the above methods, it may be converted into the corresponding nitrile by a reaction with trifluoroacetic anhydride.

(8) If the compound of the present invention or the starting compound has carboxy in the above methods, it may be converted into 4,5-dihydroxazol-2-yl by a reaction with 2-haloethylamine in the presence of a condensation agent.

(9) If the compound of the present invention or the starting compound has hydroxyl in the above methods, it may be converted into the corresponding halogen by treatment with a halogenating agent. Alternatively, if the compound of the present invention or the starting compound has a halogen, it may be converted into the corresponding an alkoxy by treatment with an alcohol.

(10) If the compound of the present invention or the starting compound has an ester in the above methods, it may be converted into the corresponding hydroxyl by reduction with a reducing agent (e.g. a metal reducing agent such as lithium aluminum hydride, sodium borohydride and lithium borohydride; and diborane).

(11) If the compound of the present invention or the starting compound has hydroxyl in the above methods, it may be converted into aldehyde, ketone or carboxy by oxidation with an oxidizing agent (the same as the oxidizing agent mentioned in Method 15).

(12) If the compound of the present invention or the starting compound has carbonyl or aldehyde in the above methods, it may be converted into an aminomethyl which may be mono- or di-substituted by a reductive amination reaction with an amine compound in the presence of a reducing agent (e.g. sodium borohydride and sodium cyanoborohydride).

(13) If the compound of the present invention or the starting compound has carbonyl or aldehyde in the above methods, it may be converted into a double bond by subjecting the compound to Wittig reaction.

(14) If the compound of the present invention or the starting compound has sulfonamide in the above methods, it may be converted into the corresponding sulfonamide salt (e.g. a sodium salt and a potassium salt) by treatment with an alkali (e.g. sodium hydroxide and potassium hydroxide) in an alcohol (e.g. methanol and ethanol).

(15) If the compound of the present invention or the starting compound has an aldehyde in the above methods, it may be converted into the corresponding oxime by a reaction with hydroxylamine or O-alkylhydroxylamine in the presence of a base (e.g. sodium bicarbonate) in an alcohol (e.g. methanol and ethanol).

(16) If the compound of the present invention or the starting compound has a halogen in the above methods, it may be converted into the corresponding cyano group by treatment with a cyanizing agent (the same as the cyanizing agent mentioned in Method 12).

(17) If the compound of the present invention or the starting compound has a halogen in the above methods, it may be converted into the corresponding amine according to the method described in Tetrahedron, pp. 2041-2075, 2002.

(18) If the compound of the present invention or the starting compound has an alkoxycarbonyl in the above methods, it may be converted into the corresponding carbamoyl by condensing the compound with N-hydroxysuccinimide to give a N-succinimidyl ester, and then reacting it with an amine compound. Alternatively, the N-succinimidyl ester may be treated with a reducing agent (e.g. sodium borohydride) to convert the same into the corresponding hydroxymethyl.

(19) If the compound of the present invention or the starting compound has a benzylamine in the above methods, it may be converted into the corresponding amine according to Synthesis, 1985, 770-773.

In the above preparation methods, each of the prepared compounds or intermediates may be purified by a conventional method such as column chromatography and recrystallization. Examples of the recrystallization solvent include an alcohol solvent such as methanol, ethanol and 2-propanol, an ether solvent such as diethyl ether, an ester solvent such as ethyl acetate, an aromatic solvent such as toluene, a ketone solvent such as acetone, a hydrocarbon solvent such as hexane, water, and a mixed solvent thereof. According to a conventional method, the compound of the present invention may also be converted into a pharmaceutically acceptable salt, which may then be subjected to recrystallization, and the like.

The compound (I) of the present invention or the pharmaceutically acceptable salt thereof may be prepared into a pharmaceutical composition comprising a therapeutically effective amount of the compound and a pharmaceutically acceptable carrier. As the pharmaceutically acceptable carrier, there may be mentioned, a diluent, a binder (e.g. syrup, Gum Arabic, gelatin, sorbit, tragacanth and polyvinyl pyrrolidone), an excipient (e.g. lactose, sugar, corn starch, potassium phosphate, sorbit and glycine), a lubricant (e.g. magnesium stearate, talc, polyethylene glycol and silica), a disintegrator (e.g. potato starch) and a humectant (e.g. sodium lauryl sulfate).

The Compound (I) of the present invention or a pharmaceutically acceptable salt thereof can be administered orally or parenterally, and used as suitable pharmaceutical preparations. As the suitable pharmaceutical preparation for oral administration, there are mentioned solid preparations such as tablets, granules, capsules and powders, or liquid preparations such as solutions, suspensions and emulsions. As the suitable pharmaceutical preparation for parenteral administration, there are mentioned a suppository, an injection or a drip infusion using distilled water for injection, physiological saline, an aqueous glucose solution, or an inhalant.

A dose of the compound (I) of the present invention or a pharmaceutically acceptable salt thereof may vary depending on an administration route, an age, weight and condition of a patient, or a kind or degree of a disease, and may be generally about 0.1 to 50 mg/kg per day, more preferably about 0.1 to 30 mg/kg per day.

The compound (I) of the present invention or a pharmaceutically acceptable salt thereof has an excellent large conductance calcium-activated K channel opening activity and hyperpolarizes a membrane electric potential of cells, and is useful for the prophylactic, relief and/or treatment for, for example, hypertension, premature birth, irritable bowel syndrome, chronic heart failure, angina, cardiac infarction, cerebral infarction, subarachnoid hemorrhage, cerebral vasospasm, cerebral hypoxia, peripheral blood vessel disorder, anxiety, male-pattern baldness, erectile dysfunction, diabetes, diabetic peripheral nerve disorder, other diabetic complication, sterility, urolithiasis and pain accompanied thereby, pollakiuria, urinary incontinence, nocturnal enuresis, asthma, chronic obstructive pulmonary diseases (COPD), cough accompanied by asthma or COPD, cerebral apoplexy, cerebral ischemia, traumatic encephalopathy, etc.

### BEST MODE FOR CARRYING OUT THE INVENTION

In the following, the present invention will be explained in more detail by referring to Examples, Reference Examples, but the present invention is not limited by these.

The abbreviations used in the Examples and the Reference Examples each have the meanings as shown below:
THF: tetrahydrofuran
DMF: N,N-dimethylformamide
DMSO: dimethyl sulfoxide
DME: 1,2-dimethoxyethane
Me: methyl
Et: ethyl
t-Bu: tert-butyl
TMS: trimethylsilyl
Tf: trifluoromethanesulfonyl
Boc: tert-butoxycarbonyl
Bn: benzyl
Ph: phenyl

### Example 1

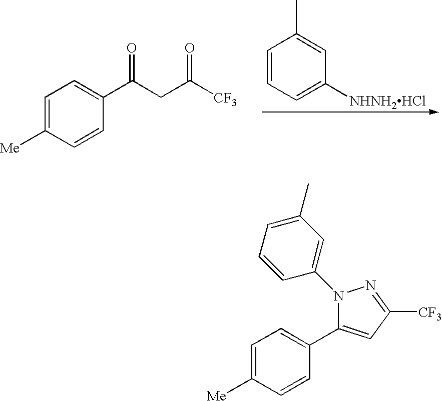

A solution of 4,4,4-trifluoro-1-(4-methylphenyl)butane-1,3-dione (230 mg, 1.00 mmol) and 3-methylphenylhydrazine hydrochloride (174 mg, 1.10 mmol) in ethanol (5 ml) was refluxed under heating for 20 hours. After cooling the reaction mixture, it was concentrated under reduced pressure. The residue was purified by silica gel column chromatography (hexane:ethyl acetate=95:5->90:10) to give 1-(3-methylphenyl)-5-(4-methylphenyl)-3-(trifluoromethyl)-1H-pyrazole (298 mg, 94%) as powders.

MS: 317 [M+H]⁺, APCI (MeOH)

### Examples 2-6

The following compounds were prepared by carrying out a reaction and a treatment in a manner similar to Example 1.

TABLE 1

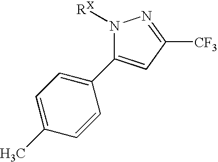

| | | Physical |
|---|---|---|
| Example | R^{x} | constant, etc. |
| 2 | 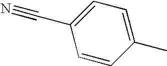 | MS: 328 [M + H]⁺, APCI (MeOH) |
| 3 | 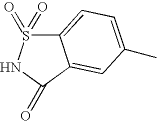 | MS: 408 [M + H]⁺, APCI (MeOH) |
| 4 | 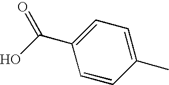 | MS: 345 [M − H]⁻, ESI (MeOH) |
| 5 | 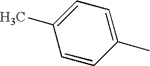 | MS: 317 [M + H]⁺, APCI (MeOH) |
| 6 | 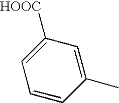 | MS: 345 [M − H]⁻, ESI (MeOH) |
| 7 | 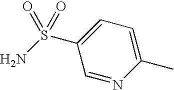 | MS: 383 [M + H]⁺, APCI (MeOH) |

### Example 8

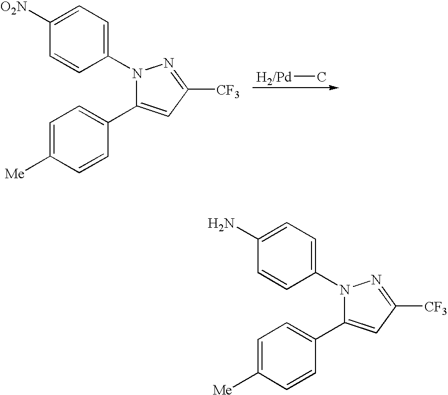

To a solution of 5-(4-methylphenyl)-1-(4-nitrophenyl)-3-(trifluoromethyl)-1H-pyrazole (2.40 g, 6.91 mmol) in THF (50 ml) and ethanol (50 ml) was added 10% Palladium-carbon (250 mg), and the mixture was stirred at room temperature under a hydrogen atmosphere for 2 hours. The insolubles were separated by filtration and were washed with THF, and then, the filtrate and the washing solution were combined and concentrated under reduced pressure to give {4-[5-(4-methylphenyl)-3-(trifluoromethyl)-1H-pyrazol-1-yl]phenyl}-amine (2.14 g, 98%) as a solid.

MS: 318 [M+H]⁺, APCI (MeOH)

### Example 9

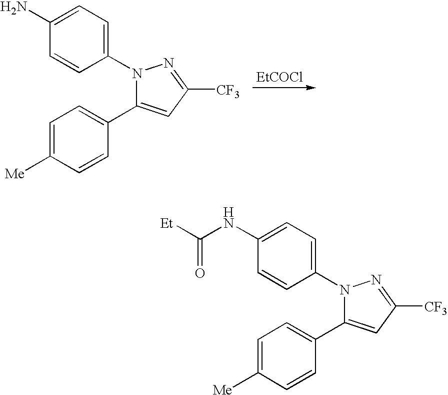

To a solution of {4-[5-(4-methylphenyl)-3-(trifluoromethyl)-1H-pyrazol-1-yl]phenyl}amine (101 mg, 0.32 mmol) and triethylamine (0.066 ml, 0.47 mmol) in methylene chloride (5 ml) was added dropwise propionyl chloride (0.030 ml, 0.35 mmol), and the mixture was stirred at room temperature for 3 days. To the reaction mixture was added diluted hydrochloric acid, and the mixture was extracted with chloroform. The extract was washed with brine and dried over sodium sulfate and the solvent was removed under reduced pressure. The residue was purified by silica gel column chromatography (ethyl acetate:hexane 90:10->80:20) to give N-{4-[5-(4-methylphenyl)-3-(trifluoromethyl)-1H-pyrazol-1-yl]phenyl}propanamide (92 mg, 77%) as powders.

MS: 374 [M+H]⁺, APCI (MeOH)

### Example 10

The following compound was prepared by carrying out a reaction and a treatment in a manner similar to Example 9.

TABLE 2

| | | Physical |
|---|---|---|
| Example | Chemical structure | constant, etc. |
| 10 | 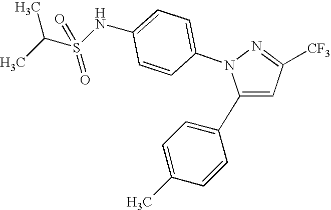 | MS: 425 [M + H]⁺, APCI (MeOH) |

### Example 11

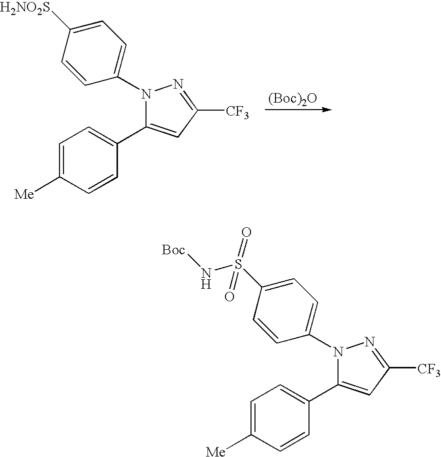

To a suspension of 4-[5-(4-methylphenyl)-3-(trifluoromethyl)-1H-pyrazol-1-yl]benzenesulfonamide (6.93 g, 18.2 mmol) in dichloromethane (70 ml) were added dimethylaminopyridine (0.22 g, 1.82 mmol) and triethylamine (3.80 ml, 27.3 mmol) at room temperature. Thereto was added dropwise a solution of di-tert-butyl dicarbonate (4.76 g, 21.8 mmol) in dichloromethane (70 ml) at room temperature, and the mixture was stirred overnight. The reaction mixture was concentrated under reduced pressure, and ethyl acetate and a 20% aqueous oxalic acid solution were added thereto, and the organic layer was separated. The organic layer was washed with water twice and washed with brine, dried over sodium sulfate and concentrated under reduced pressure. The residue was purified by silica gel column chromatography (hexane:ethyl acetate=4:1->3:1) to give tert butyl({4-[5-(4-methylphenyl)-3-(trifluoromethyl)-1H-pyrazol-1-yl]phenyl}sulfonyl)carbamate (7.64 g, 87%) as powders.

MS: 499 [M+NH₄]⁺, APCI (10 mM-AcONH₄/MeOH)

### Example 12

The following compound was prepared by carrying out a reaction and a treatment in a manner similar to Example 11.

TABLE 3

| Exam- | | Physical constant, |
|---|---|---|
| ple | Chemical structure | etc. |
| 12 | 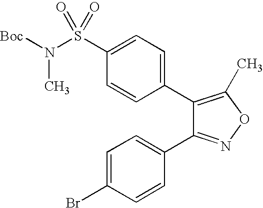 | MS: 524/526 [M + NH₄]⁺, APCI (10 mM- AcONH₄/MeOH) |

### Example 13

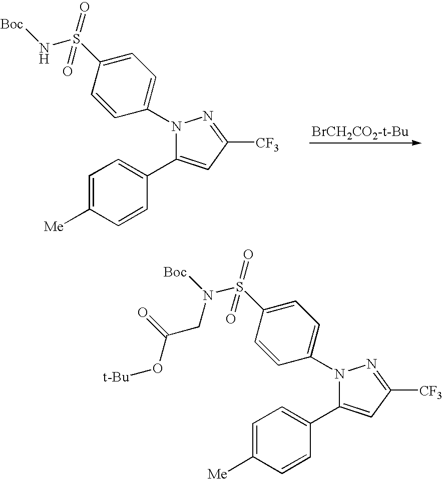

Potassium carbonate (949 mg, 6.87 mmol) was added to a solution of tert-butyl({4-[5-(4-methylphenyl)-3-(trifluoromethyl) -1H-pyrazol-1-yl]phenyl}sulfonyl)carbamate (661 mg, 1.37 mmol) in DMF (3 ml) at room temperature, tert-butyl bromoacetate (321 mg, 1.65 mmol) was added thereto at room temperature, and the mixture was stirred for 5 hours. The reaction mixture was poured into water and extracted with ethyl acetate, and then, the extract was washed with water and brine. It was dried over sodium sulfate and concentrated under reduced pressure. The residue was purified by silica gel column chromatography (hexane:ethyl acetate=12:1) to give tert-butyl N-(tertbutoxycarbonyl)-N-({4-[5-(4-methylphenyl)-3-(trifluoromethyl)-1H-pyrazol-1-yl]phenyl}sulfonyl)glycinate (441 mg, 54%) as powders.

MS: 613 [M+NH₄]⁺, APCI (10 mM-AcONH₄/MeOH)

### Examples 14-21

The following compounds were prepared by carrying out a reaction and a treatment in a manner similar to Example 13.

TABLE 4

| | | Physical |
|---|---|---|
| Example | Chemical structure | constant, etc. |
| 14 | 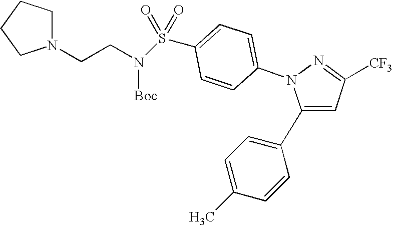 | MS: 579 [M + H]⁺, APCI (10 mM- AcONH₄/MeOH) |

TABLE 5

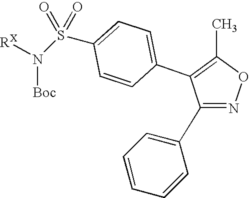

| Example | R^{x} | Physical constant, etc. |
|---|---|---|
| 15 | 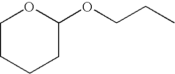 | MS: 560 [M + NH₄]⁺, APCI (10 mM- AcONH₄/MeOH) |
| 16 | 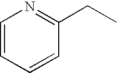 | MS: 506 [M + H]⁺, APCI (10 mM- AcONH₄/MeOH) |
| 17 | 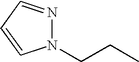 | MS: 509 [M + H]⁺, APCI (10 mM- AcONH₄/MeOH) |
| 18 | 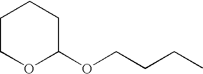 | MS: 574 [M + NH₄]⁺, APCI (10 mM- AcONH₄/MeOH) |
| 19 | 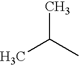 | MS: 474 [M + NH₄]⁺, APCI (10 mM- AcONH₄/MeOH) |
| 20 | 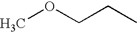 | MS: 490 [M + NH₄]⁺, APCI (10 mM- AcONH₄/MeOH) |
| 21 | 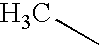 | MS: 446 [M + NH₄]⁺, APCI (10 mM- AcONH₄/MeOH) |

### Example 22

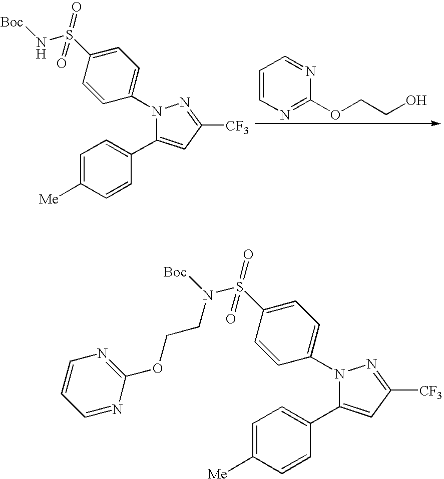

Triphenylphosphine (131 mg, 0.50 mmol) and 2-(2-pyrimidinyloxy)ethanol (70 mg, 0.50 mmol) were added to a solution of tert-butyl({4-[5-(4-methylphenyl)-3-(trifluoromethyl) -1H-pyrazol-1-yl]phenyl}sulfonyl)carbamate (200 mg, 0.42 mmol) in THF (3 ml) at room temperature, and diethyl azodicarboxylate (87 mg, 0.50 mmol) was slowly added-dropwise thereto at room temperature. The reaction mixture was stirred at room temperature overnight, and it was concentrated under reduced pressure. The residue was purified by silica gel column chromatography (hexane:ethyl acetate=90:10->50:50) to give tert-butyl({4-[5-(4-methylphenyl)-3-(trifluoromethyl)-1H-pyrazol-1-yl]phenyl}-sulfonyl)[2-(pyrimidin-2-yloxy)ethyl]carbamate (128 mg, 51%) as a liquid.

MS: 604 [M+H]⁺, APCI (10 mM-AcONH₄/MeOH)

### Examples 23-28

The following compounds were prepared by carrying out a reaction and a treatment in a manner similar to Example 22.

TABLE 6

| | | Physical constant, |
|---|---|---|
| Example | Chemical structure | etc. |
| 23 | 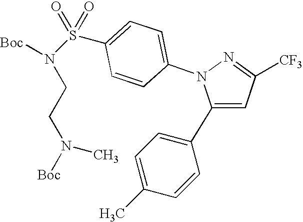 | MS: 661 [M + Na]⁺, ESI (MeOH) |
| 24 | 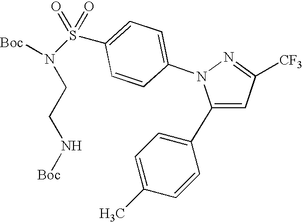 | MS: 647 [M + Na]⁺, ESI (MeOH) |
| 25 | 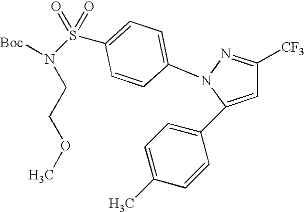 | MS: 557 [M + NH₄]⁺, APCI (10 mM- AcONH₄/MeOH) |
| 26 | 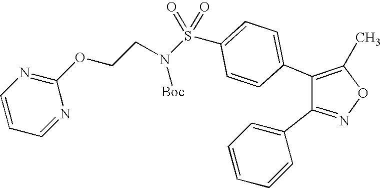 | MS: 537 [M + H]⁺, APCI (10 mM- AcONH₄/MeOH) |
| 27 | 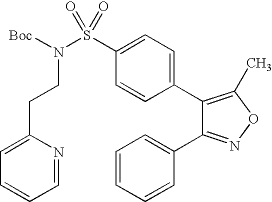 | MS: 520 [M + H]⁺, APCI (10 mM- AcONH₄/MeOH) |
| 28 | 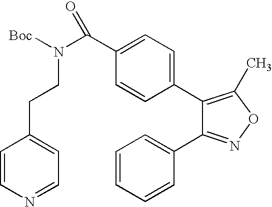 | MS: 498 [M + H]⁺, APCI |

### Example 29

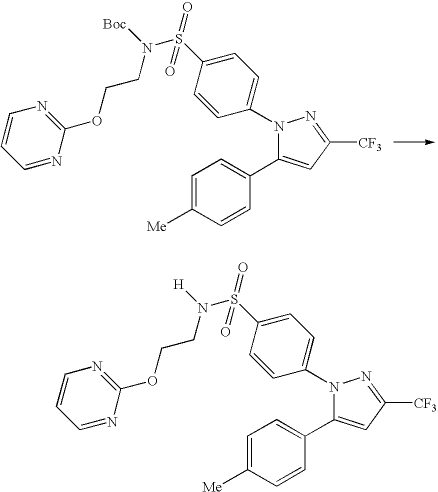

tert-Butyl({4-[5-(4-methylphenyl)-3-(trifluoromethyl)-1H-pyrazol-1-yl]phenyl}sulfonyl)[2-(pyrimidin-2-yloxy)-ethyl]carbamate (150 mg, 0.25 mmol) was dissolved in trifluoroacetic acid (3 ml). The mixture was stirred at room temperature for 2 days and poured into a saturated aqueous sodium bicarbonate solution. The mixture was extracted with ethyl acetate and the extract was washed with brine, and then, it was concentrated under reduced pressure. The residue was purified by silica gel column chromatography (hexane:ethyl acetate=80:20->0:100) to give 4-[5-(4-methylphenyl)-3-(trifluoromethyl)-1H-pyrazol-1-yl]-N-[2-(pyrimidin-2-yloxy)ethyl]benzenesulfonamide (31 mg, 25%) as a liquid.

MS: 504 [M+H]⁺, APCI (MeOH)

### Examples 30-42

The following compounds were prepared by carrying out a reaction and a treatment in a manner similar to Example 29.

TABLE 7

| | | Physical |
|---|---|---|
| Example | Chemical structure | constant, etc. |
| 30 | 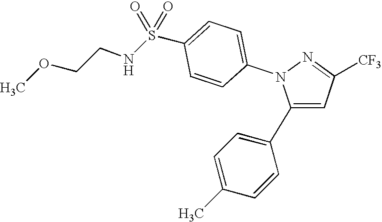 | MS: 440 [M + H]⁺, APCI (MeOH) |
| 31 | 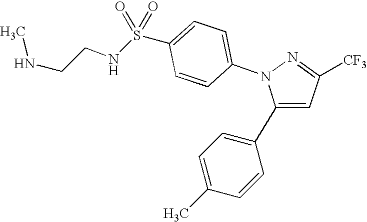 | MS: 439 [M + H]⁺, APCI (MeOH) |
| 32 | 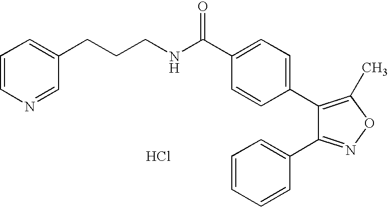 | MS: 398 [M + H]⁺, APCI |
| 33 | 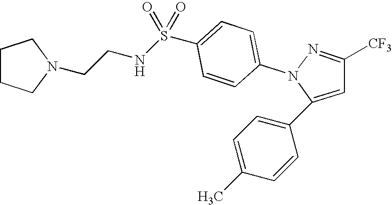 | MS: 479 [M + H]⁺, APCI (MeOH) |
| 34 | 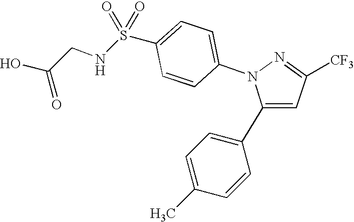 | MS: 438 [M − H]⁻, ESI (MeOH) |
| 35 | 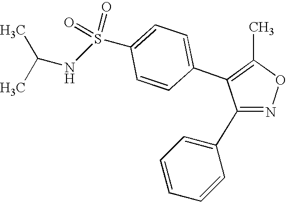 | MS: 357 [M + H]⁺, APCI (MeOH) |
| 36 | 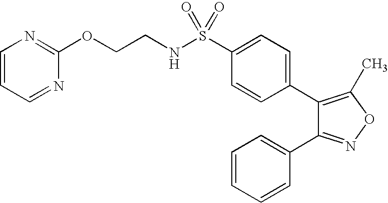 | MS: 437 [M + H]⁺, APCI (MeOH) |
| 37 | 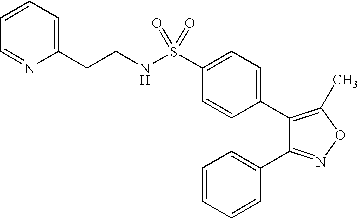 | MS: 420 [M + H]⁺, APCI (MeOH) |
| 38 | 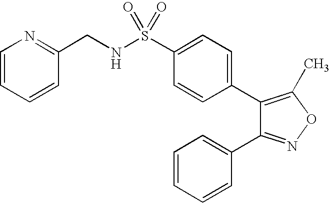 | MS: 406 [M + H]⁺, APCI (MeOH) |
| 39 | 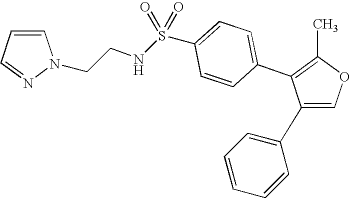 | MS: 409 [M + H]⁺, APCI (MeOH) |
| 40 | 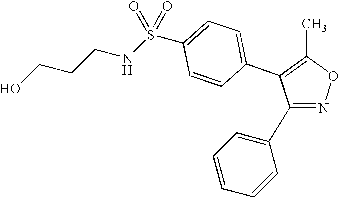 | MS: 373 [M + H]⁺, APCI (MeOH) |
| 41 | 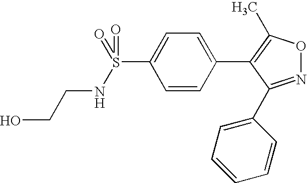 | MS: 357 [M − H]⁻, ESI (MeOH) |
| 42 | 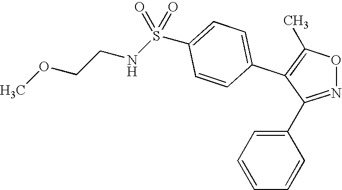 | MS: 371 [M − H]⁻, ESI (MeOH) |

### Example 43

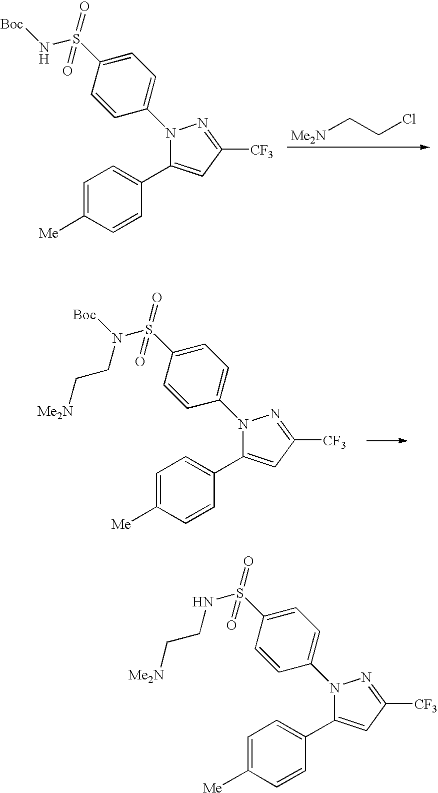

The reaction was carried out in a manner similar to Example 13 using tert-butyl({4-[5-(4-methylphenyl)-3-(trifluoromethyl)-1H-pyrazol-1-yl]phenyl}sulfonyl)carbamate (130 mg, 0.27 mmol) and dimethylaminoethyl chloride hydrochloride (58 mg, 0.40 mmol) to give a crude product, tert-butyl[2-(dimethylamino)ethyl]({4-[5-(4-methylphenyl)-3-(trifluoromethyl)-1H-pyrazol-1-yl]-phenyl}sulfonyl)carbamate. Without isolating the obtained crude product, the reaction was subsequently carried out in a manner similar to Example 28, and the reaction mixture was poured into a saturated aqueous sodium bicarbonate solution and was extracted with ethyl acetate. The extract was washed with water and brine, dried over sodium sulfate and concentrated under reduced pressure. The residue was purified by silica gel column chromatography (chloroform:methanol=50:1). The obtained product was dissolved in a hydrochloric acid-dioxane solution, diethyl ether was added thereto and the mixture was stirred. The precipitated solid was collected by filtration to give N-[2-(dimethylamino)ethyl]-4-[5-(4-methylphenyl)-3-(trifluoromethyl)-1H-pyrazol-1-yl]benzenesulfonamide hydrochloride (98 mg, 74%) as a solid.

MS: 453 [M+H]⁺, AFCI (10 mM-AcONH₄/MeOH)

### Example 44

(1)

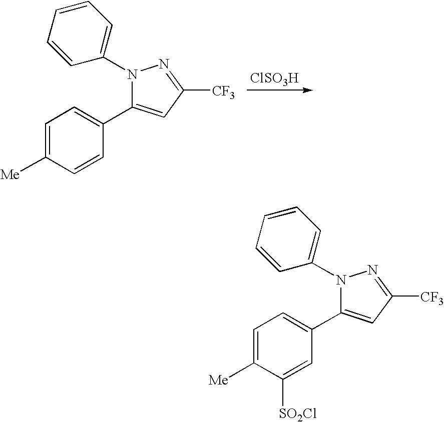

Chlorosulfonic acid (4.36 ml, 65.5 mmol) was added to a solution of 5-(4-methylphenyl)-1-phenyl-3-(trifluoromethyl)-1H-pyrazole (0.99 g, 3.3 mmol) in chloroform (5.0 ml) at room temperature, and the mixture was stirred for 24 hours. The reaction mixture was poured into an ice-water and extracted with chloroform. The organic layer was washed with water, and concentrated. The residue was purified by silica gel column chromatography (hexane:ethyl acetate=100:0->80:20) to give 2-methyl-5-[1-phenyl-3-(trifluoromethyl)-1H-pyrazol-5-yl]benzenesulfonyl chloride (1.17 g, 89%) as powders.

MS: 401/403 [M+H]⁺, APCI (MeOH)

(2)

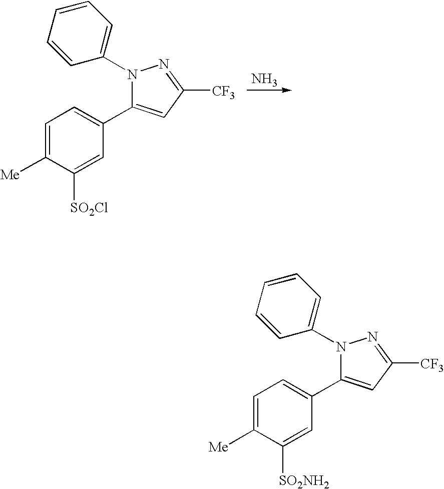

A 30-% aqueous ammonia (2 ml) was added to a solution of 2-methyl-5-[1-phenyl-3-(trifluoromethyl)-1H-pyrazol-5-yl]benzenesulfonyl chloride (100 mg, 0.25 mmol) in THF (5.0 ml) under ice-cooling. The mixture was stirred at the same temperature for 4 hours, and the reaction mixture was poured into ethyl acetate/water. The organic layer was washed with brine, dried over sodium sulfate and concentrated under reduced pressure. The residue was purified by silica gel column chromatography (hexane:ethyl acetate=90:10->0:100) to give 2-methyl-5-[1-phenyl-3-(trifluoromethyl)-1H-pyrazol-5-yl]benzenesulfonamide (86.0 mg, 90%) as powders.

MS: 382 [M+H]⁺, APCI (MeOH)

### Example 45

The following compound was prepared by reacting and treating the compound of Example 1 in a manner similar to Example 44 (1) and (2).

TABLE 8

| Exam- | | Physical |
|---|---|---|
| ple | Chemical structure | constant, etc. |
| 45 | 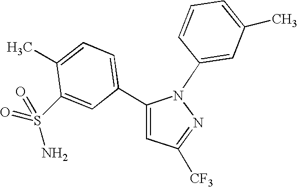 | MS: 396 [M + H]⁺, APCI (MeOH) |

### Example 46

The following compound was prepared by reacting and treating the compound of Example 44 (1) in a manner similar to Example 44 (2).

TABLE 9

| | | Physical |
|---|---|---|
| Example | Chemical structure | constant, etc. |
| 46 | 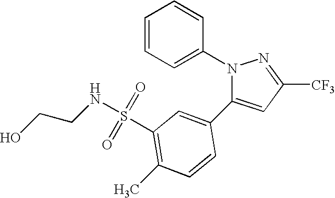 | MS: 426 [M + H]⁺, ESI |

### Example 47

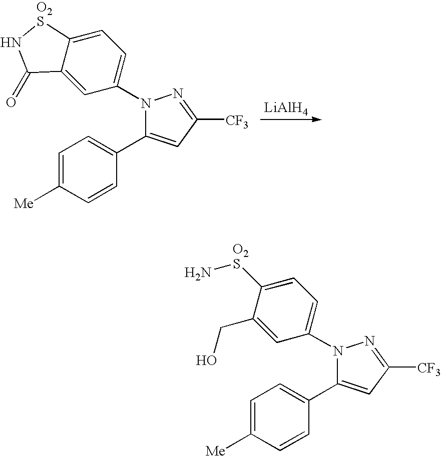

5-[5-(4-Methylphenyl)-3-(trifluoromethyl)-1H-pyrazol-1-yl]-1,2-benzoisothiazol-3(2H)-one 1,1-dioxide (160 mg, 0.4 mmol) was added to a suspension of lithium aluminum hydride (53.2 mg, 1.4 mmol) in THF (3 ml) at −78° C. The reaction mixture was warmed to room temperature, and then, the mixture was stirred for 4 hours. To the reaction mixture were added ice, a 10% aqueous hydrochloric acid solution and ethyl acetate, and the organic layer was separated. The organic layer was washed with water, dried over sodium sulfate, and concentrated under reduced pressure. The residue was purified by silica gel column chromatography (hexane:ethyl acetate=70:30->50:50) to give 2-(hydroxymethyl)-4-[5-(4-methylphenyl)-3-(trifluoromethyl) -1H-pyrazol-1-yl]benzenesulfonamide (77 mg, 47%) as a solid.

MS: 412 [M+H]⁺, APCI (MeOH)

### Example 48

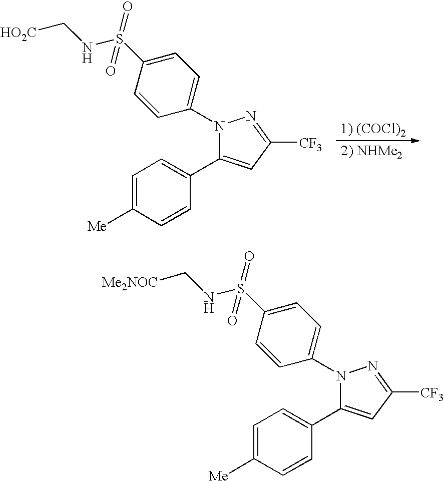

Oxalyl chloride (23 mg, 0.18 mmol) and one drop of DMF were added to a suspension of N-({4-[5-(4-methylphenyl)-3-(trifluoromethyl)-1H-pyrazol-1-yl]phenyl}sulfonyl)glycine (60 mg, 0.14 mmol) in dichloromethane (2 ml), and the mixture was stirred for 3 hours. The reaction mixture was concentrated under reduced pressure, the residue was dissolved in THF (2 ml), and then, the mixture was added to a 50% aqueous dimethylamine solution (2 ml)/ethyl acetate (2 ml) under ice-cooling with stirring. The mixture was stirred at the same temperature for 2 hours, and poured into ethyl acetate/water. The organic layer was washed with brine, dried over sodium sulfate, and concentrated under reduced pressure. The residue was purified by silica gel column chromatography (hexane:ethyl acetate=80:20->0:100) to give N,N-dimethyl-2-({4-[5-(4-methylphenyl)-3-(trifluoromethyl)-1H-pyrazol-1-yl]-phenyl}sulfonyl)acetamide (55 mg, 86%) as a liquid.

MS: 467 [M+H]⁺, APCI (MeOH)

### Example 49

The following compound was prepared by reacting and treating the compound of Example 4 in a manner similar to Example 48.

TABLE 10

| | | Physical |
|---|---|---|
| Example | Chemical structure | constant, etc. |
| 49 | 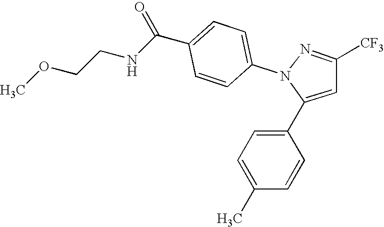 | MS: 404 [M + H]⁺, APCI (MeOH) |

### Examples 50-57

The following compounds were prepared by carrying out a reaction and a treatment in a manner similar to Example 48.

TABLE 11

| | | Physical |
|---|---|---|
| Example | Chemical structure | constant, etc. |
| 50 | 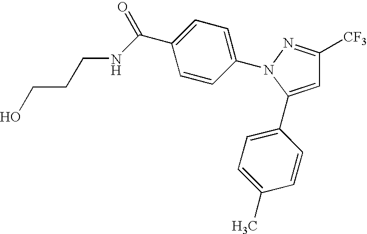 | MS: 404 [M + H]⁺, APCI (MeOH) |
| 51 | 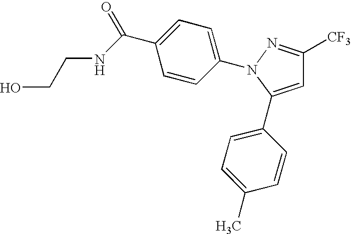 | MS: 390 [M + H]⁺, APCI (MeOH) |
| 52 | 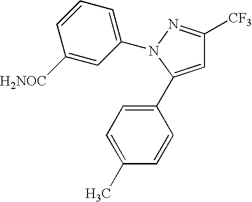 | MS: 346 [M + H]⁺, APCI (MeOH) |
| 53 | 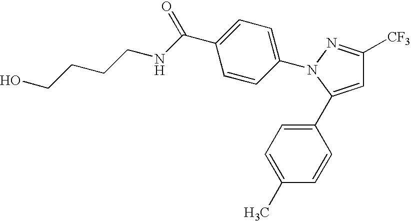 | MS: 418 [M + H]⁺, ESI |
| 54 | 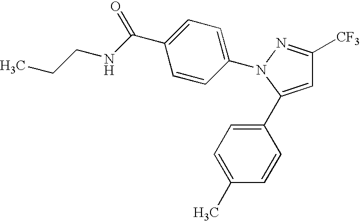 | MS: 388 [M + H]⁺, ESI |
| 55 | 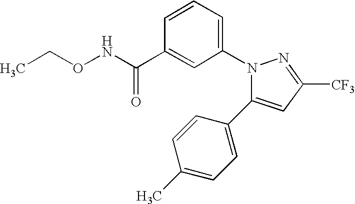 | MS: 390 [M + H]⁺, ESI |
| 56 | 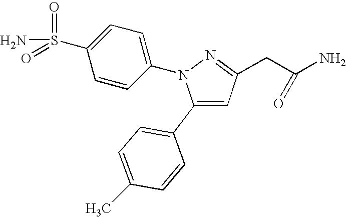 | MS: 371 [M + H]⁺, APCI (MeOH) |
| 57 | 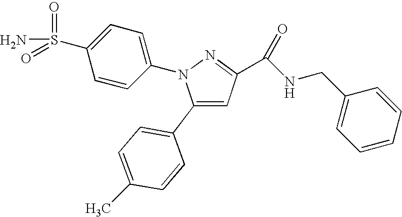 | MS: 447 [M + H]⁺, APCI (MeOH) |

### Example 58

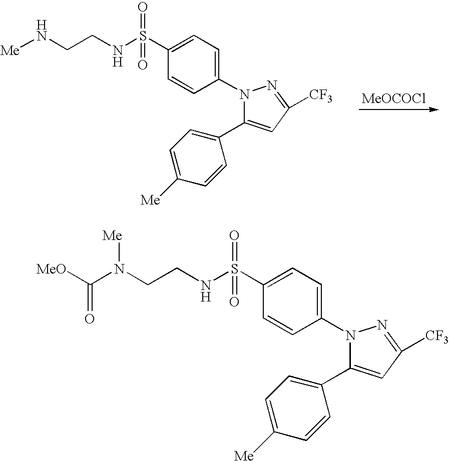

Methyl chlorocarbonate (16 mg, 0.14 mmol) was added to a solution of N-(2-methylaminoethyl)-4-[5-(4-methylphenyl)-3-(trifluoromethyl)-1H-pyrazol-1-yl]benzenesulfonamide (52.0 mg, 0.12 mmol) in pyridine (2 ml) and the mixture was stirred overnight. The reaction mixture was concentrated under reduced pressure and the residue was purified by silica gel column chromatography (hexane:ethyl acetate=80:20->0:100) to give methyl N-methyl-{2-[({4-[5-(4-methylphenyl)-3-(trifluoromethyl)-1H-pyrazol-1-yl]phenyl}-sulfonyl)amino]ethyl}carbamate (50.4 mg, 86%) as a solid.

MS: 497 [M+H]⁺, APCI (MeOH)

### Example 59

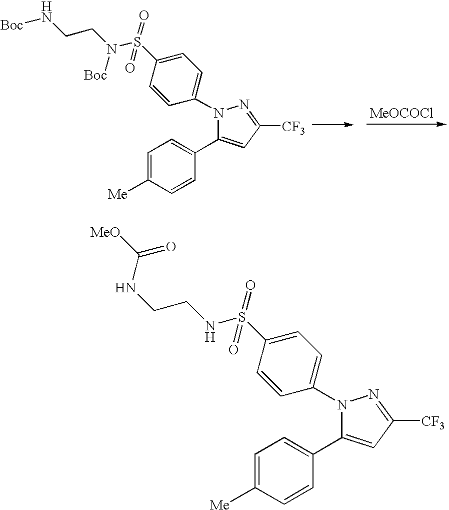

The reaction was carried out in a manner similar to Example 28 using tert-butyl[2-(tert-butoxycarbonylamino)-ethyl] ({4-[5-(4-methylphenyl)-3-(trifluoromethyl)-1H-pyrazol-1-yl]phenyl}sulfonyl)carbamate (87.8 mg, 0.14 mmol). Without isolating the obtained crude product, the reaction was subsequently carried out in a manner similar to Example 58 using methyl chlorocarbonate (16 mg, 0.14 mmol). The reaction mixture was concentrated under reduced pressure, and the residue was purified by silica gel column chromatography (hexane:ethyl acetate=80:20->0:100) to give methyl {2-[({4-[5-(4-methylphenyl)-3-(trifluoromethyl)-1H-pyrazol-1-yl]phenyl}sulfonyl)amino]-ethyl}carbamate (33.5 mg, 50%) as a solid.

MS: 481 [M−H]⁻, ESI(MeOH)

### Example 60

(1)

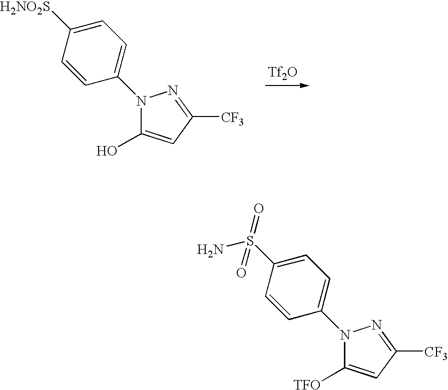

Trifuloromethanesulfonic anhydride (15.5 ml, 92.1 mmol) was added dropwise to a suspension of 4-[5-hydroxy-3-(trifluoromethyl)-1H-pyrazol-1-yl]benzenesulfonamide (23.6 g, 76.7 mmol) and 2,6-di-tert-butyl-4-methylpyridine (24.6 g, 119.9 mmol) in dichloromethane (750 ml) at −20° C. under argon atmosphere. The mixture was warmed to 0° C., stirred at the same temperature for 30 minutes, and then, the reaction mixture was poured into a saturated aqueous sodium bicarbonate solution under ice-cooling. The organic layer was washed with brine, dried over sodium sulfate, and concentrated under reduced pressure to approximately 200 ml. The precipitate was collected by filtration and washed with dichloromethane to give 1-[4-(aminosulfonyl)phenyl]-3-(trifluoromethyl)-1H-pyrazol-5-yl trifluoromethanesulfonate (23.6 g, 70%) as a solid. Melting point: 114-115° C.

(2)

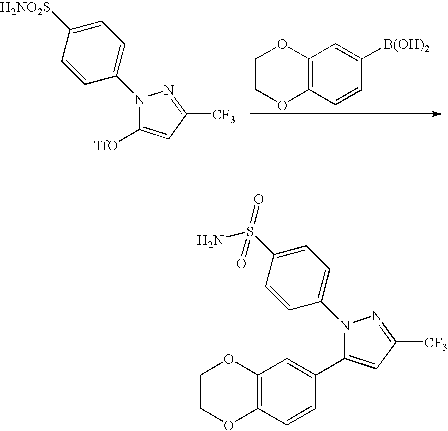

1-[4-(Aminosulfonyl)phenyl]-3-(trifluoromethyl)-1H-pyrazol -5-yl trifluoromethanesulfonate (220 mg, 0.50 mmol), 1,4-benzodioxane-6-boronic acid (108 mg, 0.60 mmol), potassium carbonate (346 mg, 2.50 mmol) and dichlorobis-(triphenylphosphine) palladium (70 mg, 0.10 mmol) were suspended in 1,4-dioxane (3 ml) and the suspension was refluxed under heating for 6 hours. The suspension was poured into ethyl acetate/water, and the organic layer was washed with brine, dried over sodium sulfate, and concentrated under reduced pressure. The residue was purified by silica gel column chromatography (hexane:ethyl acetate=90:10->25:75) and recycle HPLC to give 4-[5-(2,3-dihydro-1,4-benzodioxan-6-yl)-3-(trifluoromethyl)-1H-pyrazol -1-yl]benzenesulfonamide (103 mg, 48%) as a solid.

MS: 426 [M+H]⁺, APCI (MeOH)

### Example 61

(1)

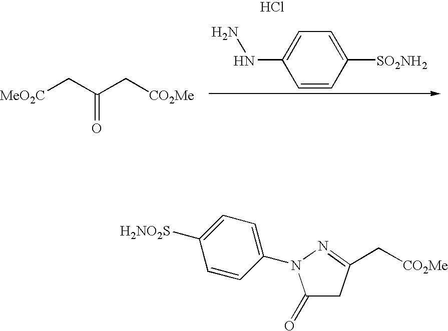

Dimethyl 1,3-acetonedicarboxylate (13.8 g, 79.0 mmol) and 4-sulfonamidophenylhydrazine hydrochloride (17.6 g, 79.0 mmol) were heated while stirring at 100° C. for 2 hours. After cooling the reaction mixture, a saturated aqueous sodium bicarbonate solution was added thereto and the mixture was washed with THF-ethyl acetate. A 10% hydrochloric acid was added to an aqueous layer to adjust pH to 4 and the mixture was extracted with THF-ethyl acetate twice. The extract was dried over magnesium sulfate and concentrated under reduced pressure. After diethyl ether-ethyl acetate was added to the residue and the mixture was stirred, the obtained solid was collected by filtration to give methyl {1-[4-(aminosulfonyl)phenyl]-5-oxo-4,5-dihydro-1H-pyrazol-3-yl}acetate (12.85 g, 52%) as a solid.

MS: 312 [M+H]⁺, APCI (MeOH)

(2) The following compound was prepared by carrying out a reaction and a treatment in a manner similar to Example 60 (1).

TABLE 12

| Example | Chemical structure | Physical constant, etc. |
|---|---|---|
| 61(2) | 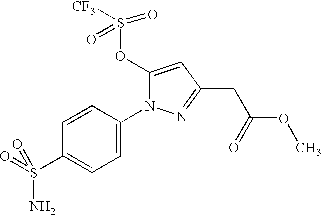 | NMR (CDCl₃): 3.75 (2H, s), 3.76 (3H, s), 4.91 (2H, s), 6.43 (1H, s), 7.76 (2H, d, J = 9.0 Hz), 8.06 (2H, d, J = 9.0 Hz) |

(3) The following compound was prepared by carrying out a reaction and a treatment in a manner similar to Example 60 (2).

TABLE 13

| | | Physical |
|---|---|---|
| Example | Chemical structure | constant, etc. |
| 61(3) | 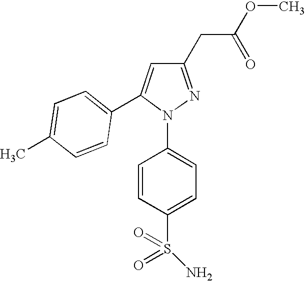 | MS: 386 [M + H]⁺, APCI (MeOH) |

### Example 62

The following compound was prepared by carrying out a reaction and a treatment in a manner similar to Example 61 (1) and Example 60 (1) to (2). The obtained compound was converted to sodium salt according to a conventional method.

TABLE 14

| | Chemical | Physical |
|---|---|---|
| Example | structure | constant, etc. |
| 62(1) | 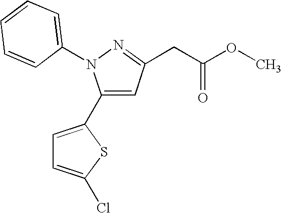 | MS: 333/335 [M + H]⁺, APCI (MeOH) |
| 62(2) | 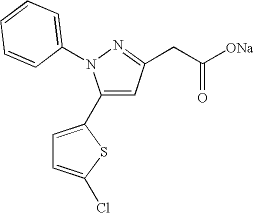 | MS: 317/319 [M − Na]⁻, ESI |

### Examples 63-67

The following compounds were prepared by carrying out a reaction and a treatment in a manner similar to Example 60 (2).

TABLE 15

| | | Physical constant, |
|---|---|---|
| Example | R^{x} | etc. |
| 63 | 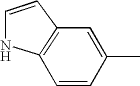 | MS: 407 [M + H]⁺, APCI (MeOH) |
| 64 | 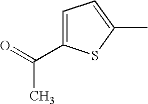 | MS: 416 [M + H]⁺, AFCI (MeOH) |
| 65 | 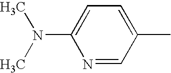 | MS: 412 [M + H)⁺, APCI (MeOH) |
| 66 | 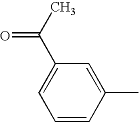 | MS: 410 [M + H]⁺, APCI (MeOH) |

### Example 67

(1) The following compound was prepared by carrying out a reaction and a treatment in a manner similar to Example 62 (1).

TABLE 16

| | | Physical |
|---|---|---|
| Example | Chemical structure | constant, etc. |
| 67(1) | 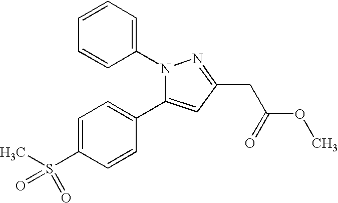 | MS: 371 [M + H]⁺, APCI (MeOH) |

(2) The following compound was prepared by carrying out a reaction and a treatment in a manner similar to Example 62 (2).

TABLE 17

| | | Physical |
|---|---|---|
| Example | Chemical structure | constant, etc. |
| 67(2) | 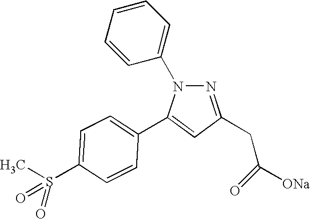 | MS: 355 [M − Na]⁻, ESI (MeOH) |

### Example 68

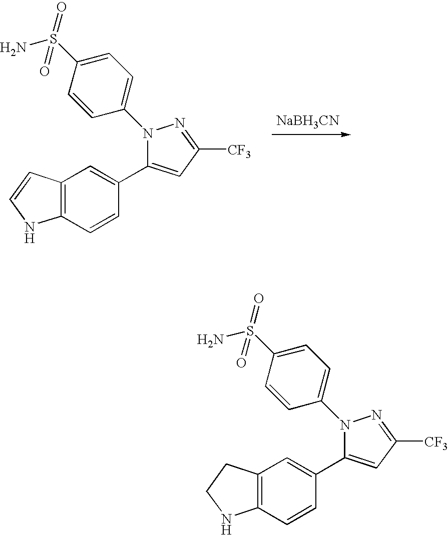

Sodium cyanoborohydride (186 mg, 2.95 mmol) was added to a solution of 4-[5-(1H-indol-5-yl)-3-(trifluoromethyl)-1H-pyrazol -1-yl]benzenesulfonamide (300 mg, 0.74 mmol) in acetic acid (4 ml) at room temperature and the mixture was stirred at room temperature for 4 hours. After the reaction mixture was basified with a saturated aqueous sodium bicarbonate solution under ice-cooling, it was extracted with ethyl acetate. The extract was washed with water and brine. Then, it was dried over sodium sulfate and concentrated under reduced pressure. The residue was purified by silica gel column chromatography (hexane:ethyl acetate=80:20->25:75) to give 4-[5-(2,3-dihydro-1H-indol -5-yl)-3-(trifluoromethyl)-1H-pyrazol-1-yl]benzenesulfonamide (155 mg, 51%) as powders.

MS: 409 [M+H]⁺, APCI (MeOH)

### Example 69

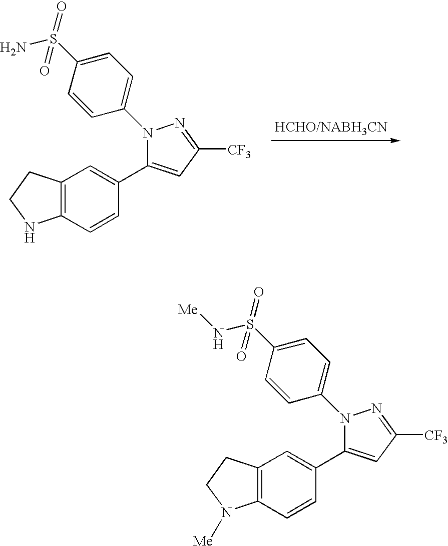

Sodium cyanoborohydride (32 mg, 0.50 mmol) was added to a solution of 4-[5-(2,3-dihydro-1H-indol-5-yl)-3-(trifluoromethyl) -1H-pyrazol-1-yl]benzenesulfonamide (102 mg, 0.25 mmol) in methanol (3 ml) under ice-cooling, and then, pH of the mixture was adjusted to 4 with a 1% aqueous hydrochloric acid solution and an aqueous formalin solution (30%, 1 ml) was added thereto. After the reaction mixture was stirred at room temperature overnight, it was concentrated under reduced pressure. The residue was made basic with 30% aqueous ammonia and extracted with ethyl acetate. The extract was washed with brine, dried over sodium sulfate, and concentrated under reduced pressure. The residue was purified by silica gel column chromatography (hexane:ethyl acetate=90:10->25:75) to give N-methyl -4-[5-(1-methyl-2,3-dihydro-1H-indol-5-yl)-3-(trifluoromethyl) -1H-pyrazol-1-yl]benzenesulfonamide (98 mg, 90%) as powders.

MS: 437 [M+H]⁺, APCI (MeOH)

### Example 70

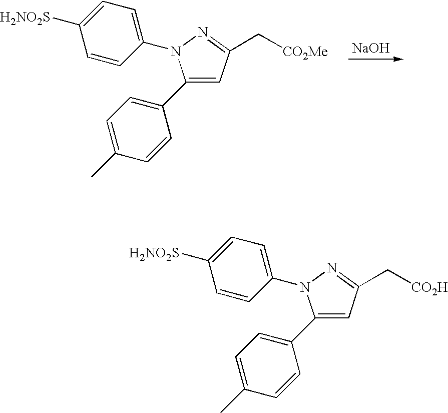

A 2N sodium hydroxide solution (12.8 ml, 25.6 mmol) was added to a solution of methyl[1-[4-(aminosulfonyl)-phenyl]-5-(4-methylphenyl)-1H-pyrazol-3-yl]acetate (3.30 g, 8.56 mmol) in methanol (33 ml) and the mixture was refluxed under heating for 30 minutes. After cooling the reaction mixture, it was concentrated under reduced pressure and 10% hydrochloric acid was added thereto and extracted with ethyl acetate. The organic layer was washed with brine, dried over magnesium sulfate, and concentrated under reduced pressure. After hexane was added to the residue and the mixture was stirred, it was concentrated under reduced pressure to give [1-[4-(aminosulfonyl)phenyl]-5-(4-methylphenyl)-1H-pyrazol-3-yl]acetic acid (2.8 g, 87%) as powders.

MS: 370 [M−H]⁻, ESI

### Example 71

The following compound was prepared by carrying out a reaction and a treatment in a manner similar to Example 70.

TABLE 18

| | | Physical |
|---|---|---|
| Example | Chemical structure | constant, etc. |
| 71 | 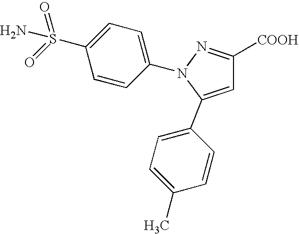 | MS: 356 [M − H]⁻, ESI (MeOH) |

### Example 72

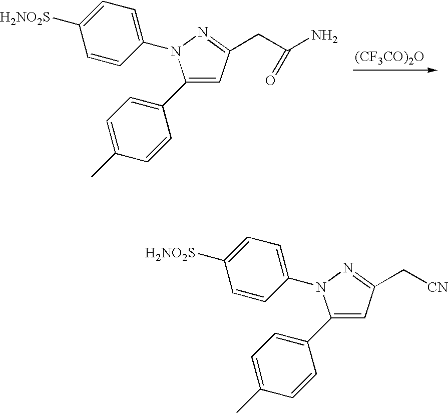

Trifluoroacetic anhydride (143 mg, 0.68 mmol) was added dropwise to a suspension of 2-[1-[4-(aminosulfonyl)phenyl]-5-(4-methylphenyl)-1H-pyrazol-3-yl]acetamide (126 mg, 0.34 mmol) and pyridine (108 mg, 1.36 mmol) in chloroform (4 ml) under ice-cooling, and the mixture was stirred at room temperature overnight. To the reaction mixture was added a 10% aqueous sodium hydroxide solution, and the mixture was stirred for 30 minutes and ethyl acetate/water was added thereto. The organic layer was washed with brine, dried over sodium sulfate, and concentrated under reduced pressure. The residue was purified by silica gel column chromatography (chloroform:methanol=30:1->20:1) to give 4-[3-(cyanomethyl)-5-(4-methylphenyl) -1H-pyrazol-1-yl]benzenesulfonamide (20 mg, 17%) as powders.

MS: 351 [M−H]⁻, ESI (MeOH)

### Example 73

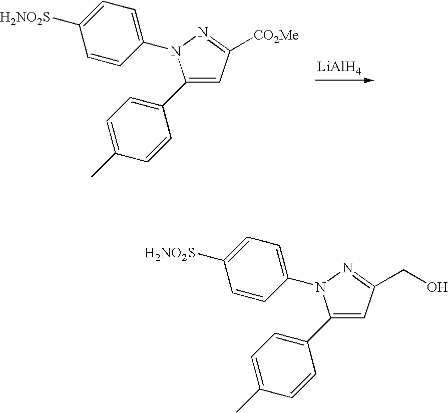

Lithium aluminum hydride (8.54 g, 0.23 mol) was added at several times to a solution of methyl 1-[4-(aminosulfonyl)phenyl]-5-(4-methylphenyl)-1H-pyrazol-3-carboxylate (55.7 g, 0.15 mol) in THF (1.5 liters) at room temperature and the mixture was refluxed under heating for 2 hours. After the reaction mixture was cooled with ice, 10% hydrochloric acid was slowly added thereto. After stirring the mixture, ethyl acetate (500 ml) and water (500 ml) were added thereto and the mixture was portioned. The organic layer was washed with brine, dried over magnesium sulfate, and concentrated under reduced pressure. To the residue was added methanol-diethyl ether-hexane, and the mixture was stirred. The obtained crystals were collected by filtration. They were washed with diethyl ether and hexane and dried to give 4-[3-(hydroxymethyl)-5-(4-methylphenyl)—1H-pyrazol-1-yl]benzenesulfonamide (42.8 g, 83%) as crystal.

Melting point: 173-174° C.

MS: 344 [M+H]⁺, APCI (MeOH)

### Example 74

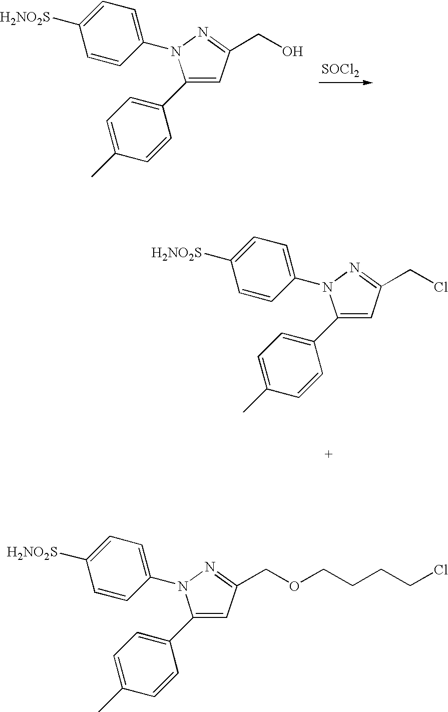

Thionyl chloride (0.33 ml, 4.52 mmol) was added to a solution of 4-[3-(hydroxymethyl)-5-(4-methylphenyl)-1H-pyrazol -1-yl]benzenesulfonamide (1.03 g, 3.00 mmol) in THF (20 ml) and the mixture was refluxed under heating for 1 hour. After the reaction mixture was cooled and concentrated under reduced pressure, the residue was purified by silica gel column chromatography (chloroform:methanol=50:1) to give 4-[3-(chloromethyl)-5-(4-methylphenyl)-1H-pyrazol -1-yl]benzenesulfonamide (242 mg, 38%) and 4-[3-[(4-chlorobutoxy)methyl]-5-(4-methylphenyl)-1H-pyrazol-1-yl]benzenesulfonamide (457 mg, 60%) as powders, respectively.

### 4-[3-(chloromethyl)-5-(4-methylphenyl)-1H-pyrazol-1-yl]benzenesulfonamide

MS: 362/364 [M+H]⁺, APCI (MeOH)

### 4-[3-[(4-chlorobutoxy)methyl]-5-(4-methylphenyl)-1H-pyrazol -1-yl]benzenesulfonamide

MS: 434/436 [M+H]⁺, APCI (MeOH)

### Example 75

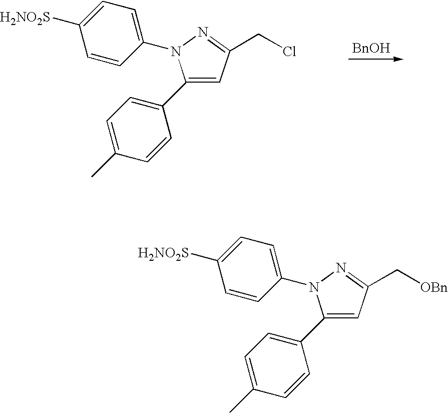

Sodium hydride (60%, 30 mg, 0.75 mmol) was added to a solution of 4-[3-(chloromethyl)-5-(4-methylphenyl)-1H-pyrazol -1-yl]benzenesulfonamide (90 mg, 0.25 mmol) and benzyl alcohol (81 mg, 0.75 mmol) in THF (3 ml) and the mixture was refluxed under heating overnight. After the reaction mixture was cooled, 10% hydrochloric acid was added thereto and the mixture was extracted with ethyl acetate. The extract was washed with brine, dried over sodium sulfate, and concentrated under reduced pressure. The residue was purified by silica gel column chromatography (hexane:ethyl acetate=3:2->1:1) to give 4-[3-[(benzyloxy)methyl]-5-(4-methylphenyl)-1H-pyrazol-1-yl]benzenesulfonamide (37 mg, 33%) as a liquid.

MS: 434 [M+H]⁺, APCI (MeOH)

### Example 76

The following compound was prepared by carrying out a reaction and a treatment in a manner similar to Example 75.

TABLE 19

| | | Physical |
|---|---|---|
| Example | Chemical structure | constant, etc. |
| 76 | 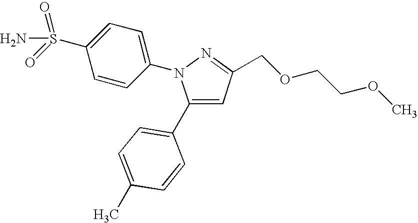 | MS: 402 [M + H]⁺, APCI (MeOH) |

### Example 77

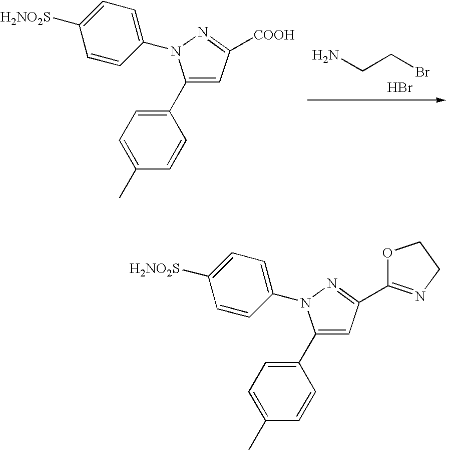

A solution of 1-[4-(aminosulfonyl)phenyl]-5-(4-methylphenyl) -1H-pyrazol-3-carboxylic acid (357 mg, 1.0 mmol), 2-bromoethylamine hydrobromide (287 mg, 1.40 mmol), N-hydroxybenzotriazole (203 mg, 1.50 mmol), triethylamine (0.42 ml, 3.00 mmol) and 1-ethyl-3-(3′-dimethylaminopropyl)carbodiimide hydrochloride (288 mg, 1.50 mmol) in DMF (5 ml) was stirred at room temperature overnight. Water was added to the reaction mixture and the mixture was extracted with ethyl acetate. The extract was washed with brine, dried over sodium sulfate, and concentrated under reduced pressure. The residue was purified by silica gel column chromatography (chloroform:methanol=50:1->30:1) to give 4-[3-(4,5-dihydro-1,3-oxazol-2-yl) -5-(4-methylphenyl)-1H-pyrazol-1-yl]benzenesulfonamide (170 mg, 45%) as powders.

MS: 383 [M+H]⁺, APCI (MeOH)

### Example 78

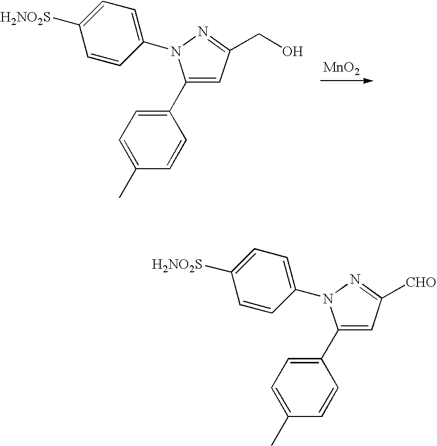

A suspension of 4-[3-(hydroxymethyl)-5-(4-methylphenyl) -1H-pyrazol-1-yl]benzenesulfonamide (7.0 g, 0.020 mol) and manganese dioxide (35 g, 0.10 mol) in THF (140 ml) was refluxed under heating for an hour. After cooling the reaction mixture, the insolubles were removed by filtration and washed with ethyl acetate. The filtrate and the washing solution were combined and concentrated under reduced pressure. The residue was tritulated with diethyl ether to give 4-[3-formyl-5-(4-methylphenyl)-1H-pyrazol -1-yl]benzenesulfonamide (4.8 g, 68%) as powders.

MS: 340 [M−H]⁻, ESI(MeOH)

### Example 79

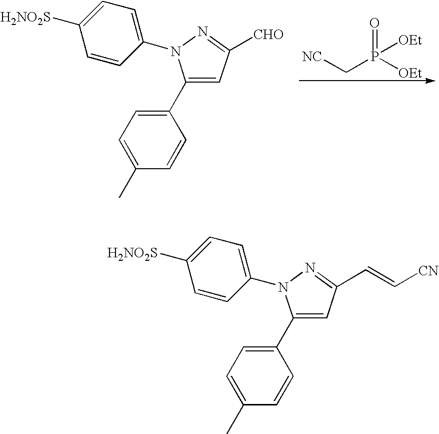

Diethyl(cyanomethyl)phosphonate (0.16 ml, 1.2 mmol) and potassium tert-butoxide (135 mg, 1.2 mmol) were added to a solution of 4-[3-formyl-5-(4-methylphenyl)-1H-pyrazol-1-yl]benzenesulfonamide (341 mg, 1.00 mmol) in THF (4 ml) and the mixture was stirred at room temperature overnight. Water was added to the reaction mixture and the mixture was extracted with ethyl acetate. The extract was washed with brine, dried over sodium sulfate, and concentrated under reduced pressure. The residue was purified by silica gel column chromatography (chloroform:methanol=50:1->30:1) to give 4-[3-[(E)-2-cyanovinyl]-5-(4-methylphenyl)-1H-pyrazol-1-yl]benzenesulfonamide (316 mg, 87%) as powders.

MS: 365 [M+H]⁺, APCI (MeOH)

### Examples 80 and 81

The following compounds were prepared by carrying out a reaction and a treatment in a manner similar to Example 79.

TABLE 20

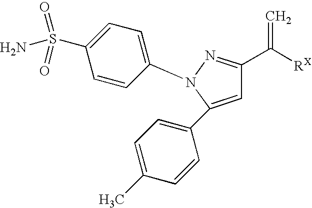

| | | Physical |
|---|---|---|
| Example | R^{x} | constant, etc. |
| 80 | —CH₃ | MS: 354 [M + H]⁺, |
| | | APCI (MeOH) |
| 81 | —H | MS: 340 [M + H]⁺, |
| | | APCI (MeOH) |

### Example 82

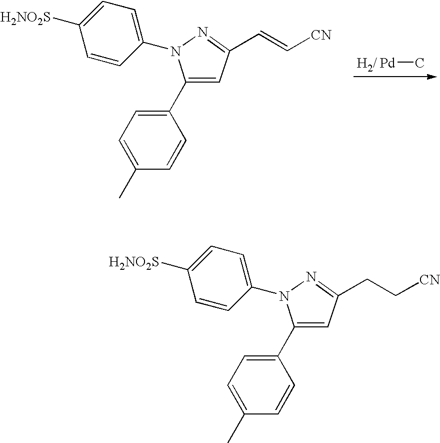

A suspension of 4-[3-[(E)-2-cyanovinyl]-5-(4-methylphenyl) -1H-pyrazol-1-yl]benzenesulfonamide (250 mg, 0.69 mmol) and 5% palladium-carbon (500 mg) in methanol (8 ml) was stirred under hydrogen atmosphere at room temperature overnight. After the insolubles were removed by filtration and washed with methanol, the filtrate and the washing solution were combined and the mixture was concentrated under reduced pressure. The residue was purified by silica gel column chromatography (chloroform:methanol=50:1->30:1) to give 4-[3-(2-cyanoethyl)-5-(4-methylphenyl)-1H-pyrazol-1-yl]benzenesulfonamide (169 mg, 79%) as powders.

MS: 367 [M+H]⁺, APCI (MeOH)

### Example 83

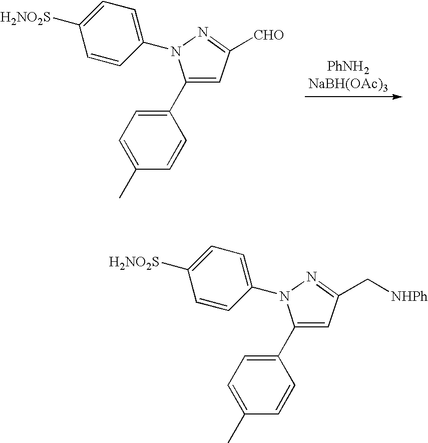

Sodium triacetoxy borohydride (223 mg, 1.0 mmol) was added to a solution of 4-[3-formyl-5-(4-methylphenyl)-1H-pyrazol -1-yl]benzenesulfonamide (171 mg, 0.50 mmol) and aniline (0.055 ml, 0.60 mmol) in THF (4 ml) at room temperature and the mixture was stirred overnight. An aqueous sodium bicarbonate solution was added to the reaction mixture and the mixture was extracted with ethyl acetate. The extract was washed with brine, dried over sodium sulfate, and concentrated under reduced pressure. The residue was purified by silica gel column chromatography (chloroform:methanol=50:1->30:1) to give 4-[3-(anilinomethyl)-5-(4-methylphenyl)-1H-pyrazol-1-yl]-benzenesulfonamide (157 mg, 75%) as powders.

MS: 419 [M+H]⁺, APCI (MeOH)

### Example 84

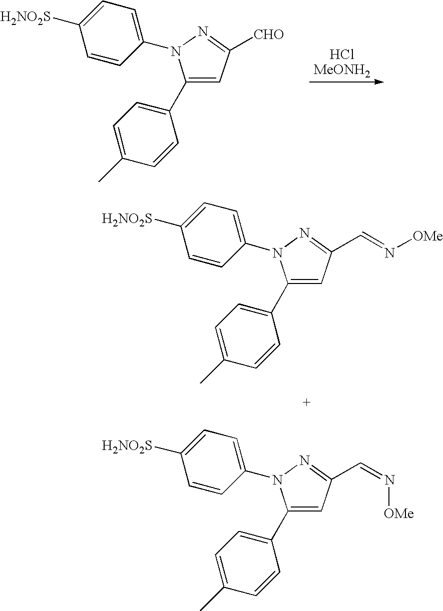

A mixture of 4-[3-formyl-5-(4-methylphenyl)-1H-pyrazol-1-yl]benzenesulfonamide (341 mg, 1.0 mmol), O-methylhydroxylamine hydrochloride (125 mg, 1.5 mmol) and sodium carbonate (79 mg, 0.75 mmol) in ethanol (3 ml) and water (3 ml) was refluxed under heating for 3 hours. Water was added to the reaction mixture and the mixture was extracted with ethyl acetate. The extract was washed with brine, dried over anhydrous sodium sulfate, and concentrated under reduced pressure. The residue was purified by silica gel column chromatography (chloroform:methanol=50:1->30:1) to give 4-[3-[(E)-(methoxyimino)methyl]-5-(4-methylphenyl)-1H-pyrazol-1-yl]benzenesulfonamide (trans; 280 mg, 75%) as powders, and 4-[3-[(Z)-(methoxyimino)methyl]-5-(4-methylphenyl)-1H-pyrazol-1-yl]benzenesulfonamide (cis; 93 mg, 25%) as a solid.

### 4-[3-[(E)-(methoxyimino)methyl]-5-(4-methylphenyl)-1H-pyrazol -1-yl]benzenesulfonamide

MS: 371 [M+H]⁺, APCI (MeOH)

### 4-[3-[(Z)-(methoxyimino)methyl]-5-(4-methylphenyl)-1H-pyrazol -1-yl]benzenesulfonamide

MS: 371 [M+H]⁺, APCI (MeOH)

### Example 85

The following compound was prepared by carrying out a reaction and a treatment in a manner similar to Example 84.

TABLE 21

| | | Physical |
|---|---|---|
| Example | Chemical structure | constant, etc. |
| 85 | 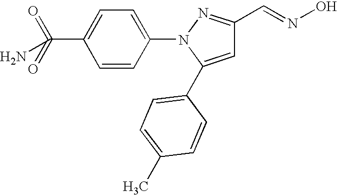 | MS: 357 [M + H]⁺, APCI (MeOH) |

### Example 86

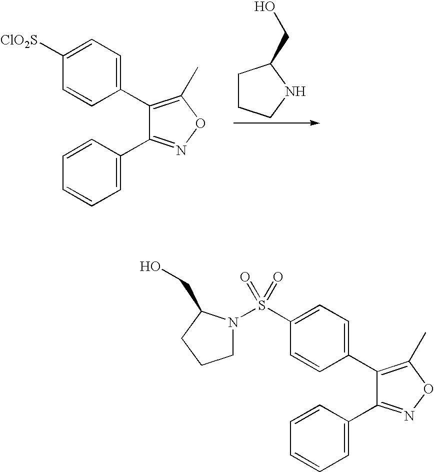

4-(5-Methyl-3-phenylisoxazol-4-yl)benzenesulfonyl chloride (200 mg, 0.60 mmol) was dissolved in THF (3 ml) and the solution was cooled to −78° C. After S-(−)-prolinol (182 mg, 1.80 mmol) was added to the solution, the mixture was gradually warmed to room temperature and stirred at room temperature for 6 hours. Ethyl acetate (8 ml) was added thereto, and the mixture was washed with water (3 ml) and subsequently with brine (2 ml), and concentrated under reduced pressure. The residue was purified by silica gel column chromatography (hexane:ethyl acetate=9:1->1:1) to give ((2S)-1-{[4-(5-methyl-3-phenylisoxazol-4-yl)-phenyl]sulfonyl}pyrrolidin-2-yl)methanol (232 mg, 97%) as a liquid.

MS: 399 [M+H]⁺, APCI (MeOH)

### Examples 87-108

The following compounds were prepared by carrying out a reaction and a treatment in a manner similar to Example 86.

TABLE 22

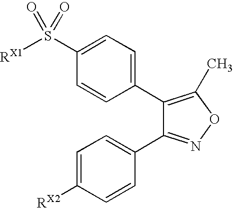

| | | | Physical |
|---|---|---|---|
| Example | R^{x1} | R^{x2} | constant, etc. |
| 87 | 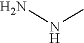 | H— | MS: 330 [M + H]⁺, APCI (MeOH) |
| 88 | 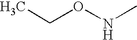 | H— | MS: 359 [M + H]⁺, APCI (MeOH) |
| 89 | 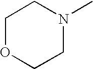 | H— | MS: 385 [M + H]⁺, APCI (MeOH) |
| 90 | 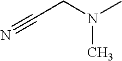 | H— | MS: 368 [M + H]⁺, APCI (MeOH) |
| 91 | 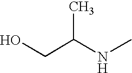 | H— | MS: 373 [M + H]⁺, APCI (MeOH) |
| 92 | 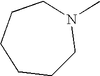 | H— | MS: 397 [M + H]⁺, APCI (MeOH) |
| 93 | 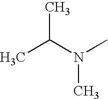 | H— | MS: 371 [M + H]⁺, APCI (MeOH) |
| 94 | 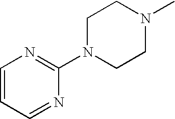 | H— | MS: 462 [M + H]⁺, APCI (MeOH) |
| 95 | 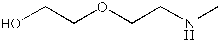 | H— | MS: 403 [M + H]⁺, APCI (MeOH) |
| 96 | 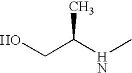 | H— | MS: 373 [M + H]⁺, APCI (MeOH) |
| 97 | 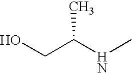 | H— | MS: 373 [M + H]⁺, APCI (MeOH) |
| 98 | 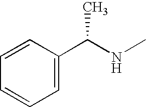 | H— | MS: 419 [M + H]⁺, ESI |
| 99 | 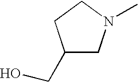 | H— | MS: 399 [M + H]⁺, ESI |
| 100 | 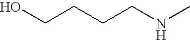 | H— | MS: 387 [M + H]⁺, ESI |
| 101 | 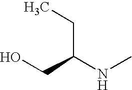 | H— | MS: 387 [M + H]⁺, ESI |
| 102 | 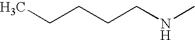 | H— | MS: 385 [M + H]⁺, ESI |
| 103 | 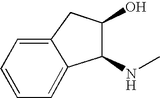 | H— | MS: 447 [M + H]⁺, ESI |
| 104 | 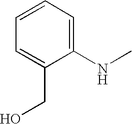 | H— | MS: 421 [M + H]⁺, ESI |
| 105 | 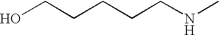 | H— | MS: 401 [M + H]⁺, ESI |
| 106 | 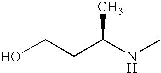 | H— | MS: 387 [M + H]⁺, APCI (MeOH) |
| 107 | 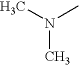 | H— | MS: 343 [M + H]⁺, APCI (MeOH) |
| 108 | 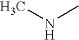 | Br— | MS: 407/409 [M + H]⁺, APCI (MeOH) |

### Example 109

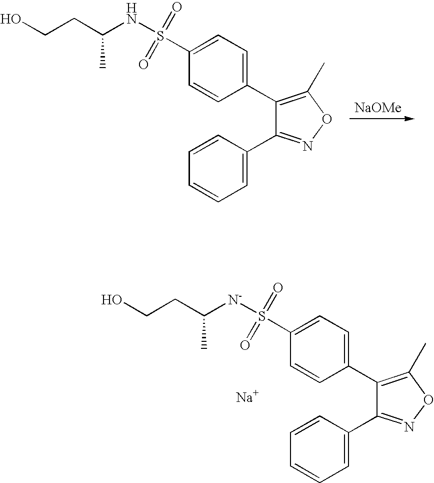

N-[(1R)-3-Hydroxy-1-methylpropyl]-4-(5-methyl-3-phenylisoxazol -4-yl)benzenesulfonamide (100 mg, 0.26 mmol) was dissolved in methanol (3 ml) and sodium methylate (0.5M methanol solution, 0.51 ml, 0.255 mmol) was added thereto at room temperature. After the mixture was stirred for 10 minutes, the reaction mixture was concentrated under reduced pressure. Acetone was added to the residue and the mixture was stirred, and then, the precipitate was collected by filtration to give N-[(1R)-3-hydroxy-1-methylpropyl]-4-(5-methyl-3-phenylisoxazol-4-yl)benzenesulfonamide-sodium salt (96 mg, 98%) as a solid.

MS: 385 [M−Na]⁻, ESI (MeOH).

### Examples 110-113

The following compounds were prepared by carrying out a reaction and a treatment in a manner similar to Example 109.

TABLE 23

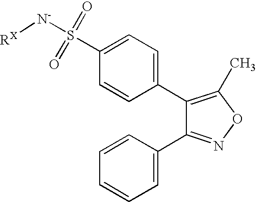

| | | Physical constant, |
|---|---|---|
| Example | R^{x} | etc. |
| 110 | 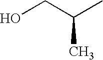 | MS: 371 [M − Na]⁻, ESI (MeOH) |
| 111 | 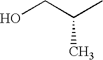 | MS: 371 [M − Na]⁻, ESI (MeOH) |
| 112 | 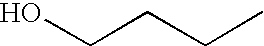 | MS: 373 [M − Na + 2H]⁺, APCI (MeOH) |

### Example 113

The following compound was prepared by carrying out a reaction and a treatment in a manner similar Example 70.

TABLE 24

| | | Physical |
|---|---|---|
| Example | Chemical structure | constant, etc. |
| 113 | 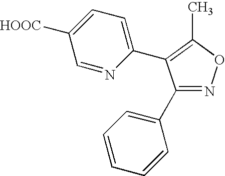 | MS: 279 [M − H]⁻, ESI |

### Example 114

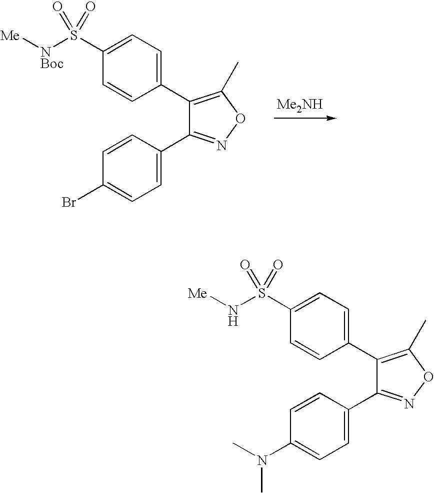

A solution of dimethylamine in THF (2M, 2.9 ml, 5.80 mmol) was added to a suspension of tert-butyl({4-[3-(4-bromophenyl) -5-methylisoxazol-4-yl]phenyl}sulfonyl)methyl carbamate (450 mg, 1.14 mmol), tris(dibenzilideneacetone) dipalladium (110 mg, 0.12 mmol), 2-dicyclohexylphosphino -2′-(N,N-dimethylamino)biphenyl (90 mg, 0.23 mmol) and sodium tert-butoxide (220 mg, 2.29 mmol) in toluene (15 ml) at room temperature. The mixture was heated to 80° C. in the sealed tube, and stirred for 20 hours. The suspension was poured into ethyl acetate/water. The organic layer was separated, washed with brine, dried over sodium sulfate, and concentrated under reduced pressure. The residue was purified by silica gel column chromatography (hexane:ethyl acetate=95:5->50:50) to give 4-{3-[4-(dimethylamino)phenyl]-5-methylisoxazol-4-yl}-N-methylbenzenesulfonamide (154 mg, 47%) as a solid.

MS: 372 [M+H]⁺, APCI (MeOH)

### Example 115

The following compound was prepared by carrying out a reaction and a treatment in a manner similar to Example 114.

TABLE 25

| | | Physical |
|---|---|---|
| Example | Chemical structure | constant, etc. |
| 115 | 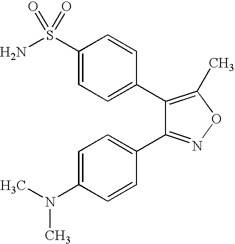 | MS: 358 [M + H]⁺, APCI (MeOH) |

### Example 116

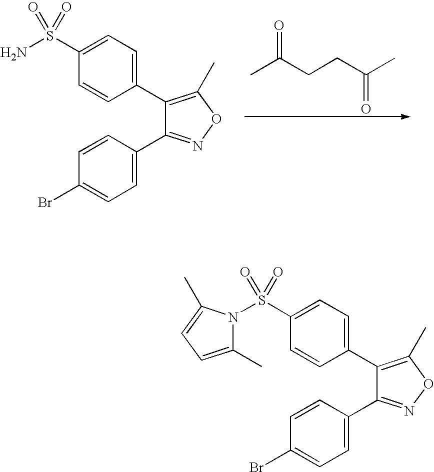

p-Toluenesulfonic acid monohydrate (0.18 g, 0.9 mmol) was added to a suspension of 4-[3-(4-bromophenyl)-5-methylisoxazol-4-yl]benzenesulfonamide (3.70 g, 9.4 mmol) and acetonylacetone (4.4 ml, 37.5 mmol) in toluene (100 ml) at room temperature. A reflux condenser equipped with Dean-Stark water separator was attached and the mixture was refluxed under heating for 15 hours. After allowing the mixture to cool, ethyl acetate (100 ml) was added to the reaction mixture. The mixture was washed with a saturated aqueous sodium bicarbonate solution and brine, dried over sodium sulfate, and concentrated under reduced pressure. The residue wag purified by silica gel column chromatography (hexane:ethyl acetate=2:1) to give 3-(4-bromophenyl) -4-{4-[(2,5-dimethyl-1H-pyrrol-1-yl)sulfonyl]-phenyl}-5-methylisoxazole (3.11 g, 70%) as a solid.

MS: 471/473 [M+H]⁺, APCI (MeOH)

### Example 117

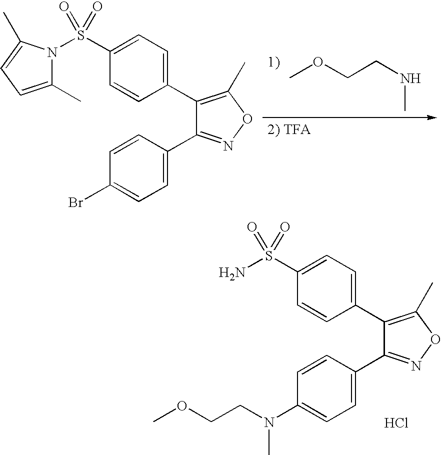

N-(2-Methoxyethyl)methylamine (60 mg, 0.67 mmol) was added to a suspension of 3-(4-bromophenyl)-4-{4-[(2,5-dimethyl-1H-pyrrol-1-yl)sufonyl]phenyl}-5-methylisoxazole (200 mg, 0.42 mmol), tris(dibenzylideneacetone) dipalladium (40 mg, 0.04 mmol), 2-dicyclohexylphosphino-2′-(N,N-dimethylamino)biphenyl (3-5 mg, 0.09 mmol) and cesium carbonate (280 mg, 0.86 mmol) in 1,4-dioxane (4 ml) and tert-butyl alcohol (2 ml) at room temperature, and the mixture was heated to 100° C. under microwave irradiation, and stirred for 1.5 hours. After the suspension was poured into ethyl acetate/water, the organic layer was washed with brine, dried over sodium sulfate, and concentrated under reduced pressure. The residue was purified by silica gel column chromatography (hexane:ethyl acetate=95:5->65:35) to give a solid. The solid was dissolved in trifluoroacetic acid (3 ml) and water (1 ml) and the mixture was heated to 60° C. and stirred for 6 hours. After the reaction mixture was allowed to cool, it was poured into a saturated aqueous sodium bicarbonate solution (25 ml), and the mixture was extracted with ethyl acetate (3×10 ml). The extract was washed with brine, dried over sodium sulfate, and concentrated under reduced pressure. The residue was purified by silica gel column chromatography (hexane:ethyl acetate=90:10->30:70) to give a liquid. To a solution of the liquid in methanol (0.5 ml) was added a 4N-hydrochloric acid-dioxane solution (2.0 ml) at room temperature and the mixture was stirred for 20 minutes. The reaction mixture was concentrated to give 4-(3-{4-[(2-methoxyethyl)(methyl)amino]phenyl}-5-methylisoxazol-4-yl)benzenesulfonamide hydrochloride (92 mg, 54%) as a solid.

MS: 402 [M+H]⁺, APCI (MeOH)

### Example 118 and 119

The following compounds were prepared by carrying out a reaction and a treatment in a manner similar to Example 117.

TABLE 26

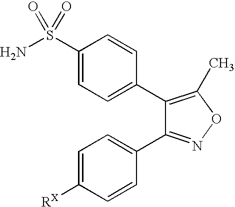

| | | Physical |
|---|---|---|
| Example | R^{x} | constant, etc. |
| 118 | 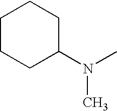 | MS: 426 [M + H]⁺, APCI (MeOH) |
| 119 | 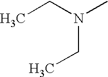 | MS: 386 [M + H]⁺, APCI (MeOH) |

### Example 120

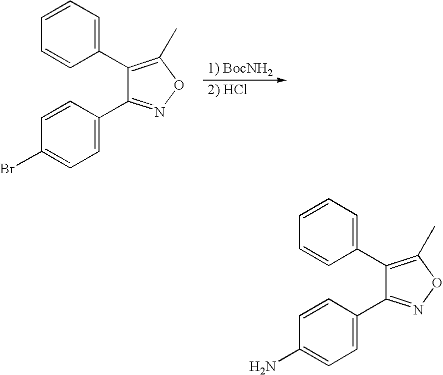

Sodium phenoxide (115 mg, 0.99 mmol) was added to a suspension of 3-(4-bromophenyl)-5-methyl-4-phenylisoxazole (200 mg, 0.64 mmol), tris(dibenzylideneacetone) dipalladium (60 mg, 0.07 mmol), 2-dicyclohexylphosphino-2′-(N,N-dimethylamino)biphenyl (50 mg, 0.13 mmol) and tert-butylcarbamate (115 mg, 0.98 mmol) in toluene (5 ml) at room temperature and the mixture was heated to 100° C. under microwave irradiation, and stirred for an hour. After the suspension was poured into ethyl acetate/water and the organic layer was washed with brine, it was dried over sodium sulfate, and concentrated under reduced pressure. The residue was purified by silica gel column chromatography (hexane:ethyl acetate=95:5->75:25) to give a solid. The solid was dissolved in a 4N hydrochloric acid-dioxane solution (5 ml) and the mixture was stirred at room temperature for 16 hours. The reaction mixture was poured into a saturated aqueous sodium bicarbonate solution (50 ml) and the mixture was extracted with ethyl acetate (3×10 ml). The extract was washed with brine, dried over sodium sulfate, and concentrated under reduced pressure. The residue was purified by silica gel column chromatography (hexane:ethyl acetate=95:5->50:50) to give [4-(5-methyl-4-phenylisoxazol-3-yl)phenyl]amine (116 mg, 73%) as a solid.

MS: 251 [M+H]⁺, APCI (MeOH)

### Example 121

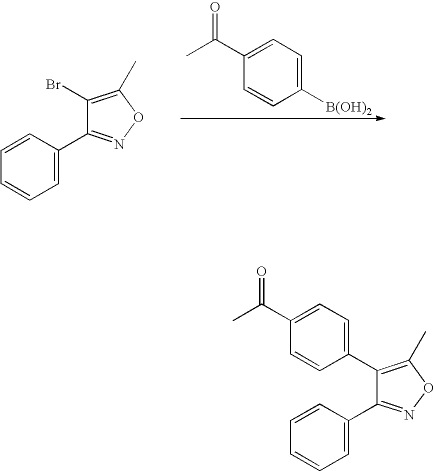

An aqueous sodium carbonate solution (2M, 1.3 ml, 2.60 mmol) was added to a suspension of 4-bromo-5-methyl-3-phenylisoxazole (200 mg, 0.84 mmol), 4-acetylphenylboric acid (210 mg, 1.28 mmol) and dichlorobis(triphenylphosphine)palladium (60 mg, 0.09 mmol) in DME (5 ml) at room temperature and the mixture was heated to 100° C. under microwave irradiation, and stirred for 2.5 hours. After the suspension was poured into ethyl acetate/water and the organic layer was washed with brine, it was dried over sodium sulfate and concentrated under reduced pressure. The residue was purified by silica gel column chromatography (hexane:ethyl acetate=98:2->65:35) to give 1-[4-(5-methyl-3-phenylisoxazol-4-yl)phenyl]ethanone (189 mg, 81%) as a liquid.

MS: 278 [M+H]⁺, APCI (MeOH)

### Examples 122-134

The following compounds were prepared by carrying out a reaction and a treatment in a manner similar to Example 121.

TABLE 27

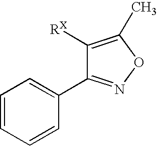

| | | Physical |
|---|---|---|
| Example | R^{x} | constant, etc. |
| 122 | 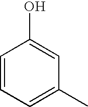 | MS: 252 [M + H]⁺, APCI (MeOH) |
| 123 | 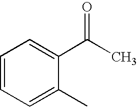 | MS: 278 [M + H]⁺, APCI (MeOH) |
| 124 | 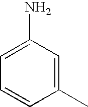 | MS: 251 [M + H]⁺, APCI (MeOH) |
| 125 | 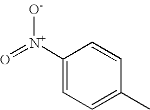 | MS: 281 [M + H]⁺, APCI (MeOH) |
| 126 | 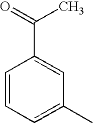 | MS: 278 [M + H]⁺, APCI (MeOH) |
| 127 | 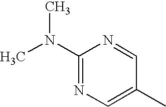 | MS: 281 [M + H]⁺, APCI (MeOH) |
| 128 | 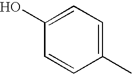 | MS: 252 [M + H]⁺, APCI (MeOH) |
| 129 | 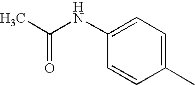 | MS: 293 [M + H]⁺, APCI (MeOH) |
| 130 | 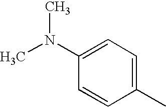 | MS: 279 [M + H]⁺, APCI (MeOH) |
| 131 | 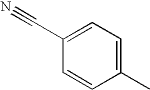 | MS: 261 [M + H]⁺, APCI (MeOH) |
| 132 | 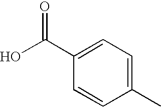 | MS: 280 [M + H]⁺, APCI (MeOH) |
| 133 | 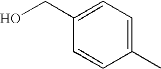 | MS: 266 [M + H]⁺, APCI (MeOH) |
| 134 | 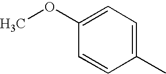 | MS: 266 [M + H]⁺, APCI (MeOH) |

### Example 135

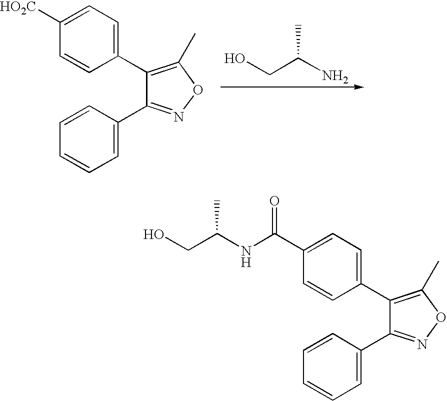

4-(5-Methyl-3-phenylisoxazol-4-yl)benzoic acid (100 mg, 0.36 mmol) and N-hydroxysuccinimide (72 mg, 0.38 mmol) were dissolved in DMF (3 ml), and 1-ethyl-3-(3′-dimethylaminopropyl)carbodiimide hydrochloride (45 mg, 0.39 mmol) was added thereto at 0° C. The reaction mixture was gradually warmed to room temperature and the mixture was stirred at room temperature overnight. Ethyl acetate (100 ml) was added to the mixture and the mixture was washed with a saturated aqueous sodium bicarbonate solution and water, dried over anhydrous magnesium sulfate and concentrated under reduced pressure. The residue was crystallized with hexane and collected by filtration. The crystal was dissolved in DMF (3 ml) and cooled to −78° C. After S-alaninol (30 mg, 0.4 mmol) was added to the solution, the reaction mixture was gradually warmed to room temperature, and the mixture was stirred at room temperature overnight. Ethyl acetate (20 ml) was added to the reaction mixture and the mixture was washed with a 10% aqueous citric acid solution and water and concentrated. The residue was purified by silica gel column chromatography (hexane:ethyl acetate=95:5->80:20) to give N-[(1S)-2-hydroxy-1-methylethyl]-4-(5-methyl-3-phenylisoxazol-4-yl)benzamide (105 mg, 87%) as a solid.

MS: 337 [M+H]⁺, APCI (MeOH)

### Examples 136-148

The following compounds were prepared by carrying out a reaction and a treatment in a manner similar to Example 135.

TABLE 28

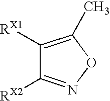

| | | | Physical |
|---|---|---|---|
| Example | R^{x1} | R^{x2} | constant, etc. |
| 136 | 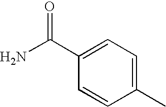 | 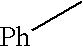 | MS: 279 [M + H]⁺, APCI (MeOH) |
| 137 | 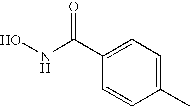 | 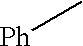 | MS: 295 [M + H]⁺, APCI (MeOH) |
| 138 | 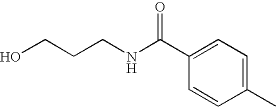 | 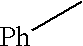 | MS: 337 [M + H]⁺, APCI (MeOH) |
| 139 | 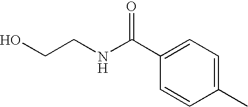 | 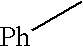 | MS: 323 [M + H]⁺, APCI (MeOH) |
| 140 | 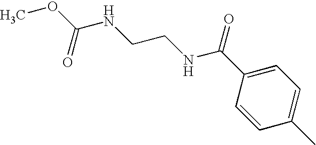 | 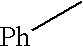 | MS: 380 [M + H]⁺, ESI |
| 141 | 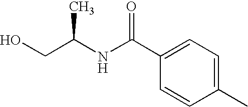 | 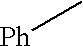 | MS: 337 [M + H]⁺, APCI (MeOH) |
| 142 | 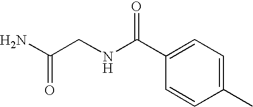 | 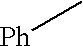 | MS: 336 [M + H]⁺, APCI (MeOH) |
| 143 | 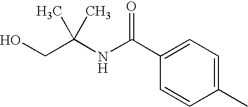 | 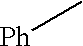 | MS: 351 [M + H]⁺, APCI (MeOH) |
| 144 | 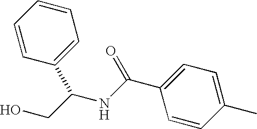 | 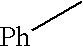 | MS: 399 [M + H]⁺, APCI (MeOH) |
| 145 | 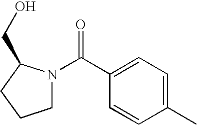 | 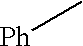 | MS: 363 [M + H]⁺, APCI (MeOH) |
| 146 | 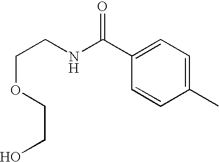 | 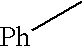 | MS: 367 [M + H]⁺, APCI (MeOH) |
| 147 | 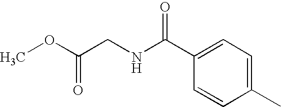 | 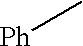 | MS: 351 [M + H]⁺, APCI (MeOH) |
| 148 | 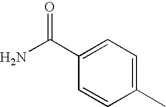 | 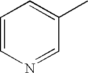 | MS: 280 [M + H]⁺, APCI (MeOH) |

### Example 149

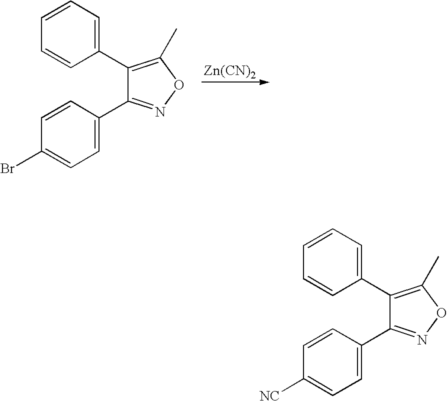

A suspension of 3-(4-bromophenyl)-5-methyl-4-phenylisoxazole (5.00 g, 15.9 mmol), zinc cyanide (1.88 g, 16.0 mmol) and tetrakis(triphenylphosphine) palladium (1.85 g, 1.60 mmol) in DMF (80 ml) was heated to 175° C. under microwave irradiation and stirred for 5 minutes. After the suspension was poured into ethyl acetate/water and the organic layer was washed with brine, it was dried over sodium sulfate and concentrated under reduced pressure. The residue was purified by silica gel column chromatography (hexane:ethyl acetate=6:1) to give 4-(5-methyl-4-phenylisoxazol -3-yl)benzonitrile (2.95 g, 71%) as powders.

MS: 261 [M+H]⁺, APCI (MeOH)

### Example 150

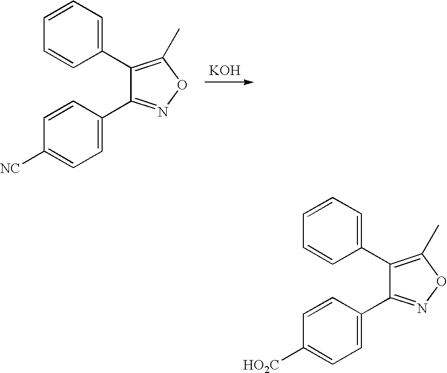

A suspension of 4-(5-methyl-4-phenylisoxazol-3-yl)benzonitrile (2.00 g, 7.7 mmol) and potassium hydroxide powder (2.40 g, 42.8 mmol) in 1-propanol (50 ml) was refluxed under heating for 14 hours. After cooling the reaction mixture, it was concentrated under reduced pressure. After 1N hydrochloric acid was added to the residue, the mixture was extracted with ethyl acetate. The organic layer was washed with brine, dried over sodium sulfate, and concentrated under reduced pressure. The residue was purified by silica gel column chromatography (chloroform:methanol=10:1) to give 4-(5-methyl-4-phenylisoxazol-3-yl)benzoic acid (2.01 g, 94%) as powders.

MS: 278 [M−H]⁻, ESI (MeOH)

### Example 151

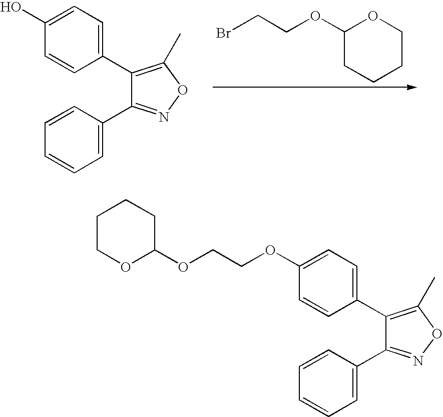

4-(5-Methyl-3-phenylisoxazol-4-yl)phenol (150 mg, 0.60 mmol) was dissolved in DMF (3 ml) and 60% sodium hydride (27 mg, 0.68 mmol) was added thereto at room temperature. After 10 minutes, 2-(2-bromoethoxy)tetrahydro-2H-pyrane (137 mg, 0.66 mmol) was added to the mixture at room temperature and the mixture was stirred overnight. To the reaction mixture was added ethyl acetate (200 ml) and the mixture was washed with water, and then dried over sodium sulfate. After the solvent was removed under reduced pressure, the residue was purified by silica gel column chromatography (hexane:ethyl acetate=90:10->70:30) to give 5-methyl-3-phenyl-4-{4-[2-(tetrahydro-2H-pyran-2-yloxy)ethoxy]phenyl}isoxazole (141 mg, 62%) as an oil.

MS: 380 [M+H]⁺, APCI (MeOH)

### Example 152

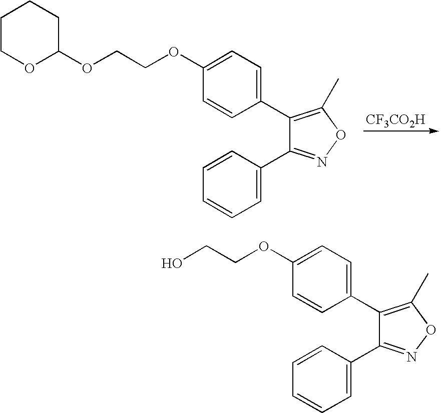

5-Methyl-3-phenyl-4-{4-[2-(tetrahydro-2H-pyran-2-yloxy)-ethoxy]phenyl}isoxazole (140 mg, 0.37 mmol) was dissolved in trifluoroacetic acid (4 ml) and the mixture was stirred at room temperature for 6 hours. After the reaction mixture was concentrated under reduced pressure, the residue was purified by silica gel column chromatography (hexane:ethyl acetate=90:10->0:100) to give 2-[4-(5-methyl-3-phenylisoxazol-4-yl)phenoxy]ethanol (52 mg, 47%) as powders.

MS: 296 [M+H]⁺, APCI (MeOH)

### Example 153

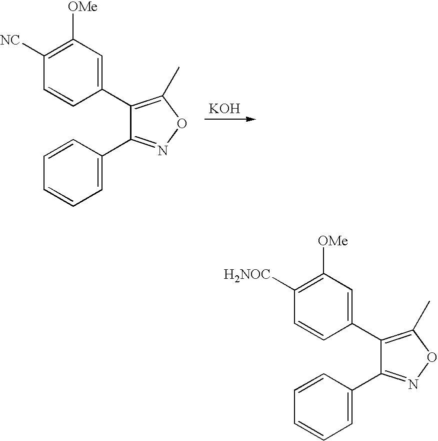

Potassium hydroxide powder (197 mg, 3.50 mmol) was added to a solution of 2-methoxy-4-(5-methyl-3-phenylisoxazol-4-yl)benzonitrile (109 mg, 0.377 mmol) in tert-butanol (4.0 ml) and the mixture was refluxed under heating for 5 hours. After cooling the reaction mixture, brine was added thereto and the mixture was extracted with ethyl acetate. The organic layer was dried over sodium sulfate and concentrated under reduced pressure. The residue was purified by silica gel column chromatography (hexane:ethyl acetate=1:2) to give 2-methoxy-4-(5-methyl-3-phenylisoxazol-4-yl)benzamide (273 mg, 73%) as a solid.

MS: 309 [M+H]⁺, APCI (MeOH)

### Example 154

The following compound was prepared by carrying out a reaction and a treatment in a manner similar to Example 153.

TABLE 29

| | | Physical |
|---|---|---|
| Example | Chemical structure | constant, etc. |
| 154 | 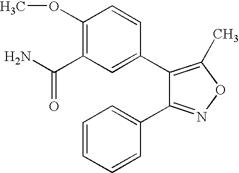 | MS: 309 [M + H]⁺, APCI (MeOH) |

### Example 155

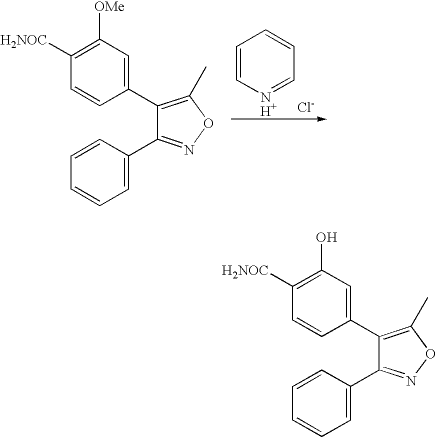

Pyridinium chloride (270 mg, 2.34 mmol) was added to 2-methoxy-4-(5-methyl-3-phenylisoxazol-4-yl)benzamide (45 mg, 0.146 mmol) and the mixture was heated at 190° C. for 2 hours. Water was added to the reaction mixture and the mixture was extracted with ethyl acetate. The organic layer was washed with brine, dried over sodium sulfate and concentrated under reduced pressure. The residue was purified by silica gel column chromatography (hexane:ethyl acetate=1:1) to give 2-hydroxy-4-(5-methyl-3-phenylisoxazol -4-yl)benzamide (34.9 mg, 81%) as a solid.

MS: 293[M−H]⁻, ESI (MeOH)

### Example 156

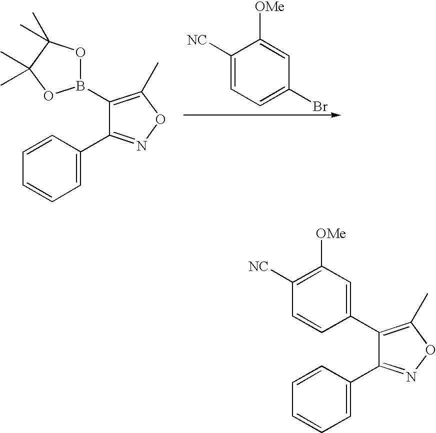

A suspension of 5-methyl-3-phenyl-4-(4,4,5,5-tetramethyl -1,3,2-dioxabororan-2-yl)isoxazole (605 mg, 2.12 mmol), 4-bromo-2-methoxybenzonitrile (300 mg, 1.415 mmol), palladium acetate (31.7 mg, 0.142 mmol), 2-dicyclohexylphosphino-2′-(N,N-dimethylamino)biphenyl (111 mg, 0.283 mmol) and potassium phosphate (901 mg, 4.245 mmol) in toluene (7.0 ml) was stirred for 24 hours with heating. After the suspension was poured into ethyl acetate/water and the organic layer was washed with brine, dried over sodium sulfate and concentrated under reduced pressure. The residue was purified by silica gel column chromatography (hexane:ethyl acetate=3:1) to give 2-methoxy-4-(5-methyl-3-phenylisoxazol-4-yl)benzonitrile (188 mg, 46%) as a solid.

MS: 291 [M+H]⁺, APCI (MeOH)

### Example 157

The following compound was prepared by carrying out a reaction and a treatment in a manner similar to Example 156.

TABLE 30

| | | Physical |
|---|---|---|
| Example | Chemical structure | constant, etc. |
| 157 | 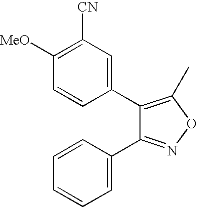 | MS: 291 [M + H]⁺, APCI (MeOH) |

### Example 158

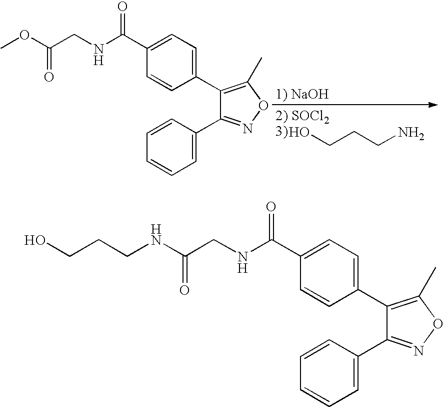

Methyl-N-[4-(5-methyl-3-phenylisoxazol-4-yl)benzoyl]glycinate (138 mg, 0.39 mmol) was dissolved in methanol (1 ml) and a 1N aqueous sodium hydroxide solution (945 μl) was added thereto, and the mixture was stirred at room temperature for 2 hours. The reaction-mixture was concentrated under reduced pressure and a 10% aqueous hydrochloric acid solution-ethyl acetate was added thereto. The organic layer was separated, washed with brine, dried over magnesium sulfate and concentrated under reduced pressure to give a crude product of N-[4-(5-methyl-3-phenylisoxazol-4-yl)benzoyl]glycine. Without isolating the obtained crude product, thionyl chloride was added thereto, and the mixture was refluxed for 2 hours. The reaction mixture was concentrated and diluted with dichloromethane (2 ml). It was added dropwise to a solution of 3-amino-1-propanol (59 mg, 0.79 mmol) and triethylamine (80 mg, 0.79 mmol) in dichloromethane at −78° C., and the mixture was further stirred at room temperature overnight. The reaction mixture was concentrated and the residue was purified to give N-[(3-hydroxypropylamino)carbonylmethyl]-4-(5-methyl-3-phenylisoxazol-4-yl)benzamide (56 mg, 36%) as powders.

MS: 394 [M+H]⁺, APCI (MeOH)

### Example 159

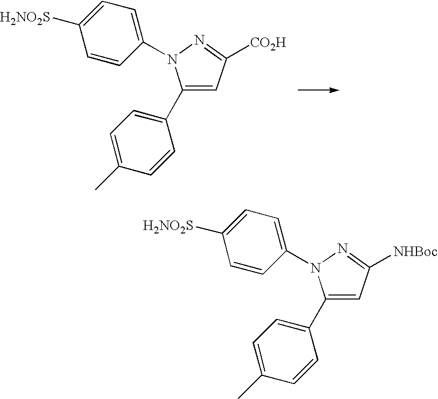

A solution of 1-[4-(aminosulfonyl)phenyl]-5-(4-methylphenyl)-1H-pyrazol-3-carboxylic acid (2.14 g, 6 mmol), diphenylphosphonylazide (1.55 ml, 7.2 mmol) and triethylamine (1.00 ml, 7.2 mmol) in tert-butanol (30 ml) and 1,4-dioxane (30 ml) was refluxed under heating for 16 hours. After cooling the reaction mixture with ice, ethyl acetate and water were added. The organic layer was separated, washed with brine, dried over magnesium sulfate and concentrated under reduced pressure. The residue was purified by silica gel column chromatography (chloroform:methanol=50:1->20:1) to give 1-(4-aminosulfonylphenyl) -3-(tert-butoxycarbonylamino)-5-(4-methylphenyl) -1H-pyrazole (569 mg, 22%) as a solid.

MS: 429 [M+H]⁺, APCI (10 mM-AcONH₄/MeOH)

### Example 160

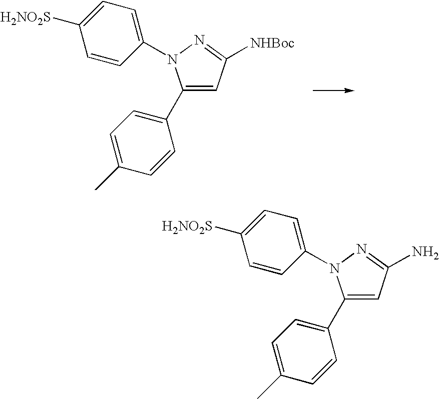

Trifluoroacetic acid (2 ml) was added to a solution of 1-(4-aminosulfonylphenyl)-3-(tert-butoxycarbonylamino)-5-(4-methylphenyl)-1H-pyrazole (510 mg, 1.19 mmol) in chloroform (5 ml) and the mixture was stirred. Ethyl acetate and a saturated aqueous sodium bicarbonate solution were added to the reaction mixture. The organic layer was separated, washed with brine, dried over sodium sulfate, and concentrated under reduced pressure. Diethyl ether was added to the residue and the precipitated solid was collected by filtration to give 3-amino-1-(4-aminosulfonylphenyl) -5-(4-methylphenyl)-1H-pyrazole (295 mg, 75%) as a solid.

MS: 329 [M+H]⁺, APCI (MeOH)

### Example 161

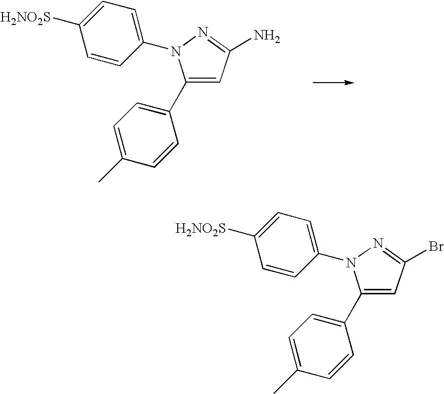

Water (2 ml) and a 48% aqueous HBr solution (1 ml) were added to 3-amino-1-(4-aminosulfonylphenyl)-5-(4-methylphenyl)-1H-pyrazole (66 mg, 0.2 mmol). To the mixture were added an aqueous sodium nitrite (17 mg, 0.24 mmol) solution (0.5 ml) and acetonitrile (2 ml) under ice-cooling and the mixture was stirred for 10 minutes. To the obtained reaction mixture was added a solution of CuBr (43 mg, 0.3 mmol) in a 48% aqueous HBr solution (0.5 ml) at room temperature, and the mixture was stirred at 80° C. for 30 minutes. Ethyl acetate and water were added to the reaction mixture. The organic layer was separated, washed with brine, dried over sodium sulfate, and concentrated under reduced pressure. The residue was purified by silica gel column chromatography (chloroform:methanol=50:1) to give 1-[4-(aminosulfonyl)phenyl]-3-bromo-5-(4-methylphenyl)-1H-pyrazole (33 mg, 39%).

MS: 392/394 [M+H]⁺, APCI (MeOH)

### Example 162

(1)

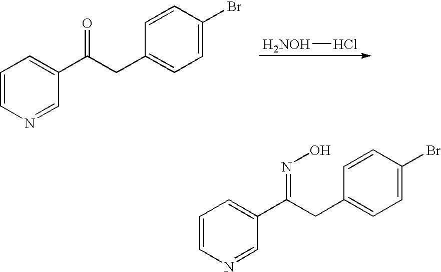

A suspension of 2-(4-bromophenyl)-1-pyridin-3-ylethanone (8.00 g, 27.5 mmol), hydroxylamine hydrochloride (2.00 g, 28.8 mmol) and sodium bicarbonate (2.45 g, 29.2 mmol) in ethanol (70 ml) and water (10 ml) was stirred for 3 hours at 60° C. After the solvent was removed under reduced pressure, ethyl acetate/water was added to the residue. The organic layer was washed with brine, dried over sodium sulfate, and concentrated under reduced pressure. The residue was purified by silica gel column chromatography (hexane:ethyl acetate=1:1) to give 2-(4-bromophenyl)-1-pyridin-3-ylethanone oxime (7.95 g, 94%) as powders.

MS: 291/293 [M+H]⁺, APCI (MeOH)

(2)

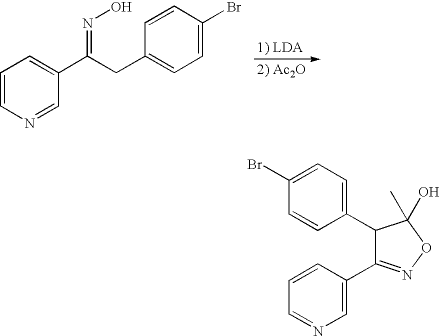

2-(4-Bromophenyl)-1-pyridin-3-ylethanone oxime (4.0 g, 13.7 mmol) was dissolved in THF (40 ml) and a 2M lithium diisopropylamide solution (heptane/THF/ethylbenzene solution) (15.1 ml, 30.2 mmol) was added dropwise thereto at −60° C.; After the addition, the reaction mixture was warmed to −30° C. and acetic anhydride (1.55 ml, 16.4 mmol) was added thereto in one portion. After the mixture was stirred at room temperature for an hour, the reaction mixture was poured into ethyl acetate/water. The organic layer was separated, washed with brine, dried over sodium sulfate and concentrated under reduced pressure. The residue was purified by silica gel column chromatography (hexane:ethyl acetate=1:1) to give 4-(4-bromophenyl)-5-methyl-3-pyridin-3-yl-4,5-dihydroisoxazol-5-ol (2.54 g, 56%) as powders.

MS: 333/335 [M+H]⁺, APCI (MeOH)

(3)

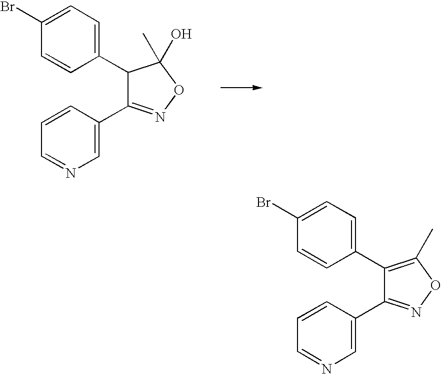

A suspension of 4-(4-bromophenyl)-5-methyl-3-pyridin-3-yl-4,5-dihydroisoxazol-5-ol (2.5 g, 7.6 mmol) and p-toluenesulfonic acid monohydrate (1.7 g, 9.1 mmol) in methanol (25 ml) was refluxed under heating for 24 hours. After cooling, the reaction mixture was concentrated under reduced pressure and ethyl acetate/a saturated aqueous sodium bicarbonate solution was added thereto. The organic layer was separated, washed with brine, dried over sodium sulfate and concentrated under reduced pressure. The residue was purified by silica gel column chromatography (hexane:ethyl acetate=3:1) to give 3-[4-(4-bromophenyl)-5-methylisoxazol-3-yl]pyridine (1.9 g, 78%) as a liquid.

MS: 315/317 [M+H]⁺, APCI (MeOH)

(4)

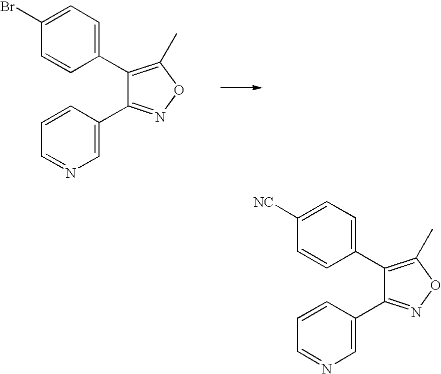

4-[5-Methyl-3-(3-pyridyl)isoxazol-4-yl]benzonitrile was prepared by reacting and treating the compound obtained in the above (3) in a manner similar to Example 149.

MS: 262 [M+H]⁺, APCI (MeOH)

(5)

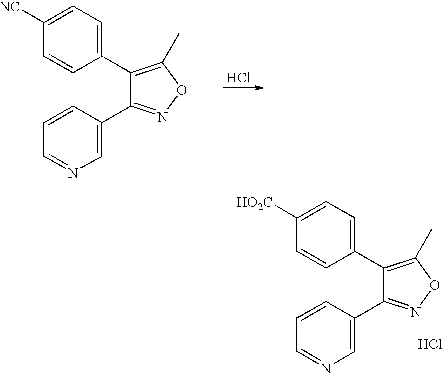

4-[5-Methyl-3-(3-pyridyl)isoxazol-4-yl]benzoic acid was prepared as a hydrochloride by reacting and treating the compound obtained in the above (4) in a manner similar to Example 149, and reacting and treating in a manner similar to Example 150, using 6N hydrochloric acid in place of potassium hydroxide.

MS: 279 [M−H]⁻, ESI (MeOH)

The following compounds were prepared by carrying out a reaction and a treatment in a manner similar to the above Examples or according to a known method generally employed.

TABLE 31

| | | Physical |
|---|---|---|
| Example | Chemical structure | constant, etc. |
| 163 | 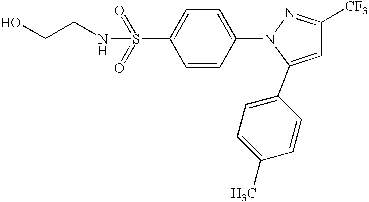 | MS: 426 [M + H]⁺, APCI (MeOH) |
| 164 | 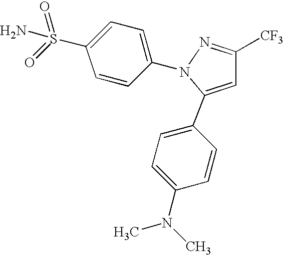 | MS: 411 [M + H]⁺, APCI (MeOH) |
| 165 | 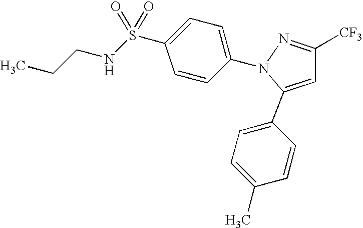 | MS: 424 [M + H]⁺, APCI (MeOH) |
| 166 | 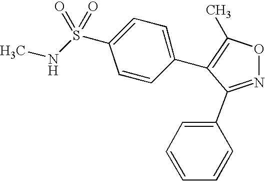 | MS: 327 [M − H]⁻ESI (MeOH) |
| 167 | 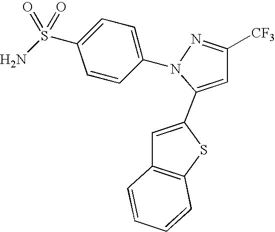 | MS: 424 [M + H]⁺, APCI (MeOH) |
| 168 | 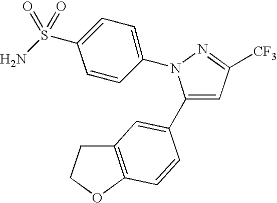 | MS: 410 [M + H]⁺, APCI (MeOH) |
| 169 | 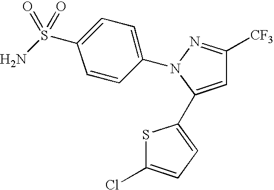 | 408/410 [M + H]⁺APCI (MeOH) |
| 170 | 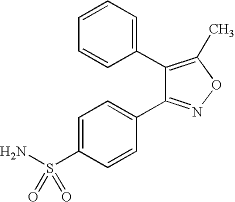 | MS: 315 [M + H]⁺, APCI (MeOH) |
| 171 | 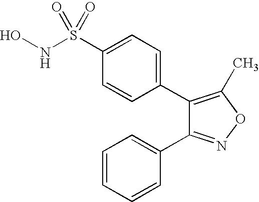 | MS: 331 [M + H]⁺, APCI (MeOH) |
| 172 | 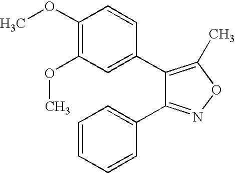 | MS: 296 [M + H]⁺, APCI (MeOH) |

### Example 173

The following compound was prepared by carrying out a reaction and a treatment in a manner similar to Example 72.

TABLE 32

| | | Physical |
|---|---|---|
| Example | Chemical structure | constant, etc. |
| 173 | 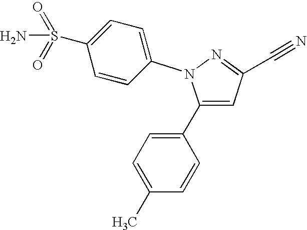 | MS: 337 [M − H]⁻, ESI (MeOH) |

### Example 174

Compound described in Journal of Medicinal Chemistry, vol 40, 1347-1365 (1997).

TABLE 33

| Example | Chemical structure |
|---|---|
| 174 | 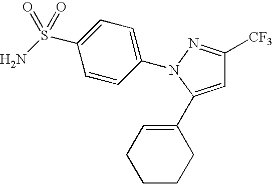 |

### Examples 175 and 176

The following compounds were prepared by carrying out a reaction and a treatment in a manner similar to Example 121 using 4-bromo-5-methyl-3-phenylisoxazole.

TABLE 34

| | | Physical |
|---|---|---|
| Example | Chemical structure | constant, etc. |
| 175 | 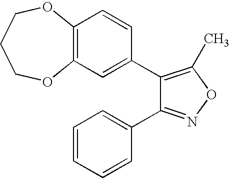 | MS: 308 [M + H]⁺, APCI (MeOH) |
| 176 | 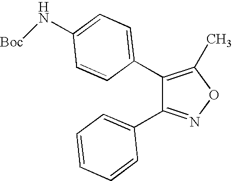 | MS: 351 [M + H]⁺, APCI (10 mM- AcONH₄/MeOH) |

### Example 177

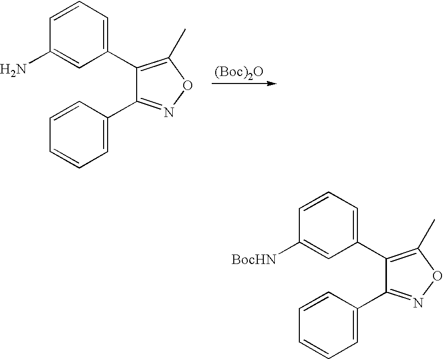

[3-(5-Methyl-3-phenylisoxazol-4-yl)phenyl]amine (500 mg, 2.00 mmol) was dissolved in 1,4-dioxane (5 ml) and di-tert-butyl dicarbonate was added thereto at room temperature, and the mixture was stirred at 90° C. for 6 hours. The reaction mixture was cooled to room temperature and a 10% aqueous citric acid solution (15 ml) was added thereto, and the mixture was extracted with ethyl acetate. The extract was washed with brine, dried over sodium sulfate and concentrated under reduced pressure. The residue was purified by silica gel column chromatography (hexane:ethyl acetate=5:1->3:1) to give t-butyl[3-(5-methyl-3-phenylisoxazol-4-yl)phenyl]carbamate (553 mg, 82%) as a solid.

MS: 3.51 [M+H]⁺, APCI (10 mM-AcONH₄/MeOH)

### Example 178

The following compound was prepared by reacting and treating the compound obtained in Example 177 in a manner similar to Example 151.

TABLE 35

| | | Physical |
|---|---|---|
| Example | Chemical structure | constant, etc. |
| 178 | 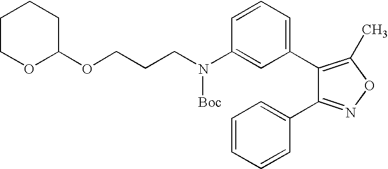 | MS: 493 [M + H]⁺, APCI (10 mM- AcONH₄/MeOH) |

### Examples 179 and 180

The following compounds were prepared by reacting and treating the compounds obtained in Examples 176 and 178 in a manner similar to Example 29.

TABLE 36

| Exam- | | Physical |
|---|---|---|
| ple | Chemical structure | constant, etc. |
| 179 | 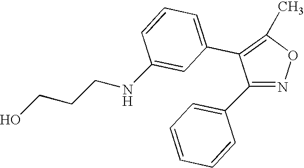 | MS: 309 [M + H]⁺, APCI (MeOH) |
| 180 | 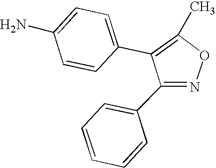 | MS: 287 [M + H]⁺, APCI (MeOH) |

### Examples 181-184

The following compounds were prepared by reacting and treating the compound obtained in Example 44 (1) in a manner similar to Example 44 (2).

TABLE 37

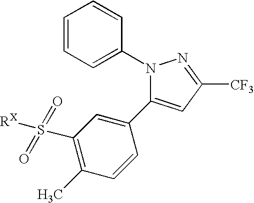

| | | Physical |
|---|---|---|
| Example | R^{x} | constant, etc. |
| 181 | 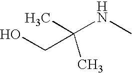 | MS: 454 [M + H]⁺, ESI |
| 182 | 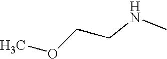 | MS: 440 [M + H]⁺, ESI |
| 183 | 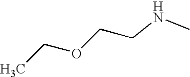 | MS: 454 [M + H]⁺, ESI |
| 184 | 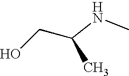 | MS: 440 [M + H]⁺, ESI |

### Examples 185-187

The following compounds were prepared by carrying out a reaction and a treatment in a manner similar to Example 156, using 5-methyl-3-phenyl-4-(4,4,5,5-tetramethyl-1,3,2-dioxabororan-2-yl)isoxazole.

TABLE 38

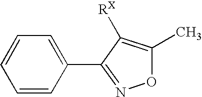

| | | Physical |
|---|---|---|
| Example | R^{x} | constant, etc. |
| 185 | 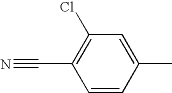 | MS: 295/297 [M + H]⁺, APCI (MeOH) |
| 186 | 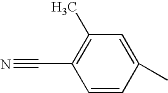 | MS: 275 [M + H]⁺, APCI (MeOH) |
| 187 | 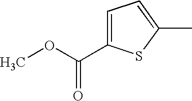 | MS: 300 [M + H]⁺, APCI (MeOH) |

### Examples 188 and 189

The following compounds were prepared by reacting and treating the compounds obtained in Example 156 and Example 185 in a manner similar to Example 150.

TABLE 39

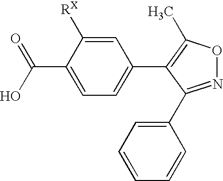

| Example | R^{x} | Physical constant, etc. |
|---|---|---|
| 188 | MeO— | MS: 308 [M − H]⁻, ESI |
| 189 | Cl— | MS: 312/314 [M − H]⁻, ESI |

### Examples 190 and 191

The following compounds were prepared by reacting and treating the compounds obtained in Examples 185 and 186 in a manner similar to Example 153.

TABLE 40

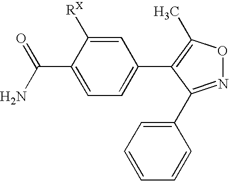

| Example | R^{x} | Physical constant, etc. |
|---|---|---|
| 190 | Cl— | MS: 313/315 [M + H]⁺, APCI (MeOH) |
| 191 | Me— | MS: 293 [M + H]⁺, APCI (MeOH) |

### Examples 192-222

The following compounds were prepared by carrying out a reaction and a treatment in a manner similar to Example 48.

TABLE 41

| | | Physical |
|---|---|---|
| Example | Chemical structure | constant, etc. |
| 192 | 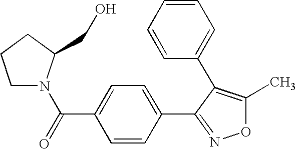 | MS: 363 [M + H]⁺, APCI (MeOH) |
| 193 | 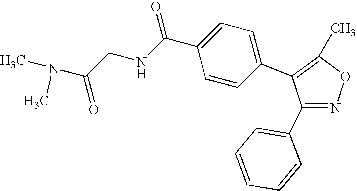 | MS: 364 [M + H]⁺, APCI (MeOH) |
| 194 | 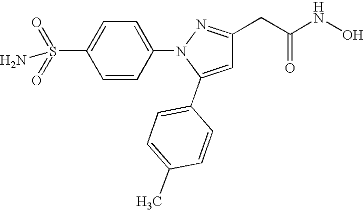 | MS: 387 [M + H]⁺, APCI (MeOH) |
| 195 | 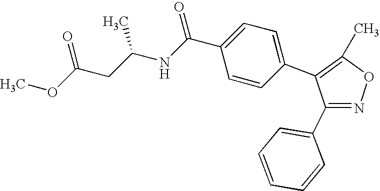 | MS: 379 [M + H]⁺, APCI (MeOH) |
| 196 | 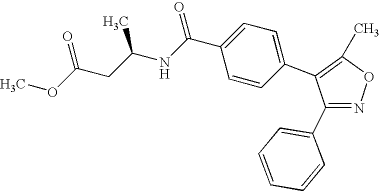 | MS: 379 [M + H]⁺, APCI (MeOH) |
| 197 | 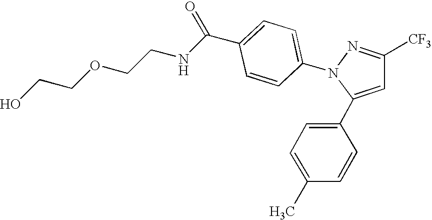 | MS: 434 [M + H]⁺, APCI (MeOH) |
| 198 | 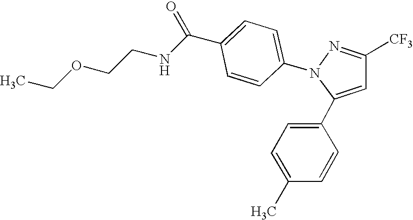 | MS: 418 [M + H]⁺, APCI (MeOH) |
| 199 | 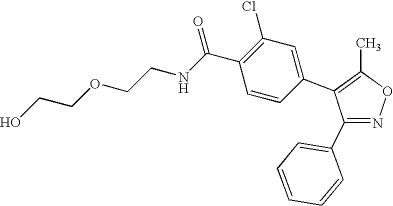 | MS: 401/403 [M + H]⁺, APCI (MeOH) |
| 200 | 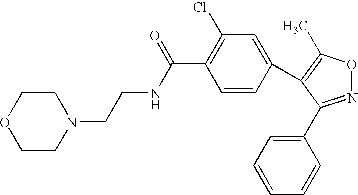 | MS: 426/428 [M + H]⁺, APCI (MeOH) |
| 201 | 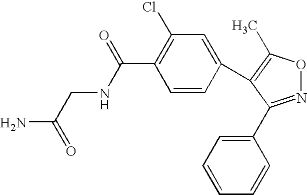 | MS: 370/372 [M + H]⁺, APCI (MeOH) |
| 202 | 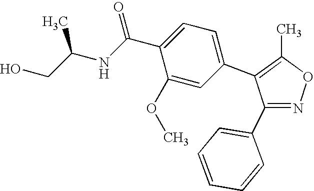 | MS: 367 [M + H]⁺, APCI (MeOH) |
| 203 | 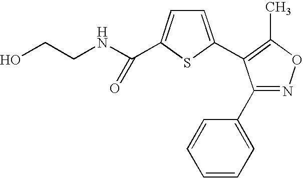 | MS: 329 [M + H]⁺, APCI (MeOH) |
| 204 | 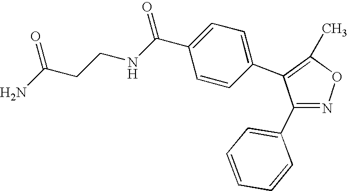 | MS: 350 [M + H]⁺, APCI (MeOH) |
| 205 | 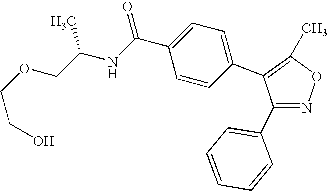 | MS: 381 [M + H]⁺, APCI (MeOH) |
| 206 | 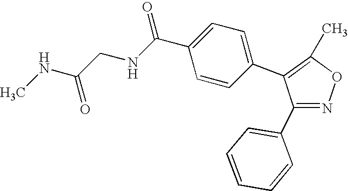 | MS: 350 [M + H]⁺, APCI (MeOH) |
| 207 | 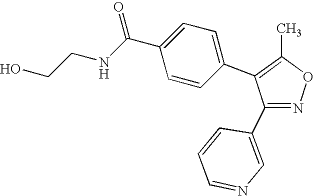 | MS: 324 [M + H]⁺, APCI (MeOH) |
| 208 | 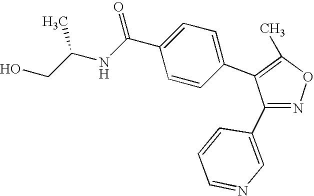 | MS: 338 [M + H]⁺, APCI (MeOH) |
| 209 | 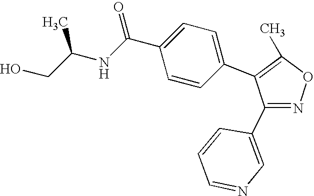 | MS: 338 [M + H]⁺, APCI (MeOH) |
| 210 | 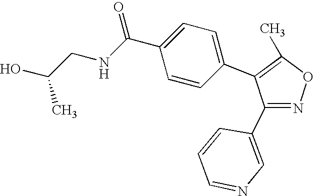 | MS: 338 [M + H]⁺, APCI (MeOH) |
| 211 | 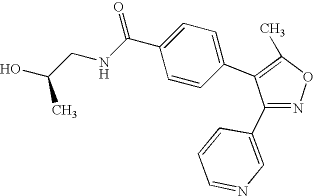 | MS: 338 [M + H]⁺, APCI (MeOH) |
| 212 | 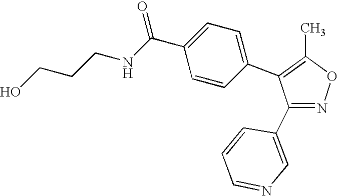 | MS: 338 [M + H]⁺, APCI (MeOH) |
| 213 | 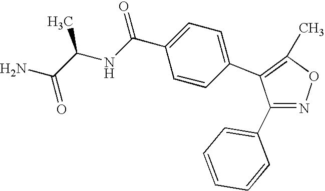 | MS: 350 [M + H]⁺, APCI (MeOH) |
| 214 | 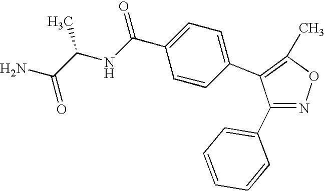 | MS: 350 [M + H]⁺, APCI (MeOH) |
| 215 | 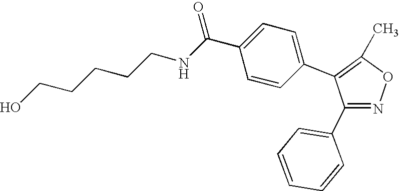 | MS: 365 [M + H]⁺, APCI (MeOH) |
| 216 | 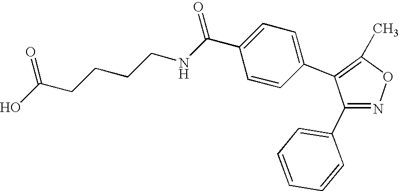 | MS: 377 [M − H]⁻, ESI |
| 217 | 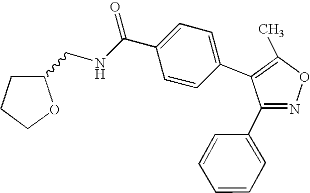 | MS: 363 [M + H]⁺, ESI |
| 218 | 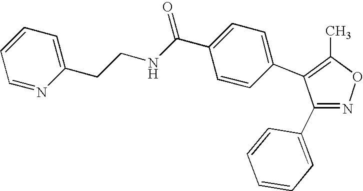 | MS: 384 [M + H]⁺, ESI |
| 219 | 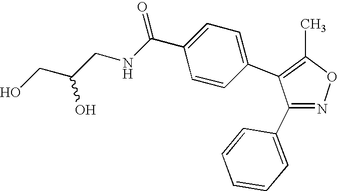 | MS: 353 [M + H]⁺, ESI |
| 220 | 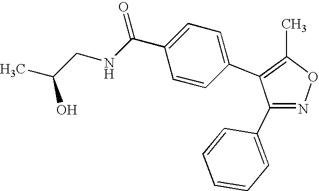 | MS: 337 [M + H]⁺, ESI |
| 221 | 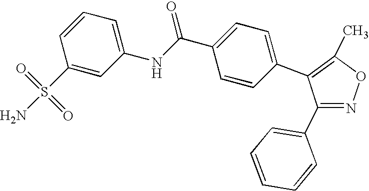 | MS: 434 [M + H]⁺, APCI (MeOH) |
| 222 | 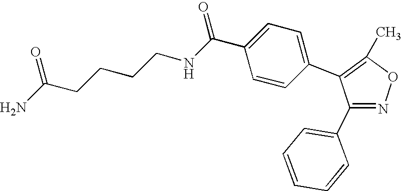 | MS: 378 [M + H]⁺, APCI (MeOH) |

### Examples 223 and 224

The following compounds were prepared by reacting and treating the compounds obtained in Example 187 and Example 194 in a manner similar to Example 70.

TABLE 42

| | | Physical |
|---|---|---|
| Example | Chemical structure | constant, etc. |
| 223 | 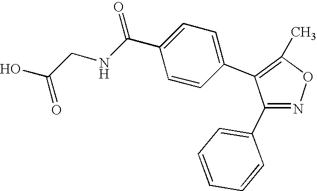 | MS: 335 [M − H]⁻, ESI |
| 224 | 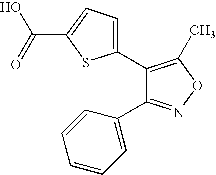 | MS: 284 [M − H]⁻, ESI |

### Example 225

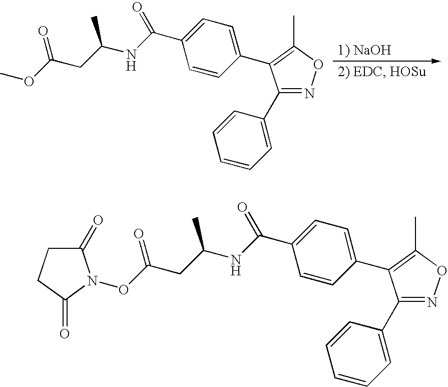

Methyl (3R)-3-{[(4-(5-methyl-3-phenylisoxazol-4-yl)-benzoyl)amino]butanoate (952 mg, 2.52 mmol) was dissolved in methanol (5 ml) and a 1N aqueous sodium hydroxide solution (3 ml, 3.0 mmol) was added thereto at 0° C., and the mixture was stirred at room temperature for 2 hours. After the reaction mixture was concentrated, water (60 ml) was added thereto and the mixture was washed with diethyl ether. The pH of the aqueous layer was adjusted to 3 with 10% hydrochloric acid and the mixture was extracted with ethyl acetate. The extract was washed with brine, dried over sodium sulfate, and concentrated under reduced pressure. DMF (3 ml) was added to the residue, and successively, N-hydroxysuccinimide (290 mg, 2.52 mmol) and 1-ethyl-3-(3′-dimethylaminopropyl)carbodiimide hydrochloride (482 mg, 2.52 mmol) were added thereto at 0° C. The mixture was gradually warmed to room temperature and stirred at room temperature overnight. Ethyl acetate (100 ml) was added thereto and the mixture was washed with a saturated aqueous sodium bicarbonate solution and water, dried over anhydrous magnesium sulfate, and concentrated. The residue was crystallized with hexane and collect by filtration to give N-{(1R)-3-[(2,5-dioxopyrrolidin-1-yl)oxy]-1-methyl-3-oxopropyl}-4-(5-methyl-3-phenylisoxazol-4-yl)benzamide (875 mg, 75%) as a solid.

MS: 462 [M+H]⁺, APCI (MeOH)

### Example 226

The following compound was prepared by carrying out a reaction and a treatment in a manner similar to Example 225.

TABLE 43

| | | Physical |
|---|---|---|
| Example | Chemical structure | constant, etc. |
| 226 | 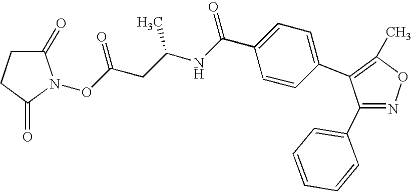 | MS: 462 [M + H]⁺, APCI (MeOH) |

### Example 227

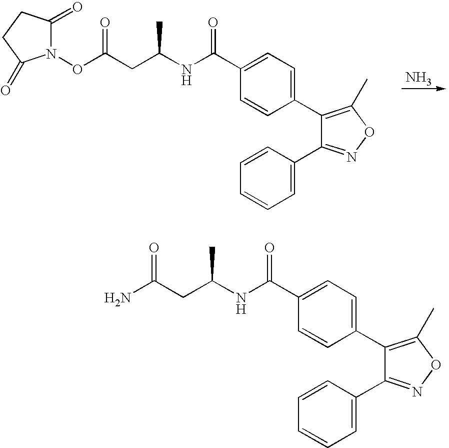

N-{(1R)-3-[(2,5-dioxopyrrolidin-1-yl)oxy]-1-methyl-3-oxopropyl}-4-(5-methyl-3-phenylisoxazol-4-yl)benzamide (100 mg, 0.22 mmol) was dissolved in THF (5 ml) and 30% aqueous ammonia (1 ml) was added thereto under ice-cooling. After the mixture was stirred at room temperature overnight, the reaction mixture was poured into ethyl acetate/water. The organic layer was washed with brine, dried over sodium sulfate and concentrated under reduced pressure. The residue was purified by silica gel column chromatography (hexane:ethyl acetate=75:25->0:100) to give N-[(1R)-3-amino-1-methyl-3-oxypropyl]-4-(5-methyl-3-phenylisoxazol-4-yl)berizamide (875 mg, 75%) as a solid.

MS: 364 [M+H]⁺, APCI (MeOH)

### Examples 228-230

The following compounds were prepared by carrying out a reaction and a treatment in a manner similar to Example 227.

TABLE 44

| | | Physical |
|---|---|---|
| Example | Chemical structure | constant, etc. |
| 228 | 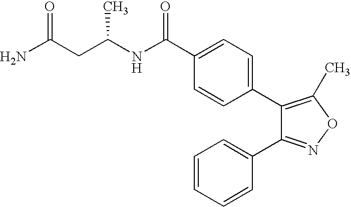 | MS: 364 [M + H]⁺, APCI (MeOH) |
| 229 | 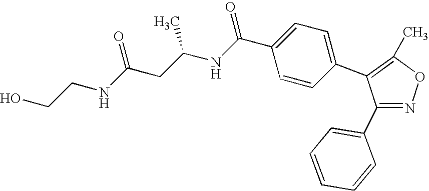 | MS: 408 [M + H]⁺, APCI (MeOH) |
| 230 | 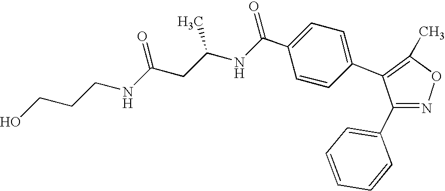 | MS: 422 [M + H]⁺, APCI (MeOH) |

### Example 231

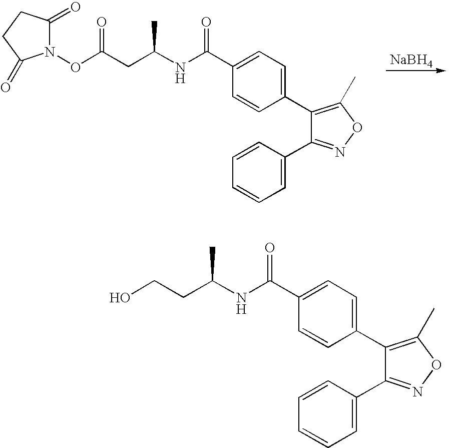

N-{(1R)-3-[(2,5-Dioxopyrrolidin-1-yl)oxy]-1-methyl-3-oxopropyl}-4-(5-methyl-3-phenylisoxazol-4-yl)benzamide (100 mg, 0.22 mmol) was dissolved in THF (5 ml), and sodium borohydride (16 mg, 0.42 mmol) was added thereto at 0° C., and the mixture was stirred at room temperature for 3 hours. A saturated aqueous ammonium chloride solution (2 ml) was added to the reaction mixture at 0° C. and the mixture was extracted with ethyl acetate. The extract was washed with brine, dried over sodium sulfate, and concentrated under reduced pressure. The residue was purified by silica gel column chromatography (hexane:ethyl acetate=4:1->1:1) to give N-[(1R)-3-hydroxy-1-methylpropyl]-4-(5-methyl-3-phenylisoxazol-4-yl)benzamide (75 mg, 99%) as a solid.

MS: 351 [M+H]⁺, APCI (MeOH)

### Example 232

The following compound was prepared by carrying out a reaction and a treatment in a manner similar to Example 231.

TABLE 45

| | | Physical |
|---|---|---|
| Example | Chemical structure | constant, etc. |
| 232 | 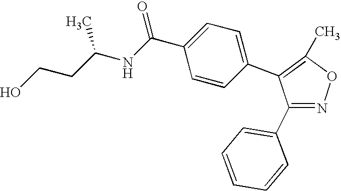 | MS: 351 [M + H]⁺, APCI (MeOH) |

### Example 233

The following compound was prepared by carrying out a reaction and a treatment in a manner similar to Example 44 (2).

TABLE 46

| | | Physical |
|---|---|---|
| Example | Chemical structure | constant, etc. |
| 233 | 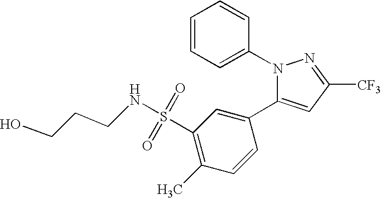 | MS: 440 [M + H]⁺, ESI |

### Examples 234-248

The following compounds were prepared by carrying out a reaction and a treatment in a manner similar to Example 48.

TABLE 47

| | | Physical |
|---|---|---|
| Example | Chemical structure | constant, etc. |
| 234 | 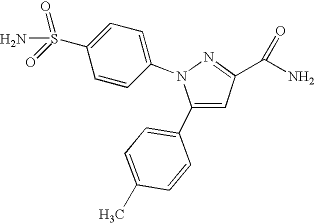 | MS: 357 [M + H]⁺, APCI (MeOH) |
| 235 | 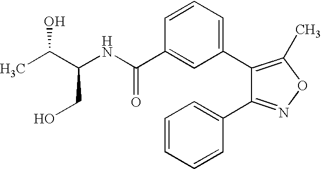 | MS: 367 [M + H]⁺, APCI (MeOH) |
| 236 | 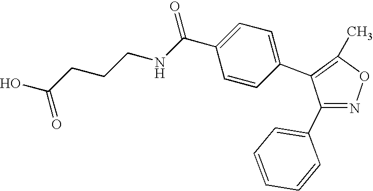 | MS: 363 [M − H]⁻, ESI |
| 237 | 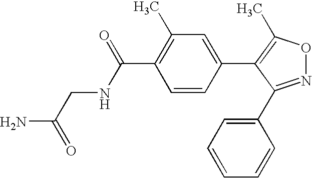 | MS: 350 [M + H]⁺, APCI |
| 238 | 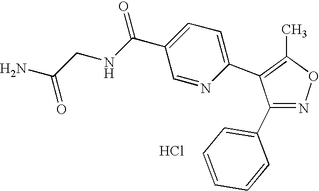 | MS: 337 [M + H]⁺, APCI |
| 239 | 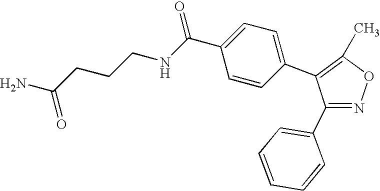 | MS: 364 [M + H]⁺, APCI |
| 240 | 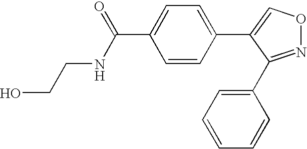 | MS: 309 [M + H]⁺, APCI |
| 241 | 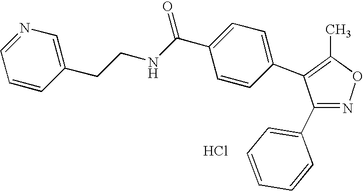 | MS: 384 [M + H]⁺, APCI |
| 242 | 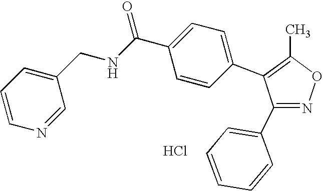 | MS: 370 [M + H]⁺, APCI |
| 243 | 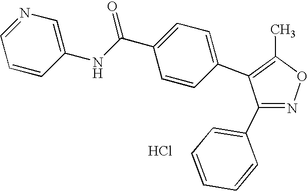 | MS: 356 [M + H]⁺, APCI |
| 244 | 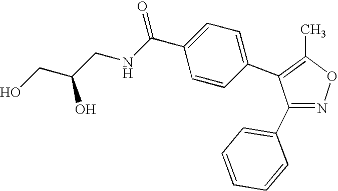 | MS: 353 [M + H]⁺, APCI |
| 245 | 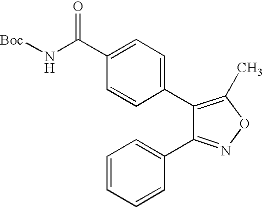 | MS: 377 [M − H]⁻, ESI |
| 246 | 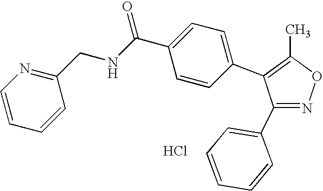 | MS: 370 [M + H]⁺, APCI |
| 247 | 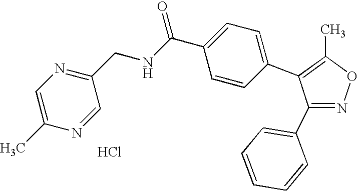 | MS: 385 [M + H]⁺, APCI |
| 248 | 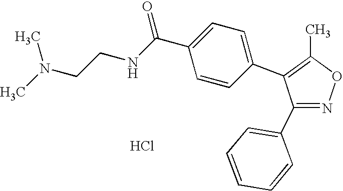 | MS: 350 [M + H]⁺, APCI |

### Example 249

The following compound was prepared by carrying out a reaction and a treatment in a manner similar to Example 121.

TABLE 48

| | | Physical |
|---|---|---|
| Example | Chemical structure | constant, etc. |
| 249 | 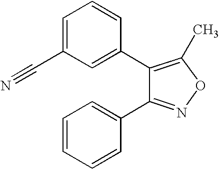 | MS: 261 [M + H]⁺, APCI (MeOH) |

### Example 250

(1)

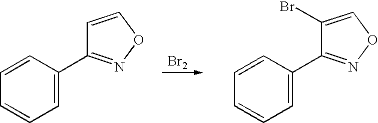

Bromine (6.0 ml, 117.1 mmol) was added to a solution of 3-phenylisoxazole (840 mg, 5.787 mmol) in acetic acid (15.0 ml) and the mixture was heated at 90° C. for 96 hours while stirring. The reaction mixture was cooled and poured into a saturated aqueous sodium bicarbonate solution and the mixture was extracted with ethyl acetate. The organic layer was washed with a 15% aqueous sodium thiosulfate solution and subsequently with brine, dried over sodium sulfate, and concentrated under reduced pressure. The residue was purified by silica gel column chromatography (hexane:ethyl acetate=100:0->90:10) to give 4-bromo-3-phenylisoxazole (1290 mg, 99%) as a solid.

MS: 224/226 [M+H]⁺, APCI

(2) The following compound was prepared by carrying out a reaction and a treatment in a manner similar to Example 121 using 4-bromo-3-phenylisoxazole.

TABLE 49

| | | Physical |
|---|---|---|
| Example | Chemical structure | constant, etc. |
| 250 (2) | 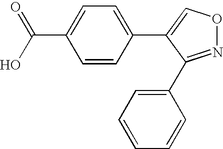 | MS: 264 [M − H]⁻, ESI |

### Examples 251 and 252

The following compounds were prepared by carrying out a reaction and a treatment in a manner similar to Example 135.

TABLE 50

| | | Physical |
|---|---|---|
| Example | Chemical structure | constant, etc. |
| 251 | 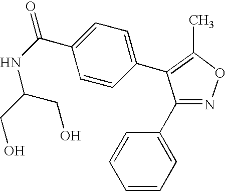 | MS: 353 [M + H]⁺, ESI |
| 252 | 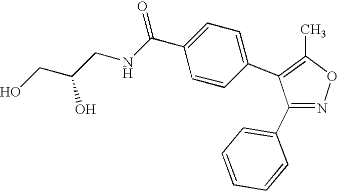 | MS: 353 [M + H]⁺, APCI |

### Examples 253 and 254

The following compounds were prepared by carrying out a reaction and a treatment in a manner similar to Example 150.

TABLE 51

| | | Physical |
|---|---|---|
| Example | Chemical structure | constant, etc. |
| 253 | 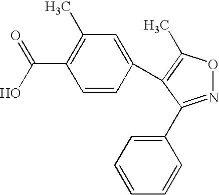 | MS: 292 [M − H]⁻, ESI |
| 254 | 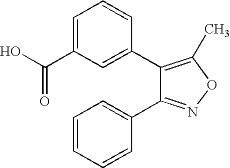 | MS: 278 [M − H]⁻, ESI (MeOH) |

### Examples 255 and 256

The following compounds were prepared by carrying out a reaction and a treatment in a manner similar to Example 151.

TABLE 52

| | | Physical |
|---|---|---|
| Example | Chemical structure | constant, etc. |
| 255 | 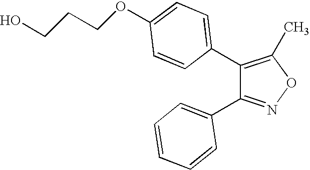 | MS: 310 [M + H]⁺, APCI (MeOH) |
| 256 | 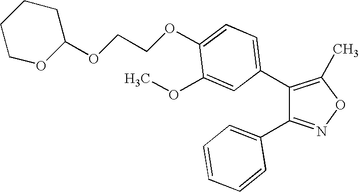 | MS: 427 [M + NH₄]⁺, APCI |

### Example 257

The following compound was prepared by carrying out a reaction and a treatment in a manner similar to Example 152.

TABLE 53

| Exam- | | Physical |
|---|---|---|
| ple | Chemical structure | constant, etc. |
| 257 | 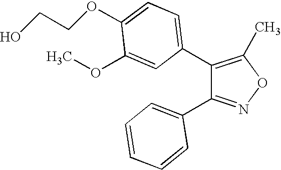 | MS: 326 [M + H]⁺, APCI |

### Example 258

The following compound was prepared by carrying out a reaction and a treatment in a manner similar to Example 153.

TABLE 54

| | | Physical |
|---|---|---|
| Example | Chemical structure | constant, etc. |
| 258 | 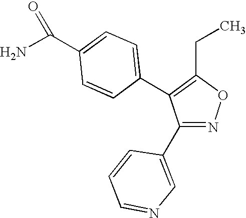 | MS: 294 [M + H]⁺, APCI |

### Examples 259 and 260

The following compounds were prepared by carrying out a reaction and a treatment in a manner similar to Example 156.

TABLE 55

| Exam- | | Physical |
|---|---|---|
| ple | Chemical structure | constant, etc. |
| 259 | 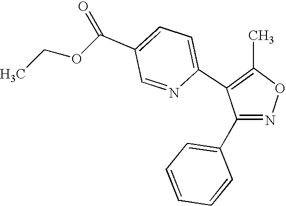 | MS: 309 [M + H]⁺, APCI |
| 260 | 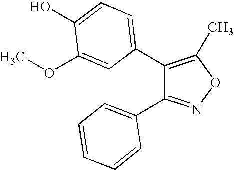 | MS: 282 [M + H]⁺, APCI |

### Example 261

The following compound was prepared by carrying gut a reaction and a treatment in a manner similar to Example 158.

TABLE 56

| | | Physical |
|---|---|---|
| Example | Chemical structure | constant, etc. |
| 261 | 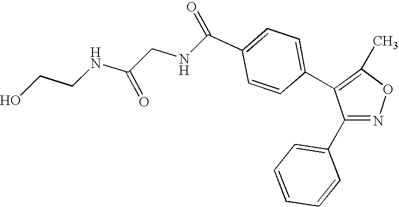 | MS: 380 [M + H]⁺, APCI |

### Example 262

The following compound was prepared by carrying out a reaction and a treatment in a manner similar to Example 162 (2) and (3).

TABLE 57

| Exam- | | Physical |
|---|---|---|
| ple | Chemical structure | constant, etc. |
| 262 | 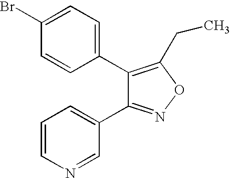 | MS: 329/331 [M + H]⁺, APCI |

### Example 263

The following compound was prepared by carrying out a reaction and a treatment in a manner similar to Example 162 (4).

TABLE 58

| | | Physical |
|---|---|---|
| Example | Chemical structure | constant, etc. |
| 263 | 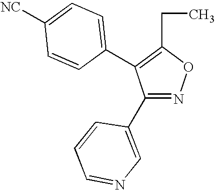 | MS: 276 [M + H]⁺, APCI |

### Examples 264-266

The following compounds were prepared by carrying out a reaction and a treatment in a manner similar to Example 48.

TABLE 59

| | | Physical |
|---|---|---|
| Example | Chemical structure | constant, etc. |
| 264 | 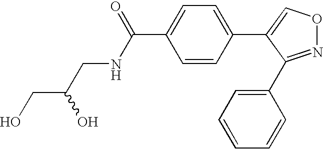 | MS: 339 [M + H]⁺, APCI |
| 265 | 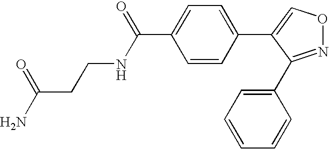 | MS: 336 [M + H]⁺, APCI |
| 266 | 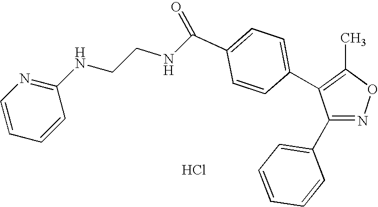 | MS: 399 [M + H]⁺, APCI |

### Examples 267 and 268

The following compounds were prepared by carrying out a reaction and a treatment in a manner similar to Example 150.

TABLE 60

| | | Physical |
|---|---|---|
| Example | Chemical structure | constant, etc. |
| 267 | 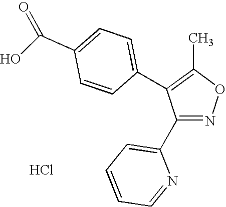 | MS: 279 [M − H]⁻, ESI |
| 268 | 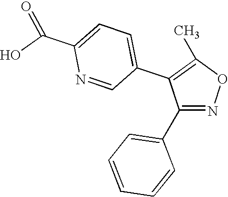 | MS: 279 [M − H]⁻, ESI |

### Examples 269-271

The following compounds were prepared by carrying out a reaction and a treatment in a manner similar to Example 153.

TABLE 61

| Exam- | | Physical |
|---|---|---|
| ple | Chemical structure | constant, etc. |
| 269 | 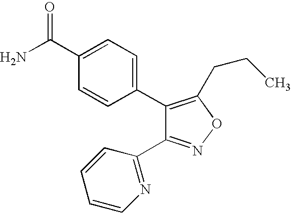 | MS: 308 [M + H]⁺, APCI |
| 270 | 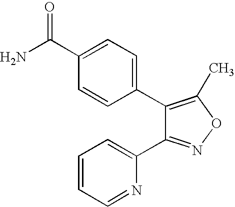 | MS: 280 [M + H]⁺, APCI |
| 271 | 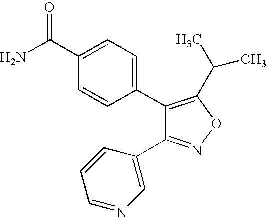 | MS: 308 [M + H]⁺, APCI |

### Examples 272 and 273

The following compounds were prepared by carrying out a reaction and a treatment in a manner similar to Example 156.

TABLE 62

| Exam- | | Physical |
|---|---|---|
| ple | Chemical structure | constant, etc. |
| 272 | 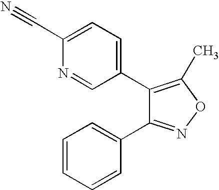 | MS: 262 [M + H]⁺, ESI |
| 273 | 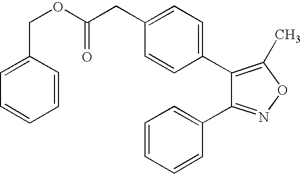 | MS: 384 [M + H]⁺, APCI |

### Example 274

The following compound was prepared by carrying out a reaction and a treatment in a manner similar to Example 162 (5).

TABLE 63

| | | Physical |
|---|---|---|
| Example | Chemical structure | constant, etc. |
| 274 | 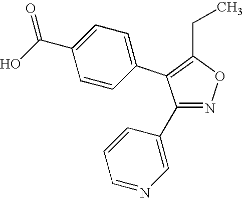 | MS: 293 [M − H]⁻, ESI |

### Examples 275-289

The following compounds were prepared by carrying out a reaction and a treatment in a manner similar to Example 48.

TABLE 64

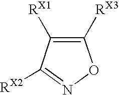

| | | | | Physical |
|---|---|---|---|---|
| | | | | constant, |
| Example | R^{x1} | R^{x2} | R^{x3} | etc. |
| 275 | 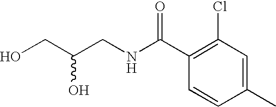 | Ph | Me | MS: 387/389 [M + H]⁺, APCI |
| 276 | 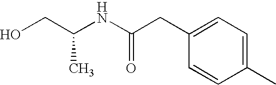 | Ph | Me | MS: 351 [M + H]⁺, APCI |
| 277 | 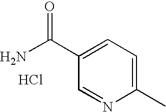 | Ph | Me | MS: 280 [M + H)⁺, APCI |
| 278 | 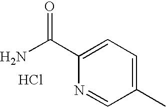 | Ph | Me | MS: 280 [M + H], APCI |
| 279 | 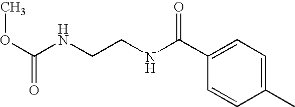 | 3-Pyri- dyl | Me | MS: 381 [M + H]⁺, APCI |
| 280 | 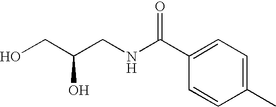 | 3-Pyri- dyl | Me | MS: 354 [M + H]⁺, APCI |
| 281 | 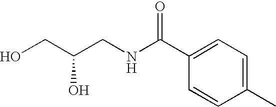 | 3-Pyri- dyl | Me | MS: 354 [M + H]⁺, APCI |
| 282 | 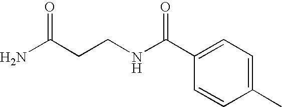 | 3-Pyri- dyl | Me | MS: 351 [M + H]⁺, APCI |
| 283 | 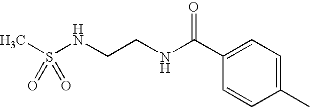 | 3-Pyri- dyl | Me | MS: 401 [M + H]⁺, APCI |
| 284 | 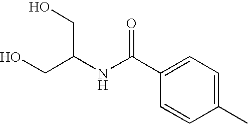 | 3-Pyri- dyl | Et | MS:368 [M + H]⁺, APCI |
| 285 | 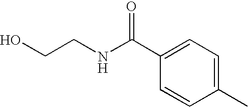 | 2-Pyri- dyl | Me | MS: 324 [M + H]⁺, APCI |
| 286 | 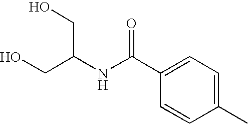 | 2-Pyri- dyl | Me | MS: 354 [M + H]⁺, APCI |
| 287 | 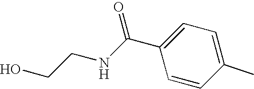 | 3-Pyri- dyl | Et | MS: 338 [M + H]⁺, ESI |
| 288 | 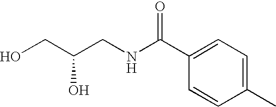 | 3-Pyri- dyl | Et | MS: 368 [M + H]⁺, ESI |
| 289 | 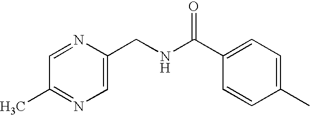 | 3-Pyri- dyl | Et | MS: 400 [M + H]⁺, ESI |

### Example 290

The following compound was prepared by carrying out a reaction and a treatment in a manner similar to Example 70.

TABLE 65

| Exam- | | Physical |
|---|---|---|
| ple | Chemical structure | constant, etc. |
| 290 | 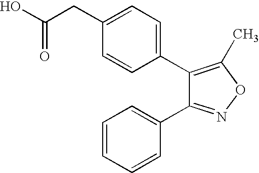 | MS: 294 [M + H]⁺, APCI |

### Example 291

(1)

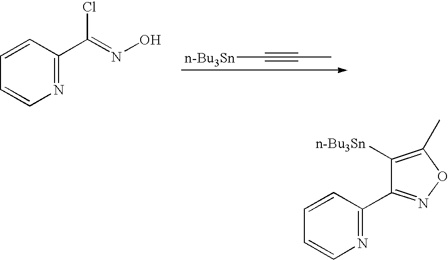

To a solution of 2-pyridinecarbohydroxymoyl chloride (500 mg, 3.19 mmol) and tributyl(1-propyn-1-yl)stannane (1.94 ml, 6.38 mmol) in THF (10 ml) was added dropwise triethylamine (1.00 ml, 7.18 mmol) over a period of 15 minutes under ice-cooling. After the mixture was allowed to stand overnight and the temperature thereof was returned to room temperature, the reaction mixture was concentrated and diluted with hexane. The insolubles were removed by filtration, and the filtrate was concentrated under reduced pressure. The obtained residue was purified by basic silica gel column chromatography (hexane) to give 2-[5-methyl-4-(tributylstannyl)isoxazol-3-yl]pyridine (554 mg, 39%) as an oil.

MS: 447/449/451 [M+H]⁺, APCI (MeOH)

(2)

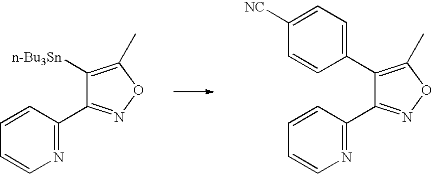

A solution of 2-[5-methyl-4-(tributylstannyl)isoxazol-3-yl]pyridine (100 mg, 0.223 mmol), 4-bromobenzonitrile (61 mg, 0.325 mmol), tetrakis(triphenylphosphine)palladium (30 mg, 0.026 mmol) and cuprous iodide (5 mg, 0.026 mmol) in dioxane (3 ml) was refluxed under heating overnight. After allowed to cool, the reaction mixture was diluted with ethyl acetate and a saturated aqueous potassium fluoride solution was added thereto, and the mixture was stirred at room temperature for 2 hours. After the precipitate was removed by filtration, the filtrate was washed with brine, dried over sodium sulfate, and concentrated under reduced pressure. The residue was purified by silica gel column chromatography (hexane:ethyl acetate=95:5->65:35) to give 2-[5-methyl-4-(4-cyanophenyl)isoxazol-3-yl]pyridine (48 mg, 83%) as powders.

Ms: 262 [M+H]⁺, APCI (MeOH)

### Examples 292 and 293

(1) The following compounds were prepared by carrying out a reaction and a treatment in a manner similar to Example 291 (1) and (2).

TABLE 66

| | | Physical |
|---|---|---|
| Example | Chemical structure | constant, etc. |
| 292 | 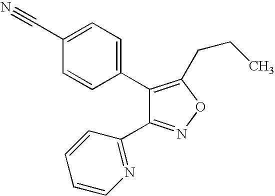 | MS: 290 [M + H]⁺, APCI |
| 293 | 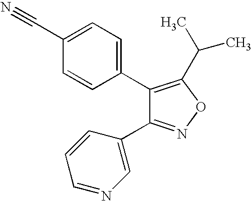 | MS: 290 [M + H]⁺, APCI |

### Examples 294-299

The following compounds were prepared by carrying out a reaction and a treatment in a manner similar to Example 48.

TABLE 67

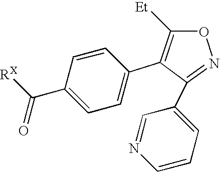

| | | Physical |
|---|---|---|
| Example | R^{x} | constant, etc. |
| 294 | 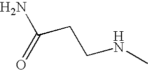 | MS: 365 [M + H]⁺, ESI |
| 295 | 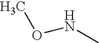 | MS: 324 [M + H]⁺, ESI |
| 296 | 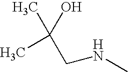 | MS: 366 [M + H]⁺, ESI |
| 297 | 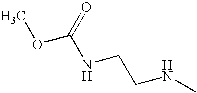 | MS: 395 [M + H]⁺, ESI |
| 298 | 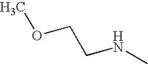 | MS: 352 [M + H]⁺, ESI |
| 299 | 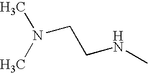 | MS: 365 [M + H]⁺, ESI |

### Example 300

(1)

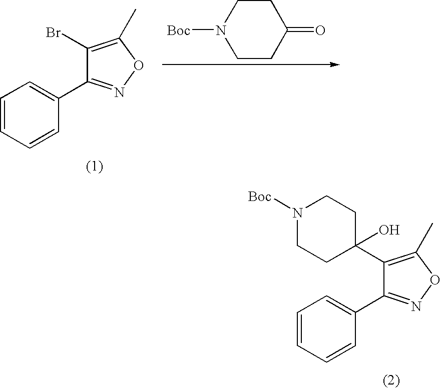

A 1.6N n-butyl lithium hexane solution (21 ml, 33 mmol) was added to a solution of 4-bromo-5-methyl-3-phenylisoxazole (7.14 g, 0.30 mmol) in THF (100 ml) under dry ice-acetone cooling. After the mixture was stirred at the same temperature for 30 minutes, 1-tert-butoxycarbonylpiperidin-4-one (6.8 g, 34.3 mmol) was added thereto. The reaction mixture was warmed to room temperature, water was added thereto and the mixture was extracted with ethyl acetate. The organic layer was washed with brine, dried over sodium sulfate and concentrated under reduced pressure. The residue was purified by silica gel column chromatography (hexane:ethyl acetate=2:1), and the obtained residue was crystallized from diethyl ether-hexane to give tert-butyl 4-hydroxy-4-(5-methyl-3-phenylisoxazol-4-yl)piperidine-1-carboxylate (5.83 g, 54%).

MS: 359 [M+H]⁺, APCI (MeOH)

(2)

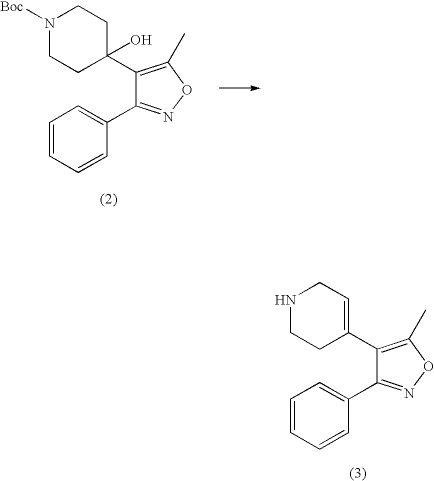

tert-Butyl 4-hydroxy-4-(5-methyl-3-phenylisoxazol-4-yl)piperidine-1-carboxylate was dissolved in a PPSE dichlorobenzene solution (40 ml) and the mixture was heated at 140° C. overnight. After cooling, the reaction mixture was poured into water, neutralized with sodium bicarbonate, and extracted with ethyl acetate. The organic layer was washed with brine, dried over sodium sulfate, and concentrated under reduced pressure. The residue was purified by NH silica gel column chromatography (ethyl acetate) to give 4-(5-methyl-3-phenylisoxazol-4-yl)-1,2,3,6-tetrahydropyridine (193 mg, 16%).

MS: 241 [M+H]⁺, APCI (MeOH)

Preparation of the PPSE Dichlorobenzene Solution:

A mixture of bis(trimethylsilyl)ether (0.5 L) and o-dichlorobenzene (1 L) was heated to 150° C. and diphosphorous pentoxide (200 g) was added portionwise thereto. The mixture was left to stand at the same temperature for 10 minutes, and the obtained solution was cooled to room temperature to give the PPSE dichlorobenzene solution.

(3)

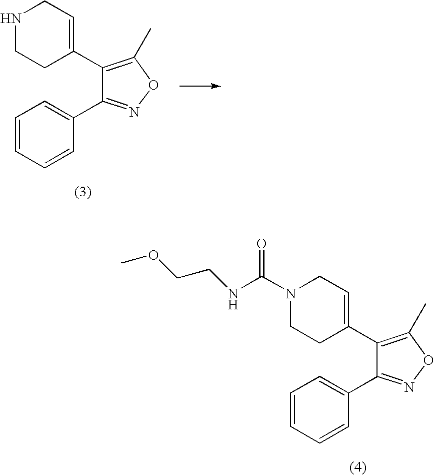

To a solution of triphosgen (41.2 mg, 0.14 mmol) in methylene chloride (2 ml) was added 2-methoxy-1-ethylamine (37 μl, 0.42 mmol) and followed by triethylamine (120 μl, 0.84 mmol) under ice-cooling, and the mixture was stirred at the same temperature for 15 minutes. 4-(5-methyl-3-phenylisoxazol-4-yl)-1,2,3,6-tetrahydropyridine (95 mg, 0.39 mmol) was added thereto and the mixture was stirred at room temperature for 2 days. The reaction mixture was purified by silica gel column chromatography (chloroform:methanol=100:0->97:3) to give N-(2-methoxyethyl)-4-(5-methyl-3-phenylisoxazol-4-yl)-3,6-dihydropyridine-1(2H)-carboxamide (121 mg, 91%).

MS: 342 [M+H]⁺, APCI (MeOH)

### Example 301

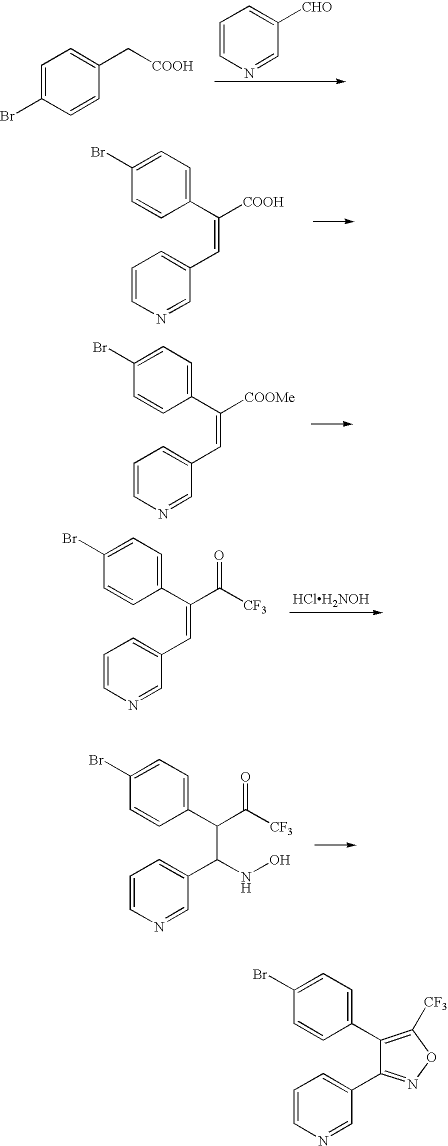

(1) 4-Bromophenylacetic acid (15.0 g, 7.0 mmol), nicotine-aldehyde (7.47 g, 7.0 mmol) and triethylamine (9.7 ml, 7.0 mmol) were dissolved in acetic anhydride (60 ml) and the mixture was refluxed under heating for 20 hours. The mixture was cooled to 110° C., and water (30 ml) was gradually added thereto while stirring. After 30 minutes, the reaction mixture was cooled to room temperature, and precipitated crystals were collected by filtration, washed with water and diethyl ether, and dried to give (2E)-2-(4-bromophenyl)-3-pyridin-3-ylacrylic acid (11.1 g, 52%) as a solid.

MS: 302/304[M−H]⁻, ESI (MeOH)

(2) To methanol (150 ml) was added dropwise thionyl chloride (2.9 ml, 4.0 mmol) at −10° C., and the mixture was stirred for 20 minutes. After (2E)-2-(4-bromophenyl)-3-pyridin-3-ylacrylic acid (11.0 g, 3.6 mmol) was added thereto, the mixture was gradually warmed to room temperature, and then, stirred at 70° C. for 14 hours. Methanol was removed under reduced pressure, a saturated aqueous sodium bicarbonate solution was added thereto, and the mixture was extracted with ethyl acetate. The extract was washed with brine, dried over sodium sulfate, and concentrated under reduced pressure. The residue was purified by silica gel column chromatography (hexane:ethyl acetate=9:1->1:1) to give methyl (2E)-2-(4-bromophenyl)-3-pyridin-3-ylacrylate (8.78 g, 76%) as a liquid.

MS: 318/320[M+H)⁺, APCI (MeOH)

(3) To a solution of methyl (2E)-2-(4-bromophenyl)-3-pyridin-3-ylacrylate (14.14 g, 44.4 mmol) and cesium fluoride (70 mg, 0.46 mmol) in DME (100 ml) was added (trifluoromethyl)trimethylsilane (8.23 ml, 55.7 mmol) at room temperature. After an hour, 4N hydrochloric acid (100 ml) was added thereto and the mixture was stirred at room temperature for 3 hours. A saturated aqueous sodium bicarbonate solution was added thereto, and the mixture was extracted with ethyl acetate. The extract was washed with brine, dried over sodium sulfate, and concentrated under reduced pressure to give quantitatively a crude product of (3E)-3-(4-bromophenyl)-1,1,1-trifluoro-4-pyridin-3-ylbut-3-en-2-one as a liquid.

MS: 356/358[M+H]⁺, APCI (MeOH)

(4) A mixture of (3E)-3-(4-bromophenyl)-1,1,1-trifluoro-4-pyridin-3-ylbut-3-en-2-one (15.0 g, 42.1 mmol), hydroxylamine hydrochloride (3.22 g, 46.3 mmol) and sodium acetate (3.80 g, 46.3 mmol) in anhydrous ethanol (400 ml) was refluxed under heating for an hour. After the mixture was cooled and concentrated under reduced pressure, ethyl acetate was added thereto. The mixture was washed with brine, dried over sodium sulfate, and concentrated under reduced pressure. The residue was purified by silica gel column chromatography (hexane:ethyl acetate=9:1->1:1) to give 3-(4-bromophenyl)-1,1,1-trifluoro-4-(hydroxyamino)-4-pyridin-3-ylbutan-2-one (8.80 g, 54%) as powders.

MS: 389/391[M+H]⁺, APCI (MeOH)

(5) A mixture of 3-(4-bromophenyl)-1,1,1-trifluoro-4-(hydroxyamino)-4-pyridin-3-ylbutan-2-one (8.80 g, 22.6 mmol), iodine (5.74 g, 22.6 mmol), potassium iodide (37.5 g, 226 mmol) and sodium bicarbonate (19.0 g, 226 mmol) in a mixed solvent of THF (200 ml) and water (100 ml) was refluxed under light-shielding for 7 hours, and the mixture was further stirred at room temperature overnight. After the mixture was concentrated under reduced pressure, ethyl acetate was added thereto. The mixture was washed with brine, dried over sodium sulfate, and concentrated under reduced pressure. The residue was purified by silica gel column chromatography (hexane:ethyl acetate=9:1->1:1) to give 3-[4-(4-bromophenyl)-5-(trifluoromethyl)isoxazol-3-yl]pyridine (6.05 g, 73%) as oil.

MS: 369/371[M+H]⁺, APCI (MeOH)

### Example 302

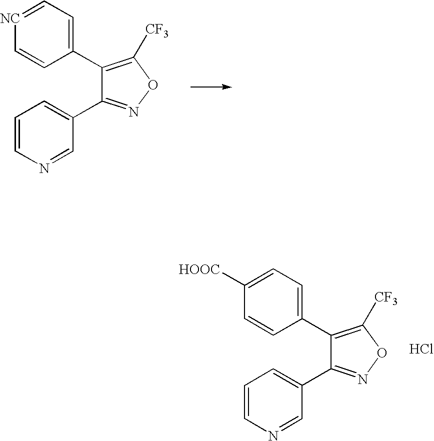

A mixture of 4-[3-pyridin-3-yl-5-(trifluoromethyl)isoxazol-4-yl]benzonitrile (1.60 g, 5.08 mmol) in 6N hydrochloric acid (10 ml) was refluxed under heating for 4 days. The mixture was cooled and concentrated under reduced pressure to give 4-[3-pyridin-3-yl-5-(trifluoromethyl)isoxazol-4-yl]benzoic acid hydrochloride (1.71 g, 91%) as powders.

MS: 333[M−H]⁻, ESI (MeOH)

### Example 303

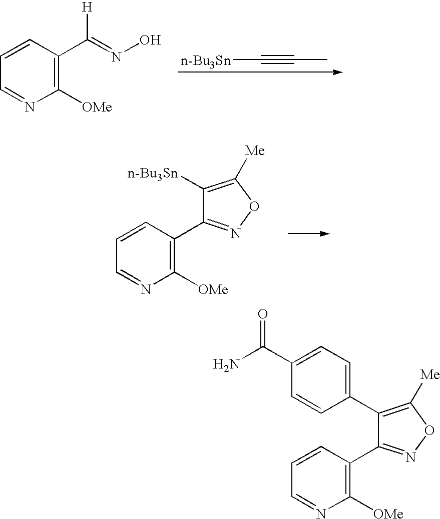

(1) To a suspension of 2-methoxynicotinealdehyde oxime (1350 mg, 8.87 mmol), tributyl(1-propyn-1-yl)stannane (2.97 ml, 9.76 mmol), potassium bicarbonate (1780 mg, 17.74 mmol) and water (one drop) in ethyl acetate (10 ml) was added N-chlorosuccinimide (1320 mg, 9.76 mmol) under ice-cooling, and the mixture was stirred at room temperature overnight. The reaction mixture was diluted with ethyl acetate, insolubles were removed by filtration with basic silica gel, and the filtrate was concentrated under reduced pressure. The residue was purified by silica gel column chromatography (hexane:ethyl acetate=50:1->30:1) to give 2-methoxy-3-[5-methyl-4-(tributylstannyl)isoxazol-3-yl]pyridine (1820 mg, 43%) as oil.

MS: 477/479/481[M+H]⁺, APCI (MeOH)

(2) A solution of 2-methoxy-3-[5-methyl-4-(tributylstannyl)isoxazol-3-yl]pyridine (373 mg, 0.778 mmol), 4-bromobenzamide (120 mg, 0.600 mmol) and dichlorobis(triphenylphosphine)palladium (II) (42 mg, 0.060 mmol) in 1,4-dioxane (6 ml) was refluxed under heating overnight. The reaction mixture was cooled and diluted with ethyl acetate. A 10% potassium fluoride aqueous solution was added thereto, and the mixture was stirred at room temperature for 30 minutes. Precipitates were removed by filtration and the filtrate was washed with brine, dried over sodium sulfate, and concentrated under reduced pressure. The residue was purified by silica gel column chromatography (ethyl acetate:methanol=99:1->93:7) to give 4-[3-(2-methoxypyridin-3-yl)-5-methylisoxazol-4-yl]-benzamide (64 mg, 35%) as a foam.

MS: 310[M+H]⁺, APCI (MeOH)

### Example 304 to 341

The following compounds were prepared by carrying out a reaction and a treatment in a manner similar to the above-mentioned examples using the corresponding starting compound.

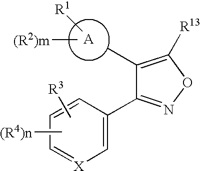

| Example | Ring A | R¹ | R² | m | R³ | R⁴ | n | R¹³ | X | MS:m/z |
|---|---|---|---|---|---|---|---|---|---|---|
| 304 | 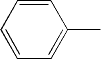 | 4-CON(CH₂CH₂OH)₂ | 3-Cl | 1 | H | — | 0 | Me | CH | 401/403 [M + H]+,APCI |
| 305 | 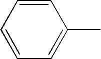 | 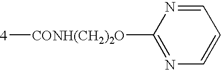 | — | 0 | H | — | 0 | Me | CH | 401 [M + H]+,APCI |
| 306 | 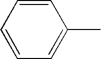 | 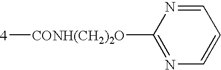 | — | 0 | H | — | 0 | Et | N | 416 [M + H]+,ESI |
| 307 | 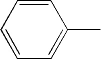 | 4-CONH(CH₂)₂CH₃ | — | 0 | H | — | 0 | Et | N | 336 [M + H]+,ESI |
| 308 | 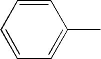 | 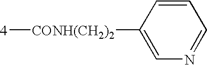 | — | 0 | H | — | 0 | Et | N | 399 [M + H]+,ESI |
| 309 | 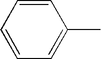 | 4-CONMe₂ | — | 0 | H | — | 0 | Et | N | 322 [M + H]+,APCI |
| 310 | 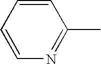 | 5-CONHC(Me)₂CH₂OH | — | 0 | H | — | 0 | Me | CH | 352 [M + H]+,ESI |
| 311 | 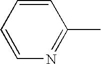 | 5-CONH(CH₂)₂OH | — | 0 | H | — | 0 | Me | CH | 324 [M + H]+,ESI |
| 312 | 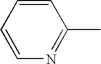 | 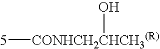 | — | 0 | H | — | 0 | Me | CH | 338 [M + H]+,ESI |
| 313 | 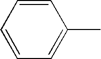 | 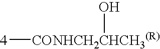 | — | 0 | H | — | 0 | H | CH | 323 [M + H]+,ESI |
| 314 | 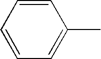 | 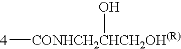 | — | 0 | H | — | 0 | H | CH | 339 [M + H]+,ESI |
| 315 | 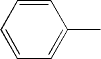 | 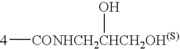 | — | 0 | H | — | 0 | H | CH | 339 [M + H]+,ESI |

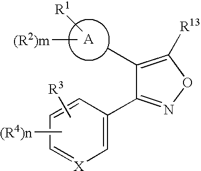

| Example | Ring A | R¹ | R² | m | R³ | R⁴ | n | R¹³ | X | MS:m/z |
|---|---|---|---|---|---|---|---|---|---|---|
| 316 | 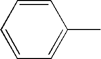 | 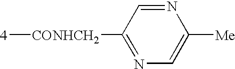 | — | 0 | H | — | 0 | H | CH | 371 [M + H]+,ESI |
| 317 | 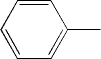 | 4-CONH₂ | — | 0 | 4-CH₃ | — | 0 | Me | CH | 293 [M + H]+,APCI |
| 318 | 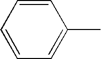 | 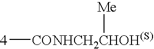 | — | 0 | 4-CH₃ | — | 0 | Me | CH | 351 [M + H]+,APCI |
| 319 | 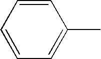 | 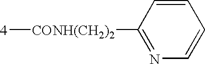 | — | 0 | 4-CH₃ | — | 0 | Me | CH | 398 [M + H]+,APCI |
| 320 | 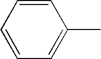 | 4-CONH(CH₂)₂OH | — | 0 | 4-CH₃ | — | 0 | Me | CH | 337 [M + H]+,APCI |
| 321 | 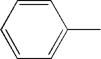 | 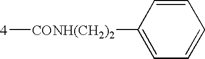 | — | 0 | H | — | 0 | Me | N | 384 [M + H]+,APCI |
| 322 | 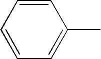 | 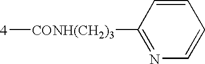 | — | 0 | H | — | 0 | Me | N | 399 [M + H]+,ESI |
| 323 | 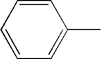 | 4-CONHCH₂CH(OH)CF₃ | — | 0 | H | — | 0 | Me | CH | 391 [M + H]+,APCI |
| 324 | 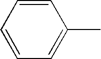 | 4-CONHCH₂CH(OH)CH₂OMe | — | 0 | H | — | 0 | Me | CH | 367 [M + H]+,APCI |
| 325 | 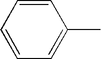 | 4-CONH₂ | — | 0 | H | 4-F | 1 | Me | CH | 297 [M + H]+,APCI |
| 326 | 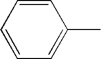 | 4-CONHCH₂CONH₂ | — | 0 | H | 4-F | 1 | Me | CH | 354 [M + H]+,APCI |
| 327 | 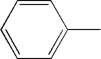 | H | 4-CN | 1 | H | — | 0 | CF₃ | N | 316 [M + H]+,APCI |
| 328 | 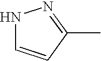 | H | — | 0 | H | — | 0 | Me | CH | (commercially available) |
| 329 | 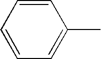 | 4-CONHCH₂CH(OH)CH₂OMe | — | 0 | H | — | 0 | Me | N | 368 [M + H]+,APCI |

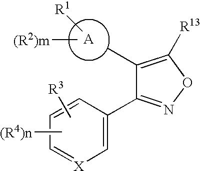

| Example | Ring A | R¹ | R² | m | R³ | R⁴ | n | R¹³ | X | MS:m/z |
|---|---|---|---|---|---|---|---|---|---|---|
| 330 | 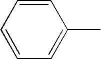 | 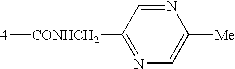 | — | 0 | H | 4-F | 1 | Me | CH | 403 [M + H]+,APCI |
| 331 | 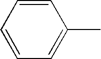 | 4-CONH₂ | — | 0 | H | — | 0 | CF₃ | N | 334 [M + H]+,APCI |
| 332 | 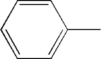 | 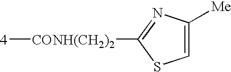 | — | 0 | H | — | 0 | Me | CH | 404 [M + H]+,APCI |
| 333 | 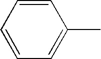 | 4-CONHOMe | — | 0 | H | — | 0 | Me | CH | 309 [M + H]+,APCI |
| 334 | 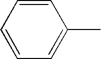 | 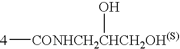 | — | 0 | H | — | 0 | CF₃ | N | 408 [M + H]+,APCI |
| 335 | 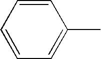 | 4-CONH₂ | — | 0 | 5-Me | — | 0 | Me | N | 294 [M + H]+,APCI |
| 336 | 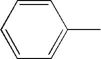 | 4-CONH₂ | — | 0 | 2-OMe | — | 0 | Me | CH | 309 [M + H]+,APCI |
| 337 | 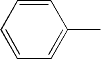 | 4-CONH₂ | — | 0 | 3-OMe | — | 0 | Me | CH | 309 [M + H]+,APCI |
| 338 | 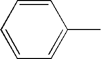 | 4-COOH | — | 0 | 4-Me | — | 0 | Me | CH | 292 [M − H]−,ESI |
| 339 | 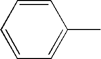 | 4-COOMe | — | 0 | 4-Me | — | 0 | Me | CH | 308 [M + H]+,APCI |
| 340 | 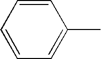 | 4-COOH | — | 0 | H | 4-F | 1 | Me | CH | 296 [M − H]−,ESI |
| 341 | 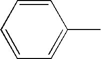 | H | 4-CN | 1 | H | 4-F | 1 | Me | CH | 279 [M + H]+,APCI |

### Example 342

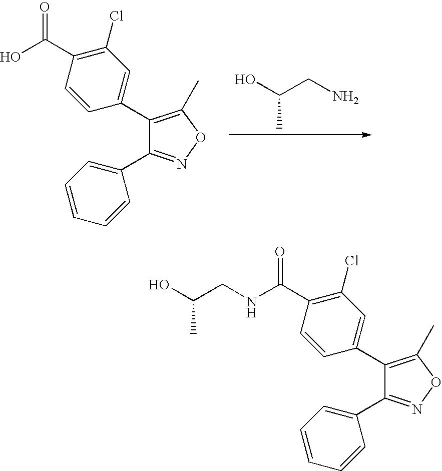

A mixture of 2-chloro-4-(5-methyl-3-phenylisoxazol-4-yl)benzoic acid (78 mg, 0.25 mmol), (S)-1-amino-2-propanol (0.039 ml, 0.49 mmol), N-hydroxybenzotriazole (40 mg, 0.30 mmol) and 1-ethyl-3-(3′-dimethylaminopropyl)carbodiimide hydrochloride (100 mg; 0.52 mmol) in DMF (2 ml) was stirred at room temperature overnight. Water was added to the reaction mixture and the mixture was extracted with ethyl acetate. The extract was washed with water and brine, dried over sodium sulfate, and concentrated under reduced pressure. The residue was purified by silica gel column chromatography (hexane:ethyl acetate=7:3->1:0) to give 2-chloro-N-[(2S)-2-hydroxypropyl]-4-(5-methyl-3-phenylisoxazol-4-yl)benzamide (84 mg, 91%) as powders.

MS: 371/373 [M+H]⁺, APCI

### Example 343

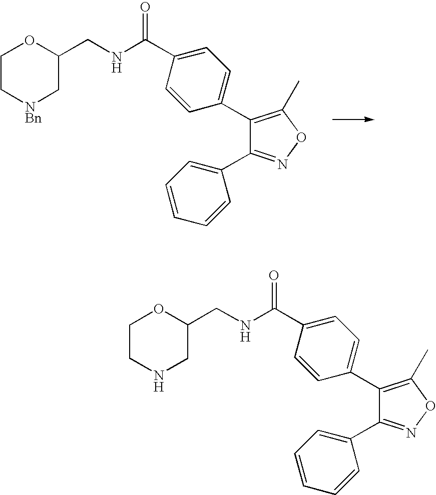

3-Chloroperoxybenzoic acid (85% purity, 13 mg, 0.0640 mmol) and 1 M aqueous iron(II) chloride solution (0.030 ml, 0.030 mmol) was added successively to a solution of N-[(4-benzyl-morpholin-2-yl)methyl]-4-(5-methyl-3-phenylisoxazol-4-yl)-benzamide (30 mg, 0.0642 mmol) in dichloromethane (2 ml) under ice-acetone cooling. Reaction mixture was stirred for three days at room temperature, and a dilute aqueous ammonia solution was added thereto. The mixture was extracted with chloroform, and the extract was washed with brine, dried over sodium sulfate, and concentrated under reduced pressure. The residue was purified by NH silica gel column chromatography (chloroform:methanol=100:0->95:5) to give 4-(5-methyl-3-phenylisoxazol-4-yl)-N-(morpholin -2-ylmethyl)benzamide (13 mg, 53%) as caramels.

MS: 378 [M+H]⁺, APCI

### Example 344

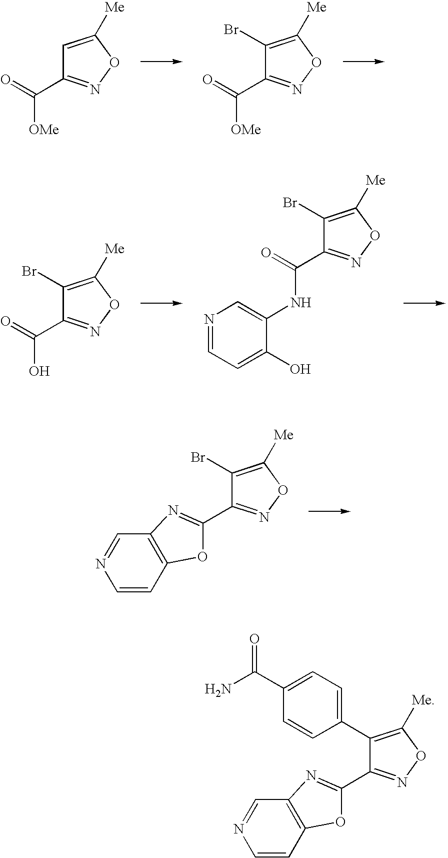

(1) Bromine (49.4 g, 309 mmol) was added to a solution of methyl 5-methylisoxazole-3-carboxylate (30 g, 206 mmol) in chloroform (103 ml) at room temperature and the mixture was refluxed for 3 hours. After cooling, the reaction mixture was poured into a saturated aqueous potassium carbonate-saturated aqueous sodium thiosulfate and extracted with chloroform twice. The combined organic layer was washed with brine, dried over sodium sulfate, and concentrated under reduced pressure. The residue was purified by silica gel column chromatography (hexane:ethyl acetate=10:1) to give methyl 4-bromo-5-methylisoxazole-3-carboxylate (27.3 g, 60%) as a solid.

MS: 220/222 [M+H]⁺, APCI (MeOH)

(2) An aqueous sodium hydroxide solution (4N, 20.5 ml, 81.8 mmol) was added to a solution of methyl 4-bromo-5-methylisoxazole-3-carboxylate (15.0 g, 68.2 mmol) in methanol (150 ml) under ice-cooling, and the mixture was stirred at room temperature for 2 hours. Hydrochloric acid (6N, 13.6 ml, 81.8 mmol) was added and the mixture was concentrated under reduced pressure. The residue was dissolved in ethyl acetate, dried over sodium sulfate, filtered through a celite pad and concentrated under reduced pressure. The residue was triturated with diisopropyl ether to give 4-bromo-5-methylisoxazole-3-carboxylic acid (12.1 g, 86%) as a solid.

MS: 160/162 [M−CO₂−H]⁻, ESI (MeOH)

(3) Oxalyl chloride (616 mg, 4.86 mmol) was added to a suspension of 4-bromo-5-methylisoxazole-3-carboxylic acid (500 mg, 2.43 mmol) and DMF (17.7 mg, 0.243 mmol) in chloroform (10 ml) at room temperature and the mixture was stirred for 30 minutes. The reaction mixture was concentrated under reduced pressure. The residue was dissolved in chloroform (5 ml) and then the mixture was added to a suspension of 3-amino-4-hydroxypyridine hydrochloride (533 mg, 3.64 mmol) and pyridine (960 mg, 12.1 mmol) in chloroform (5 ml) under ice-cooling. The mixture was stirred at room temperature for 2 hours. Water was added and the mixture was extracted with ethyl acetate twice. The combined organic layer was dried over sodium sulfate, filtered and concentrated under reduced pressure. The residue was triturated with diisopropyl ether to give 4-bromo-N-(4-hydroxypyridin-3-yl)-5-methylisoxazole-3-carboxamide (574 mg, 79%) as a solid.

MS: 298/300 [M+H]⁺, APCI (MeOH)

(4) A mixture of 4-bromo-N-(4-hydroxypyridin-3-yl)-5-methylisoxazole-3-carboxamide (572 mg, 1.92 mmol) and polyphospholic acid (5.72 g) was stirred at 150° C. for an hour. After cooling, the reaction mixture was diluted with water, basified with 15% aqueous sodium hydroxide solution and extracted with ethyl acetate twice. The combined organic layer was dried over sodium sulfate, filtered and concentrated under reduced pressure. The residue was purified by silica gel column chromatography (chloroform:ethyl acetate=5:1) to give 2-(4-bromo-5-methylisoxazol-3-yl)(1,3]oxazolo[4,5-c]pyridine (331 mg, 61%) as a solid.

MS: 280/282 [M+H]⁺, APCI (MeOH)

(5) 2-(4-bromo-5-methylisoxazol-3-yl)[1,3]oxazolo[4,5-c]pyridine was reacted and treated in a manner similar to example 121 to give 4-[5-methyl-3-([1,3]oxazolo[4,5-c]-pyridin-2-yl)isoxazol-4-yl]benzamide.

MS: 321 [M+H]⁺, APCI

### Example 345

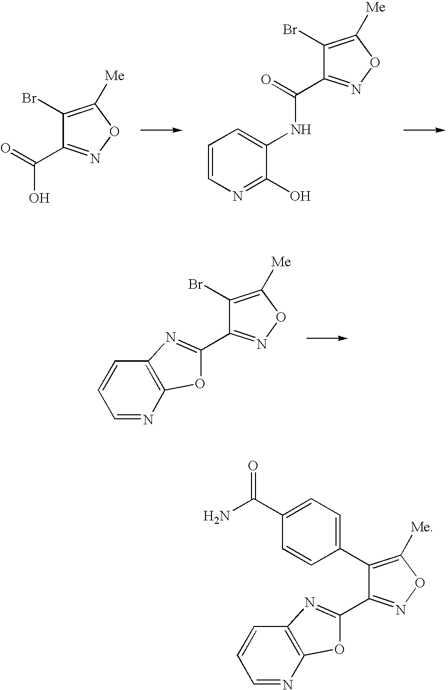

(1) 4-Bromo-5-methylisoxazol-3-carboxylic acid was reacted and treated in a manner similar to example 344 (3) using 3-amino-2-hydroxypyridine, to give 4-bromo-N-(2-hydroxypyridin-3-yl)-5-methylisoxazole-3-carboxamide.

(2) A mixture of 4-bromo-N-(2-hydroxypyridin-3-yl)-5-methylisoxazole-3-carboxamide (512 mg, 1.72 mmol) and phosphoryl chloride (8.42 g) was refluxed overnight. After cooling, the reaction mixture was poured into water, basified with 15% aqueous sodium hydroxide solution and extracted with ethyl acetate. The organic layer was washed with brine, dried over sodium sulfate, filtered and concentrated under reduced pressure. The residue was purified by silica gel column chromatography (hexane:ethyl acetate=93:7->17:3) to give 2-(4-bromo-5-methylisoxazol-3-yl)[1,3]oxazolo[5,4-b]pyridine (101 mg, 21%) as a solid.

MS: 280/282 [M+H]⁺, APCI (MeOH)

(3) 2-(4-Bromo-5-methylisoxazol-3-yl)[1,3]oxazolo[5,4-b]-pyridine was reacted and treated in a manner similar to example 121 to give 4-[5-methyl-3-([1,3]oxazolo[5,4-b]-pyridin-2-yl)isoxazol-4-yl]benzamide.

MS: 321 [M+H]⁺, APCI

### Example 346

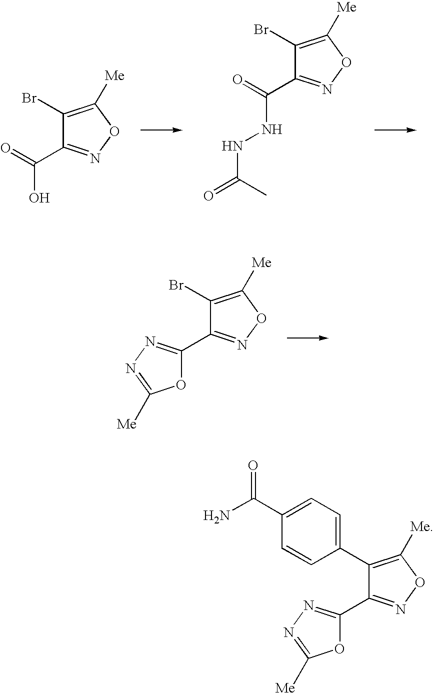

(1) 4-Bromo-5-methylisoxazol-3-carboxylic acid was reacted and treated in a manner similar to example 344 (3) using acetylhydrazine, to give N′-acetyl-4-bromo-5′-methylisoxazole-3-carbohydrazide.

(2) Triethylamine (586 mg, 5.79 mmol) was added to a solution of N′-acetyl-4-bromo-5-methylisoxazole-3-carbohydrazide (506 mg, 1.931 mmol) and 2-Chloro-1,3-dimethylimidazolinium chloride (490 mg, 2.90 mmol) in chloroform (15 ml) under ice-cooling. The mixture was stirred at room temperature overnight and refluxed for 5 hours. After cooling, water was added and the mixture was extracted with chloroform. The organic layer was concentrated under reduced pressure. The residue was dissolved in 1,2-dichloroethane (5 ml) and the mixture was refluxed overnight. After cooling, the reaction mixture was concentrated under reduced pressure. The residue was purified by silica gel column chromatography (hexane:ethyl acetate=9:1->4:1) to give 2-(4-bromo-5-methylisoxazol-3-yl)-5-methyl-1,3,4-oxadiazole (62 mg, 13%) as a solid.

MS: 244/246 [M+H]⁺, APCI (MeOH)

(3) 2-(4-Bromo-5-methylisoxazol-3-yl)-5-methyl-1,3,4-oxadiazole was reacted and treated in a manner similar to example 121 to give 4-[5-methyl-3-(5-methyl-1,3,4-oxadiazol-2-yl)isoxazol-4-yl]benzamide.

MS: 285 [M+H]⁺, APCI

### Example 347

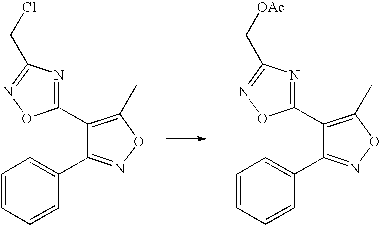

To a solution of 3-(Chloromethyl)-5-(5-methyl-3-phenylisoxazol-4-yl)-1,2,4-oxadiazole (100 mg, 0.36 mmol) in DMF (2 ml) was added sodium acetate (44 mg, 0.54 mmol) at room temperature, and the mixture was stirred at 60° C. for 3 hours. After the mixture was cooled, water was added thereto, and the mixture was extracted with ethyl acetate. The extract was washed with brine, dried over sodium sulfate, and concentrated under reduced pressure. The residue was purified by silica gel column chromatography (hexane:ethyl acetate=10:1) to give [5-(5-methyl-3-phenylisoxazol-4-yl)-1,2,4-oxadiazol-3-yl]methyl acetate (87.4 mg, 81%) as a solid.

Ms: 300 [M+H]⁺, APCI (MeOH)

### Example 348

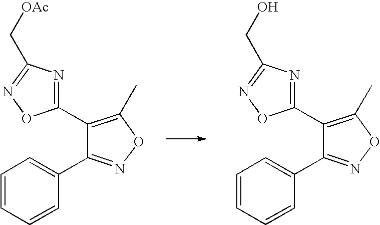

To a solution of [5-(5-methyl-3-phenylisoxazol-4-yl)-1,2,4-oxadiazol-3-yl]methyl acetate (86.2 mg, 0.29 mmol) in methanol was added water (0.5 ml) and followed by potassium carbonate (199 mg, 1.44 mmol) at room temperature, and the mixture was stirred at the same temperature for an hour. The reaction mixture was poured into water, extracted with ethyl acetate. The organic layer was washed with brine, dried over sodium sulfate, and concentrated under reduced pressure. The residue was purified by silica gel column chromatography (hexane:ethyl acetate=5:1->1:1) to give [5-(5-methyl-3-phenylisoxazol-4-yl)-1,2,4-oxadiazol-3-yl]methanol (67.8 mg, 91%) as a solid.

Ms: 258 [M+H]⁺, APCI (MeOH)

### Example 349

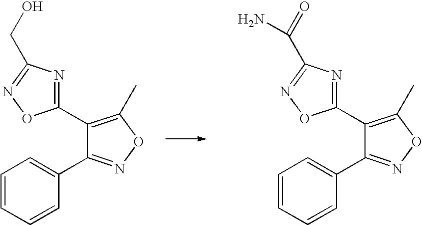

To a solution of [5-(5-methyl-3-phenylisoxazol-4-yl)-1,2,4-oxadiazol-3-yl]methanol (67.8 mg, 0.26 mmol) in acetone were added 2,2,6,6-tetramethylpiperidin-1-oxyl (41 mg, 0.26 mmol) and sodium hydrogen carbonate (50 mg) in water (1 ml), followed by potassium bromide (3.6 mg, 0.03 mmol) at room temperature. The solution was cooled to 0° C. and aqueous sodium hypochlorite solution (0.9 ml, 0.58 mmol) was added thereto and the mixture was stirred at room temperature overnight. The reaction mixture was concentrated under reduced pressure. The residue was poured into 10% hydrochloric acid and extracted with ethyl acetate. The organic layer was dried over sodium sulfate, concentrated under reduced pressure. The residue was dissolved in dichloromethane. Oxalyl chloride (0.026 ml, 0.29 mmol) and DMF (0.015 ml) were added thereto at room temperature. The reaction mixture was stirred at the same temperature for an hour, poured into 30% aqueous ammonia (2 ml), and extracted with chloroform. The organic layer was dried over sodium sulfate and concentrated under reduced pressure. The residue was purified by silica gel column chromatography (hexane:ethyl acetate=5:1->1:1) to give 5-(5-methyl-3-phenylisoxazol-4-yl)-1,2,4-oxadiazole-3-carboxamide (33.1 mg, 47%) as a solid.

Ms: 271 [M+H]⁺, APCI (MeOH)

### Example 350

The following compound was prepared in a manner similar to example 344 or 345 using the corresponding starting compound.

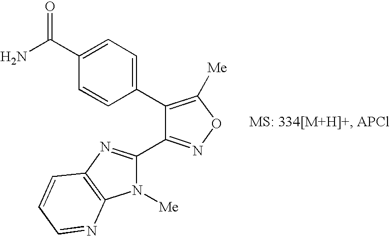

### Example 351 to 419

The following compounds were prepared by carrying out a reaction and a treatment in a manner similar to the above-mentioned examples using the corresponding starting compound.

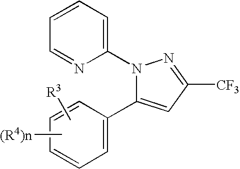

| Example | R³ | R⁴ | n | MS:m/z |
|---|---|---|---|---|
| 351 | 4-Me | — | 0 | 304[M + H]+, APCI |
| 352 | 3-SO₂NH(CH₂)₂CH₃ | 4-Me | 1 | 425[M + H]+, ESI |

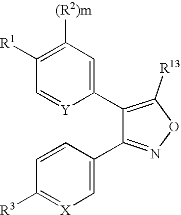

| Example | R¹ | R² | m | R³ | R¹³ | X | Y | MS:m/z |
|---|---|---|---|---|---|---|---|---|
| 353 | —CONHCH₂CH(OH)CH₃(R) | — | 0 | H | Me | CH | CH | 337[M + H]+, ESI |
| 354 | —CONH(CH₂)₂OCH₃ | — | 0 | H | Me | CH | CH | 337[M + H]+, APCI |
| 355 | 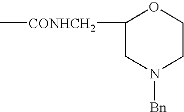 | — | 0 | H | Me | CH | CH | 468[M + H]+, APCI |
| 355 | —COOH | F | 1 | H | Me | CH | CH | 296[M − H]−, ESI |
| 357 | —CONHOH | — | 0 | H | Me | CH | N | 296[M + H]+, APCI |
| 358 | —CONH(CH₂)₂CONH₂ | — | 0 | Me | Me | CH | CH | 364[M + H]+, APCI |
| 359 | —CNHCH₂CH(OH)CH₂(S) | — | 0 | H | Et | N | CH | 352[M + H]+, ESI |

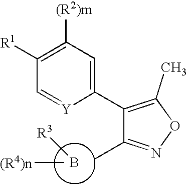

| Example | Salt | R¹ | R² | m | Y | Ring B | R³ | R⁴ | n | MS:m/z |
|---|---|---|---|---|---|---|---|---|---|---|
| 360 | | —CONH₂ | — | 0 | CH | 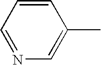 | 2-Me | — | 0 | 294[M + H]+, APCI |
| 361 | HCl | —CONH₂ | — | 0 | N | 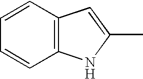 | 1-Me | 3-Cl | 1 | 367/369[M + H]+, APCI |
| 362 | HCl | —CONH₂ | — | 0 | N | 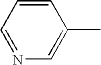 | 6-OMe | — | 0 | 311[M + H]+, APCI |
| 363 | | —CONH₂ | — | 0 | CH | 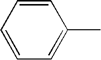 | H | 3-F | 1 | 297[M + H]+, APCI |
| 364 | | —CONH₂ | — | 0 | CH | 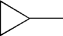 | H | — | 0 | 243[M + H]+, APCI |
| 365 | | —CONH₂ | — | 0 | CH | 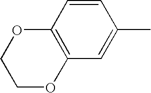 | H | — | 0 | 337[M + H]+, APCI |
| 366 | HCl | —CONH₂ | — | 0 | CH | 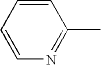 | 5-OMe | — | 0 | 310[M + H]+, APCI |
| 367 | HCl | —CONH₂ | — | 0 | CH | 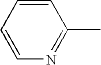 | 4-OMe | — | 0 | 310[M + H]+, APCI |
| 368 | | —COOEt | — | 0 | CH | 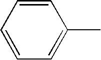 | H | 3-F | 1 | 326[M + H]+, APCI |
| 369 | | —CONH₂ | — | 0 | CH | 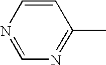 | H | — | 0 | 281[M + H]+, APCI |
| 370 | | —CONH₂ | — | 0 | CH | 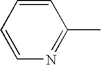 | 5-Me | — | 0 | 294[M + H]+, APCI |
| 371 | | —CONH₂ | — | 0 | CH | 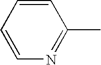 | 4-Me | — | 0 | 294[M + H]+, APCI |

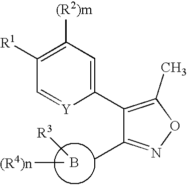

| Example | Salt | R¹ | R² | m | Y | Ring B | R³ | R⁴ | n | MS:m/z |
|---|---|---|---|---|---|---|---|---|---|---|
| 372 | | —CONH₂ | — | 0 | CH | 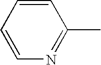 | H | 3-F | 1 | 298[M + H]+, APCI |
| 373 | | —CONH₂ | — | 0 | CH | 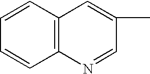 | H | — | 0 | 330[M + H]+, APCI |
| 374 | | —CONH₂ | — | 0 | CH | 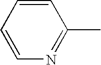 | H | 5-F | 1 | 298[M + H]+, APCI |
| 375 | | —CONH₂ | — | 0 | N | 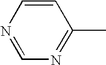 | H | — | 0 | 282[M + H]+, APCI |
| 376 | | —CONH₂ | — | 0 | N | 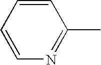 | 6-OMe | — | 0 | 311[M + H]+, APCI |
| 377 | | —CONH₂ | — | 0 | N | 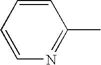 | H | 3-F | 1 | 299[M + H]+, APCI |
| 378 | | —CONH₂ | — | 0 | N | 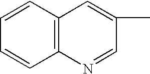 | H | — | 0 | 331[M + H]+, APCI |
| 379 | | —CONH₂ | — | 0 | N | 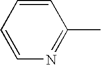 | H | 5-F | 1 | 299[M + H]+, APCI |
| 380 | | —CONH₂ | — | 0 | N | 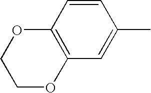 | H | — | 0 | 338[M + H]+, APCI |
| 381 | | —CONH₂ | — | 0 | N | 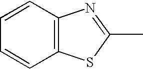 | H | — | 0 | 337[M + H]+, APCI |
| 382 | | —CONH₂ | — | 0 | N | 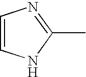 | 1-Me | — | 0 | 284[M + H]+, APCI |

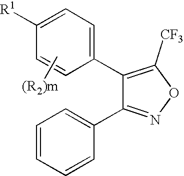

| Example | R¹ | R² | m | MS:m/z |
|---|---|---|---|---|
| 383 | H | 4-Br | 1 | 368/370[M + H]+, APCI |
| 384 | —CN | — | 0 | 347[M + H + MeOH]+, APCI |
| 385 | —COOH | — | 0 | 332[M − H]−, ESI |

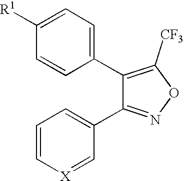

| Ex- | | | | |
|---|---|---|---|---|
| ample | Salt | R¹ | X | MS:m/z |
| 386 | | —CONHCH₂CH(OH)CH₂OH (S) | CH | 407[M + H]+, |
| | | | | APCI |
| 387 | | —CONHCH(CH₂OH)₂ | CH | 407[M + H]+, |
| | | | | APCI |
| 388 | HCl | —CONH(CH₂)₄CONH₂ | N | 433[M + H]+, |
| | | | | APCI |
| 389 | HCl | —CONH(CH₂)₃CONH₂ | N | 419[M + H]+, |
| | | | | APCI |
| 390 | HCl | —CONH(CH₂)₂CONH₂ | N | 405[M + H]+, |
| | | | | APCI |
| 391 | | —CONH₂ | CH | 333[M + H]+, |
| | | | | APCI |

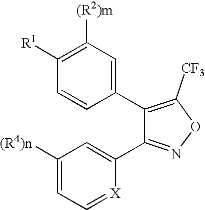

| Example | Salt | R¹ | R² | m | X | R⁴ | n | MS:m/z |
|---|---|---|---|---|---|---|---|---|
| 392 | | —CONH(CH₂)₂OH | —Cl | 1 | CH | — | 0 | 357/359[M + H]+, APCI |
| 393 | HCl | 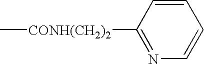 | —Cl | 1 | CH | — | 0 | 418/420[M + H]+, APCI |
| 394 | | 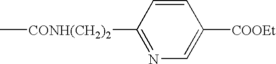 | — | 0 | CH | — | 0 | 456[M + H]+, APCI |
| 395 | | 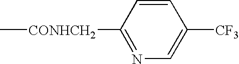 | — | 0 | CH | — | 0 | 438[M + H]+, APCI |
| 396 | | 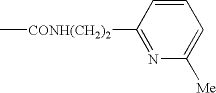 | — | 0 | CH | — | 0 | 398[M + H]+, APCI |
| 397 | | —CONHCH₂C(CH₃)₂OH | — | 0 | CH | — | 0 | 351[M + H]+, APCI |
| 398 | | 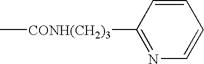 | — | 0 | CH | — | 0 | 398[M + H]+, APCI |
| 399 | | 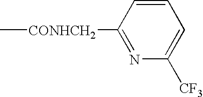 | — | 0 | CH | — | 0 | 438[M + H]+, APCI |
| 400 | | 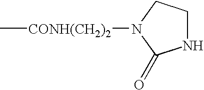 | — | 0 | CH | — | 0 | 391[M + H]+, APCI |
| 401 | | 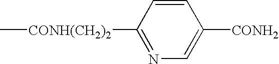 | — | 0 | CH | — | 0 | 427[M + H]+, APCI |
| 402 | | —CONHCH₂CH(OH)CH₃(R) | — | 0 | N | — | 0 | 338[M + H]+, APCI |
| 403 | | —COOH | — | 0 | CH | F | 1 | 296[M + H]+, APCI |
| 404 | | 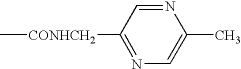 | — | 0 | CH | F | 1 | 403[M + H]+, APCI |

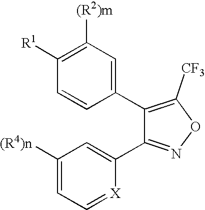

| Example | R¹ | R² | m | X | R⁴ | n | MS:m/z |
|---|---|---|---|---|---|---|---|
| 405 | —CONHCH₂CH(OH)CH₂OH (S) | — | 0 | CH | F | 1 | 371[M + H]+, APCI |
| 406 | —CONHCH₂CH(OH)CH₃ (R) | — | 0 | CH | F | 1 | 355[M + H]+, APCI |
| 407 | —CONHCH(CH₃)CH₂OH (R) | — | 0 | CH | F | 1 | 355[M + H]+, APCI |
| 408 | —CONHCH(CH₃)CH₂OH (S) | — | 0 | CH | F | 1 | 355[M + H]+, APCI |
| 409 | —CONH(CH₂)₂CONH₂ | — | 0 | CH | F | 1 | 368[M + H]+, APCI |
| 410 | —CONHCH₂CONH₂ | — | 0 | CH | F | 1 | 354[M + H]+, APCI |
| 411 | —CONHCH₂CH(OH)CH₂OH (R) | F | 1 | CH | — | 0 | 371[M + H]+, APCI |
| 412 | —CONHCH₂CH(OH)CH₂OH (S) | F | 1 | CH | — | 0 | 371[M + H]+, APCI |
| 413 | —CONHCH₂CH(OH)CH₃ (S) | F | 1 | CH | — | 0 | 355[M + H]+, APCI |
| 414 | —CONHCH₂CH(OH)CH₃ (R) | F | 1 | CH | — | 0 | 355[M + H]+, APCI |
| 415 | —CONHCH(CH₂OH)₂ | F | 1 | CH | — | 0 | 371[M + H]+, APCI |
| 416 | —CON(CH₃)₂ | F | 1 | CH | — | 0 | 325[M + H]+, APCI |
| 417 | —CON(CH₂CH₂OH)₂ | F | 1 | CH | — | 0 | 385[M + H]+, APCI |

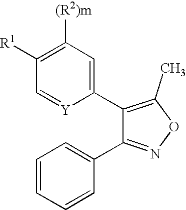

| Example | R¹ | R² | m | Y | MS:m/z |
|---|---|---|---|---|---|
| 418 | —SO₂NH₂ | — | 0 | N | 316[M + H]+, APCI |
| 419 | —COOMe | F | 1 | CH | 355[M + H]+, APCI |

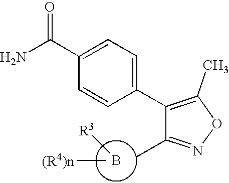

| No. | Ring B | R³ | R⁴ | n |
|---|---|---|---|---|
| 1 | 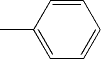 | H | 2-F | 1 |
| 2 | 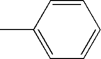 | 2-Me | — | 0 |
| 3 | 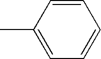 | 3-Me | — | 0 |
| 4 | 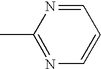 | H | — | 0 |
| 5 | 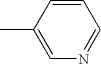 | 4-Me | — | 0 |
| 6 | 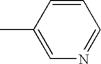 | 4-OMe | — | 0 |
| 7 | 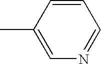 | H | 5-F | 1 |
| 8 | 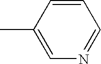 | H | 5-Cl | 1 |
| 9 | 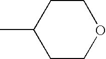 | H | — | 0 |
| 10 | 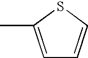 | H | 5-Cl | 1 |
| 11 | 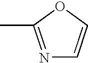 | H | — | 0 |
| 12 | 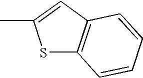 | 6-Me | — | 0 |
| 13 | 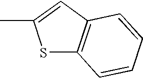 | 6-OMe | — | 0 |
| 14 | 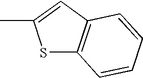 | 5-Me | — | 0 |
| 15 | 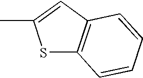 | 5-OMe | — | 0 |
| 16 | 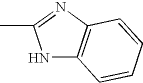 | 1-Me | — | 0 |
| 17 | 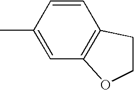 | H | — | 0 |
| 18 | 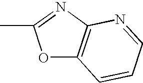 | H | — | 0 |
| 19 | 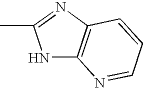 | H | — | 0 |
| 20 | 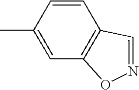 | H | — | 0 |
| 21 | 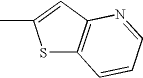 | H | — | 0 |
| 22 | 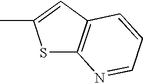 | H | — | 0 |
| 23 | 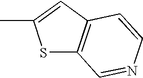 | H | — | 0 |
| 24 | 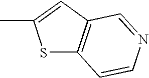 | H | — | 0 |
| 25 | 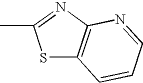 | H | — | 0 |
| 26 | 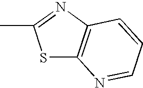 | H | — | 0 |
| 27 | 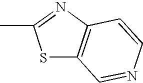 | H | — | 0 |
| 28 | 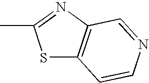 | H | — | 0 |

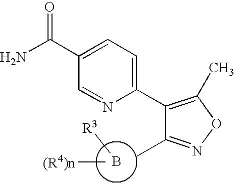

| No. | Ring B | R³ | R⁴ | n |
|---|---|---|---|---|
| 29 | 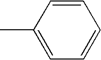 | H | 2-F | 1 |
| 30 | 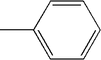 | H | 3-F | 1 |
| 31 | 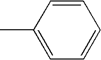 | 2-Me | — | 0 |
| 32 | 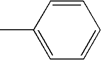 | 3-Me | — | 0 |
| 33 | 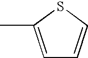 | H | 5-Cl | 1 |
| 34 | 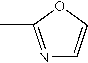 | H | — | 0 |
| 35 | 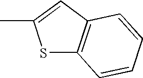 | 6-Me | — | 0 |
| 36 | 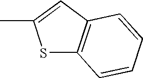 | 6-OMe | — | 0 |
| 37 | 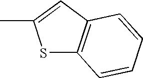 | 5-Me | — | 0 |
| 38 | 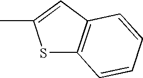 | 5-OMe | — | 0 |
| 39 | 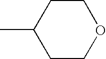 | H | — | 0 |
| 40 | 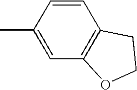 | H | — | 0 |

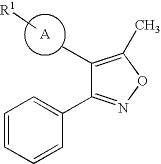

| No. | Ring A | R¹ |
|---|---|---|
| 41 | 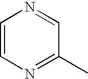 | 5-CONH₂ |
| 42 | 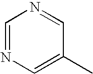 | 2-CONH₂ |
| 43 | 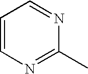 | 5-CONH₂ |
| 44 | 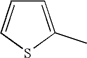 | 4-CONH₂ |
| 45 | 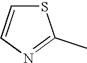 | 5-CONH₂ |
| 46 | 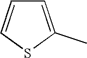 | 4-SO₂NH₂ |
| 47 | 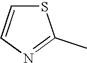 | 5-SO₂NH₂ |
| 48 | 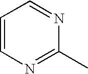 | 5-SO₂NH₂ |

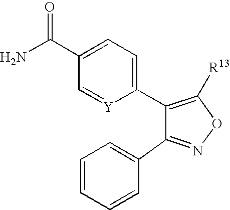

| No. | Y | R¹³ |
|---|---|---|
| 49 | N | CF₃ |
| 50 | CH | OH |
| 51 | CH | OMe |

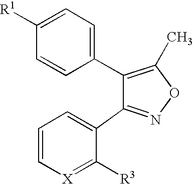

| No. | R¹ | X | R³ |
|---|---|---|---|
| 52 | —CONHCH₂CH(OH)CH₂OH | CH | Me |
| 53 | —CONHCH₂CH(OH)CH₂OH | N | Me |
| 54 | —CONHCH₂CH(OH)CH₂OH | N | OMe |
| 55 | —CONHCH₂CH(OH)CH₃ | CH | Me |
| 56 | —CONHCH₂CH(OH)CH₃ | N | Me |
| 57 | —CONHCH₂CH(OH)CH₃ | N | OMe |
| 58 | —CONH(CH₂)₂OH | CH | Me |
| 59 | —CONH(CH₂)₂OH | N | Me |
| 60 | —CONH(CH₂)₂OH | N | OMe |

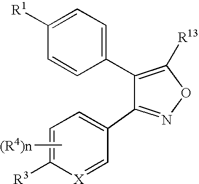

| No. | R¹ | X | R³ | R⁴ | n | R¹³ |
|---|---|---|---|---|---|---|
| 61 | —CONHCH(CH₂OH)₂ | CH | H | 4-F | 1 | CF₃ |
| 62 | —CONHCH(CH₂OH)₂ | CH | Me | — | 0 | CF₃ |
| 63 | —CONHCH(CH₂OH)₂ | CH | Me | — | 0 | Me |
| 64 | —CONHCH₂CH(OH)CH₂OH | CH | H | 4-Cl | 1 | Me |
| 65 | —CONHCH₂CH(OH)CH₂OH | CH | H | 4-F | 1 | H |
| 66 | —CONHCH₂CH(OH)CH₂OH | N | H | — | 0 | H |
| 67 | —CONHCH₂CH(OH)CH₂OH | CH | H | 4-Cl | 1 | H |
| 68 | —CONHCH₂CH(OH)CH₂OH | CH | Me | — | 0 | H |
| 69 | —CONH(CH₂)₂OH | CH | H | 4-F | 1 | Me |
| 70 | 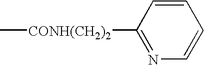 | CH | H | 4-F | 1 | Me |
| 71 | —CONHCH₂CH(OH)CH₂OH | CH | H | 4-F | 1 | Me |
| 72 | —CONHCH(CH₂OH)₂ | CH | H | 4-F | 1 | Me |

### Experimental Example 1

Relaxation Effect on Potassium-Induced Contraction of Isolated Rabbit Urinary Bladder

Urinary bladder was isolated from Male NZW rabbits (body weight: 2.0-3.5 kg) and immersed in ice-cold Krebs-bicarbonate solution (in mM: 118 NaCl, 4.7 KCl, 2.55 CaCl₂, 1.18 MgSO₄, 1.18 KH₂PO₄, 24.88 NaHCO₃ and 11.1 glucose). The urinary bladder was cut into longitudinal strips (5 mm length, 3-4 mm width) after mucosal layer was removed.

Preparations were mounted in organ baths containing 10 ml of Krebs solution maintained at 37° C. and gassed with 95% O₂/5% CO₂. Accordingly, preparations were stretched with an initial tension of 2.0±1.0 g, and changes in isometric tension were measured by force-displacement transducer. The preparations were pre-contracted by changing organ-bath solution into high-K⁺ (30 mM) Krebs solution (in mM: 118 NaCl, 4.7 KCl, 2.55 CaCl₂, 1.18 MgSO₄, 1.18 KH₂PO₄, 24.88 NaHCO₃ and 11.1 glucose).

After stable tension was obtained, compounds were added into organ baths cumulatively (10⁻⁸ M-10⁻⁴ M). The effects of compounds were expressed as a percentage of the maximum relaxation produced by 1O⁻⁴ M papaverine as 100%. 50% relaxation concentration (IC₅₀) was calculated and IC₅₀ value range (μM) of compounds of the present invention was shown in the following Table 68 with a rank of A, B or C. These ranges are as mentioned below.
3 μM≧C>1 μM≧B>0.5 μM≧A

TABLE 68

| Test Compound | IC₅₀ value |
|---|---|
| Example 30 | C |
| Example 45 | C |
| Example 46 | C |
| Example 51 | C |
| Example 59 | C |
| Example 115 | A |
| Example 124 | C |
| Example 136 | A |
| Example 141 | A |
| Example 146 | C |
| Example 152 | B |
| Example 155 | C |
| Example 207 | B |

### Experimental Example 2

Inhibitory Effect on the Rhythmic Bladder Contractions Induced by Substance P in Anesthetized Rats

For the experiments, Sprague-Dawley female rats (9 to 12 weeks old) weighing between 200 to 300 g were used. After urethane anesthetization (subcutaneously administered with a dose of 1.2 g/kg), cannulae were placed in both right and left femoral veins. One intravenous catheter was used for administration of compounds, and the other was for the substance P (0.33 μg/kg/min) infusion. We also cannulated into ureter to pass urine. Polyethylene catheters were inserted into carotid artery for continuous monitoring of arterial blood pressure and heart rate. For continuous infusion, transurethral bladder catheter was inserted into the bladder through the urethra and tied in place by a ligature around the urethral orifice. One end of the catheter was attached to a pressure transducer in order to measure intravesical pressure. The other end of the catheter was used for infusion of saline into the bladder. After stabilization of blood pressure and heart rate and after the bladder was emptied, cystometry was performed by filling the bladder slowly with about 0.6 ml of saline. After about 10 minutes, intravenous infusion of substance P (0.33 μg/kg/min) was started for stabilization of the micturition reflex. Compounds were administered after stable rhythmic bladder contraction was obtained over 15 minutes. All compounds were dissolved or suspended in saline containing 0.5% Tween 80 for intravenous administration (0.1 ml/kg). The rhythmic contraction frequency and the intravesical pressure were observed for 35 minutes after administration of the test compound.

As a result, compounds of the present invention decreased the frequency of bladder rhythmic contraction without changing the amplitude of contraction. Also, we determined a time (minute) during which the frequency of the rhythmic contraction had been completely inhibited by administering 0.25 mg/kg of compound. A 100% inhibition time (minute) of the selected compounds of the present invention is shown in the following Table 69.

TABLE 69

| | 100% inhibiting |
|---|---|
| Test Compound | time (min) |
| Example 59 | 5.7 |
| Example 93 | 9.1 |
| Example 124 | 8.2 |
| Example 135 | 17.8 |
| Example 142 | 17.3 |
| Example 171 | 12.8 |
| Example 207 | 13.9 |

Also, pre-administration of iberiotoxin, a selective large conductance calcium-activated K channel blocker (0.15 mg/kg, intravenous administration) reduced inhibitory effect of the compounds of the present invention on the rhythmic bladder contraction. Thus, it is suggested from the results that the compounds of the present invention have a detrusor relaxing activity through the large conductance calcium-activated K channel.

Thus, it was shown that compounds of the present invention were effective for prophylaxis and treatment of diseases such as pollakiuria, urinary incontinence and the like through the large conductance calcium-activated K channel opening activity.

### INDUSTRIAL APPLICABILITY

The compound or a pharmaceutically acceptable salt which is an active ingredient of the present invention has an excellent large conductance calcium-activated K channel opening activity and hyperpolarizes a membrane electric potential of cells, so that it is useful for a prophylactic, relief and/or treatment for pollakiuria, urinary incontinence, asthma, chronic obstructive pulmonary disease (COPD), and the like.

1. A large conductance calcium-activated K channel opener comprising a compound of the formula (I): 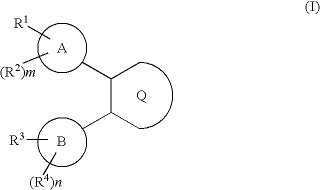
wherein Ring A is benzene or a heterocyclic ring;
Ring B is benzene, a heterocyclic ring, a cycloalkane or a cycloalkene;
Ring Q is a group selected from the following formulae: 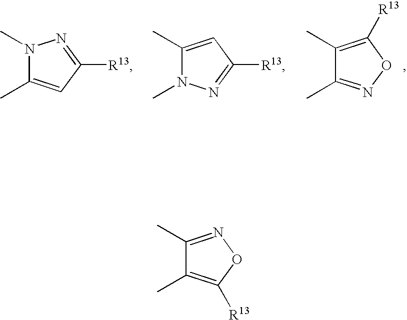
R¹ and R³ may be the same or different from each other, and each is a group selected from the following formulae: 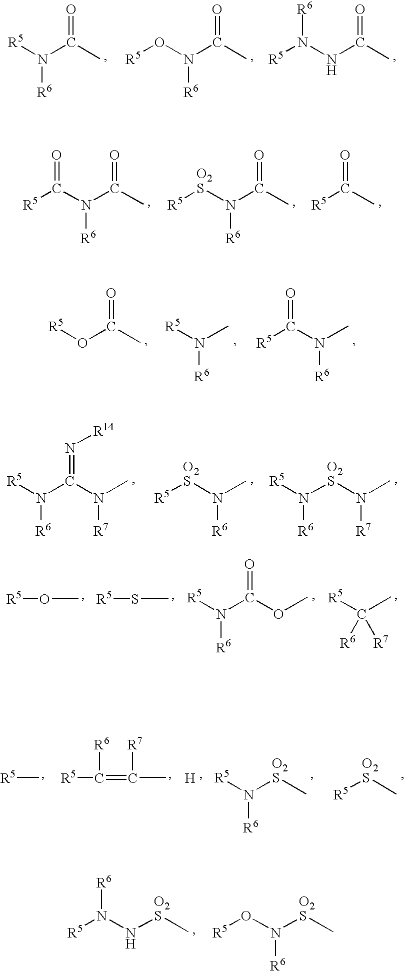
R⁵ and R⁶ may be the Same or different from each other, and each is (1) hydrogen, (2) an optionally substituted alkyl, (3) an optionally substituted cycloalkyl which may be fused with an aryl, (4) an optionally substituted aryl, (5) an optionally substituted heterocyclic group, or (6) an alkoxycarbonyl, or (7) R⁵ and R⁶ may be combined to form an optionally substituted heterocyclic ring in combination with atom(s) to which they are bonded;
R⁷ is (1) hydrogen, (2) an optionally substituted alkyl, (3) an optionally substituted cycloalkyl which may be fused with an aryl, (4) an optionally substituted aryl, or (5) an alkoxycarbonyl;
R¹⁴ is hydrogen, an alkoxy, hydroxyl, cyano or an optionally substituted alkyl;
m and n may be the same or different from each other, and each is 0, 1 or 2;
R² and R⁴ may be the same or different from each other, and each is oxo, cyano, nitro, hydroxyl, an alkoxy, a halogen, carboxy, an alkoxycarbonyl, an optionally substituted carbamoyl, an optionally substituted amino or an optionally substituted alkyl; provided that when m is 2, two R² may be the same or different from each other, and when n is 2, two R⁴ may be the same or different from each other;
or R¹ and R² may be combined to form a group selected from the following formulae with Ring A; 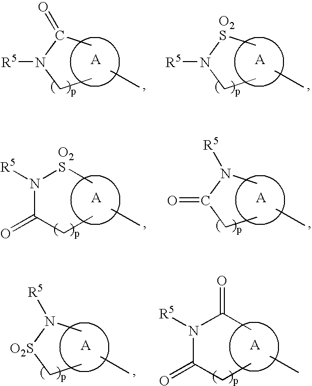
or R³ and R⁴ may be combined to form a group selected from the following formulae with Ring B; 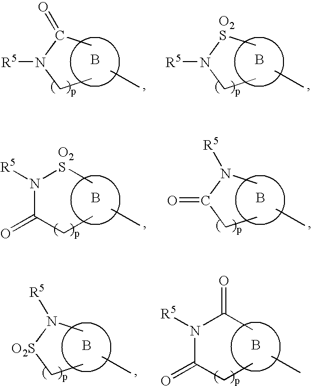
p is an integer of 1 to 3; and
R¹³ is (1) an optionally substituted alkyl, (2) cyano, (3) hydrogen, (4) a halogen, (5) an optionally substituted amino, (6) an alkenyl, (7) an optionally substituted carbamoyl, (8) an alkoxycarbonyl, (9) carboxy, (10) a heterocyclic group, (11) hydroxyl or (12) an alkoxy,
or a pharmaceutically acceptable salt thereof as an active ingredient.

2. The large conductance calcium-activated K channel opener according to claim 1, wherein the substituent(s) for the optionally substituted alkyl of R⁵, R⁶ and R⁷ are 1 to 7 independently selected halogen(s) and/or 1 to 3 groups selected from the following groups: 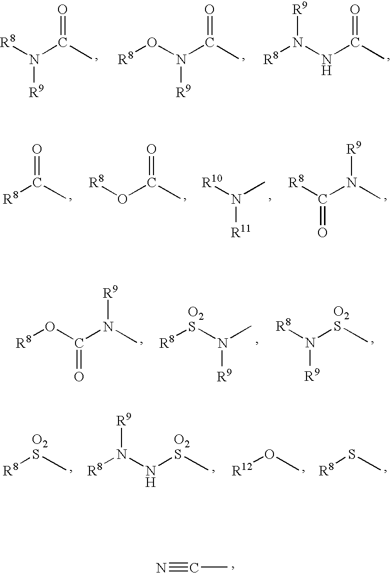
an optionally substituted heterocyclic group and an optionally substituted aryl,
wherein R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxy-alkyl, (5) an alkoxycarbonyl, (6) an optionally substituted heterocyclic group or (7) an optionally substituted aryl, or (8) R⁸ and R⁹ may be combined to form an optionally substituted heterocyclic ring in combination with atom(s) to which they are bonded; R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group; R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group.

3. The large conductance calcium-activated K channel opener according to claim 1, wherein
Ring B is benzene, a heterocyclic ring, a cycloalkane or a cycloalkene,
R¹ is a group selected from the following formulae: 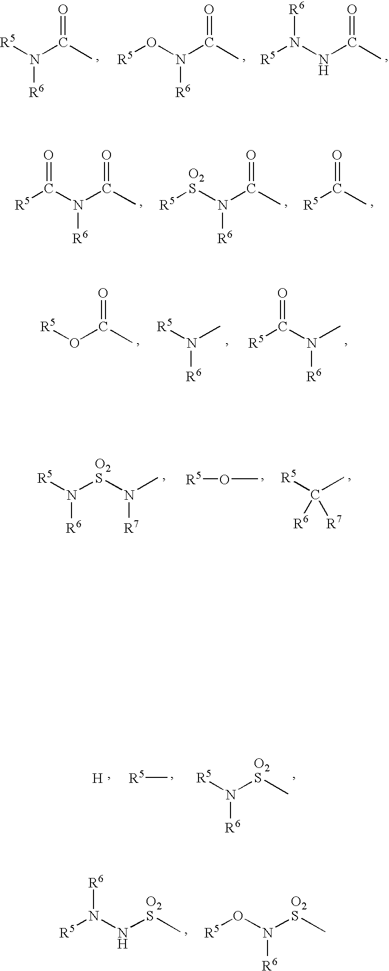
R³ is a group selected from the following formulae: 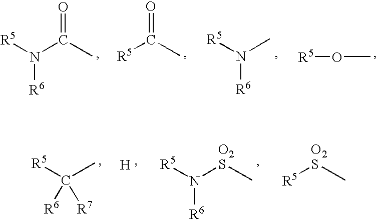
R⁵ is (1) hydrogen, (2) an alkyl which may be substituted by 1 to 7 independently selected halogen(s) and/or by 1 to 3 groups selected from the following groups: 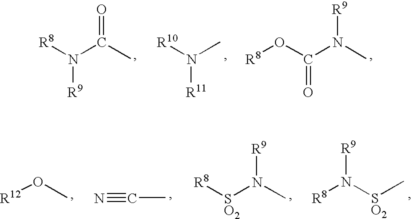
an optionally substituted heterocyclic group and an optionally substituted aryl,
(3) an optionally substituted cycloalkyl which may be fused with an aryl, (4) an optionally substituted aryl, or (5) an optionally substituted heterocyclic group;
R⁶ is hydrogen, an alkyl or an alkoxycarbonyl, or R⁵ and R⁶ may be combined to form an optionally substituted heterocyclic ring in combination with atoms to which they are bonded;
R⁷ is hydrogen, an alkyl or an alkoxycarbonyl;
R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, (6) an optionally substituted aryl, or (7) R⁸ and R⁹ may be combined to form an optionally substituted heterocyclic ring in combination with atom(s) to which they are bonded;
R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group;
R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
m and n may be the same or different from each other, and each is 0, 1 or 2; and
R² and R⁴ may be the same or different from each other, and each is oxo, cyano, nitro, hydroxyl, an alkoxy, a halogen or an optionally substituted alkyl.

4. The large conductance calcium-activated K channel opener according to claim 1, wherein
Ring B is (1) benzene or (2) a heterocyclic ring selected from thiophene, pyridine, pyrimidine, pyrazine, benzothiophene, 2,3-dihydroindole, 2,3-dihydrobenzofuran and 1,4-benzodioxane or (3) a cyclohexene;
R¹ is a group selected from the following formulae: 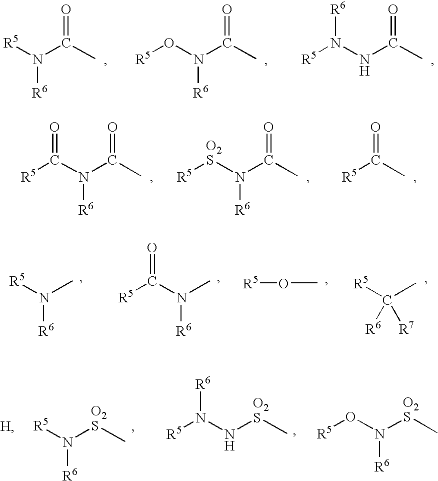
R³ is a group selected from the following formulae: 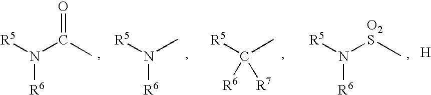
R⁵ is (1) hydrogen, (2) an alkyl which may be substituted by 1 to 7 independently selected halogen(s) and/or by 1 or 2 groups selected from the following groups: 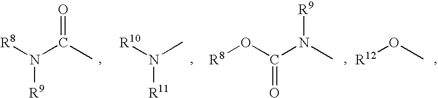
an optionally substituted heterocyclic group and an optionally substituted aryl,
(3) an optionally substituted cycloalkyl which may be fused with an aryl, (4) an optionally substituted aryl, or (5) an optionally substituted heterocyclic group;
R⁶ is hydrogen or an alkyl, or R⁵ and R⁶ may be combined to form a heterocyclic ring which may be substituted by hydroxyalkyl, in combination with atom(s) to which they are bonded;
R⁷ is hydrogen or an alkyl;
R⁸ and R⁹ may be the same or different from each other, and each is (1) hydrogen, (2) an alkyl which may be substituted by an optionally substituted aryl or by an optionally substituted heterocyclic group, (3) hydroxyalkyl or (4) an alkoxyalkyl;
R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
m and n may be the same or different from each other, and each is 0, 1 or 2;
R² and R⁴ may be the same or different from each other, and each is oxo, cyano, nitro, hydroxyl group, an alkoxy, a halogen or an alkyl which may be substituted by hydroxyl group; and
R¹³ is (1) hydrogen, (2) an alkyl which may be substituted by a group selected from a halogen, hydroxyl group, an optionally substituted alkoxy, cyano, carboxy, carbamoyl, an alkoxycarbonyl, an optionally substituted amino and an optionally substituted imino, (3) an alkenyl, or (4) a heterocyclic group.

5. The large conductance calcium-activated K channel opener according to claim 1, wherein
Ring A is benzene, thiophene, pyridine or pyrazole;
Ring B is (1) benzene, (2) a heterocyclic ring selected from thiophene, pyridine, pyrimidine, pyrazine, benzothiophene, 2,3-dihydroindole and 1,4-benzodioxane, or (3) a cyclohexene;
R¹ is a group selected from the following formulae: 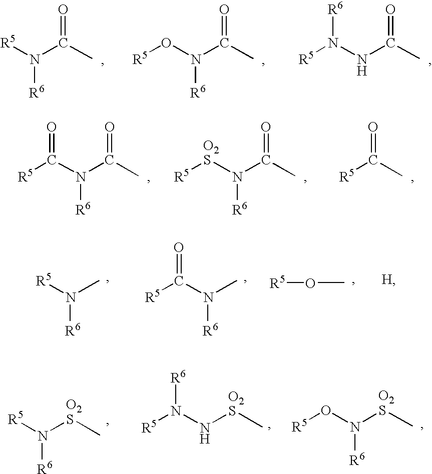
R³ is a group selected from the following formulae: 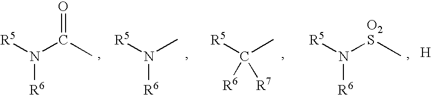
R⁵ is (1) hydrogen, (2) an alkyl which may be substituted by 1 to 7 independently selected halogen(s) and/or by 1 or 2 groups selected from the following groups: 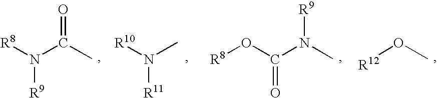
an optionally substituted heterocyclic group and an optionally substituted aryl,
(3) a cycloalkyl fused with an aryl which may be substituted by hydroxyl(s), or (4) a heterocyclic group;
R⁶ is hydrogen or an alkyl, or R⁵ and R⁶ may be combined to form a heterocyclic ring which may be substituted by hydroxyalkyl;
R⁷ is hydrogen or an alkyl;
R⁸, R⁹, R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, or (6) an optionally substituted aryl;
R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
m and n may be the same or different from each other, and each is 0, 1 or 2;
R² and R⁴ may be the same or different from each other, and each is cyano, nitro, hydroxyl, a halogen, an alkyl or an alkoxy; and
R¹³ is (1) hydrogen, (2) an alkyl which may be substituted by group(s) selected from a halogen, hydroxyl, an alkoxy which may be substituted by group(s) selected from a halogen and phenyl, cyano, carboxy, carbamoyl, an alkoxycarbonyl, an amino which may be substituted by phenyl, and an imino which may be substituted by group(s) selected from an alkoxy and hydroxyl, (3) an alkenyl or (4) 4,5-dihydroxazol-2-yl.

6. The large conductance calcium-activated K channel opener according to claim 1, wherein
R¹ is a group selected from the following formulae:

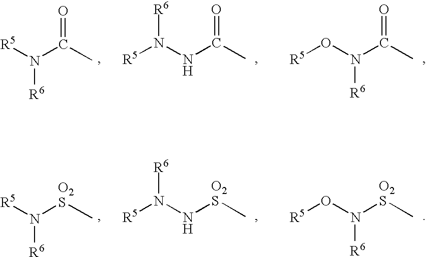

7. A compound of the formula (Ia): 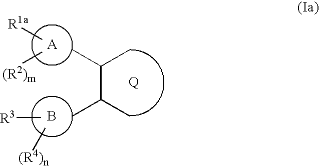
wherein Ring A is benzene or a heterocyclic ring;
Ring B is benzene, a heterocyclic ring, a cycloalkane or a cycloalkene;
Ring Q is a group selected from the following formulae: 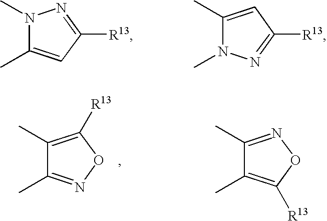
R^{1a} is a group selected from the following formulae: 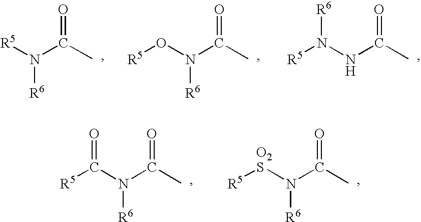
R³ is a group selected from the following formulae: 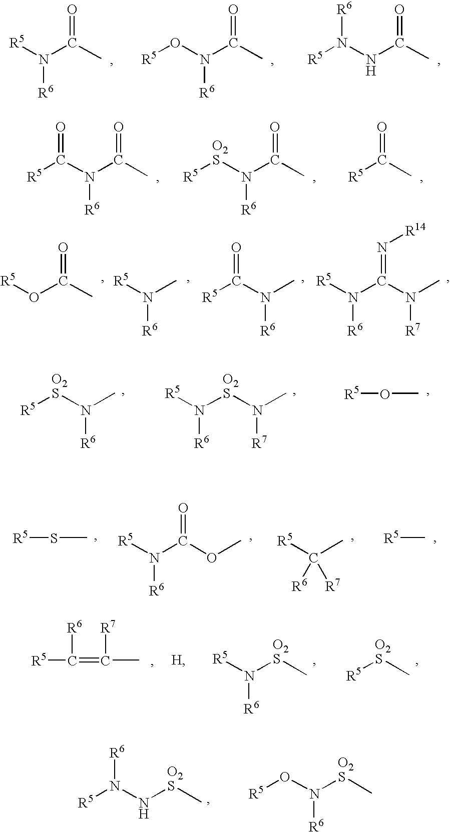
R⁵ and R⁶ may be the same or different from each other, and each is (1) hydrogen, (2) an optionally substituted alkyl, (3) an optionally substituted cycloalkyl which may be fused with an aryl, (4) an optionally substituted aryl, (5) an optionally substituted heterocyclic group, or (6) an alkoxycarbonyl, or (7) R⁵ and R⁶ may be combined to form an optionally substituted heterocyclic ring in combination with atom(s) to which they are bonded;
R⁷ is (1) hydrogen, (2) an optionally substituted alkyl, (3) an optionally substituted cycloalkyl which may be fused with an aryl, (4) an optionally substituted aryl, or (5) an alkoxycarbonyl;
R¹⁴ is hydrogen, an alkoxy, hydroxyl, cyano or an optionally substituted alkyl;
m and n may be the same or different from each other, and each is 0, 1 or 2;
R² and R⁴ may be the same or different from each other, and each is oxo, cyano, nitro, hydroxyl, an alkoxy, a halogen, carboxy, an alkoxycarbonyl, an optionally substituted carbamoyl, an optionally substituted amino or an optionally substituted alkyl; provided that when m is 2, two R² may be the same or different from each other, and when n is 2, two R⁴ may be the same or different from each other;
or R^{1a} and R² may be combined to form a group of the following formula with Ring A: 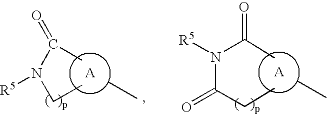
or R³ and R⁴ may be combined to form a group selected from the following formulae with Ring B: 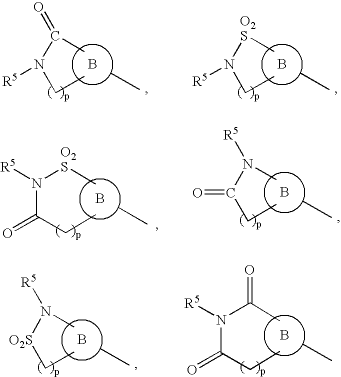
p is an integer of 1 to 3; and
R¹³ is (1) an optionally substituted alkyl, (2) cyano, (3) hydrogen, (4) a halogen, (5) an optionally substituted amino, (6) an alkenyl, (7) an optionally substituted carbamoyl, (8) an alkoxycarbonyl, (9) carboxy, (10) a heterocyclic group, (11) hydroxyl or (12) an alkoxy;
provided that (i) the compound in which Ring A and Ring B are benzenes;
Ring Q is 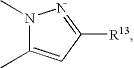
R³ is hydroxyl, an alkoxy or a cycloalkyloxy which are substituted at 2-position,
R⁴ is methoxy substituted at 6-position, and
R¹³ is an alkoxycarbonyl or carboxy,
(ii) N-(3-isopropoxypropyl)-4-(3-methyl-5-phenyl-1H-pyrazol-1-yl)benzamide,
(iii) 4-(1-(4-aminosulfonylphenyl)-3-difluoromethyl-1H-pyrazol-5-yl)benzamide, and
(iv) 4-[5-(4-chlorophenyl)-3-(3-hydroxypropyl)-1H-pyrazol-1-yl]-N-methylbenzohydroxamic acid
are excluded,
or a pharmaceutically acceptable salt thereof.

8. The compound or a pharmaceutically acceptable salt thereof according to claim 7, wherein the substituent(s) for the optionally substituted alkyl of R⁵, R⁶ and R⁷ are 1 to 7 independently selected halogen(s) and/or 1 to 3 groups selected from the following groups: 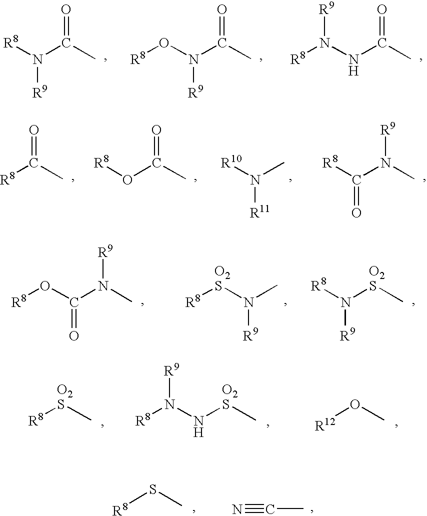
an optionally substituted heterocyclic group and an optionally substituted aryl,
wherein R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkoxycarbonyl, (6) an optionally substituted heterocyclic group or (7) an optionally substituted aryl, or (8) R⁸ and R⁹ may be combined to form an optionally substituted heterocyclic ring in combination with atom(s) to which they are bonded; R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group; R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group.

9. The compound or a pharmaceutically acceptable salt thereof according to claim 7, wherein
Ring B is benzene, a heterocyclic ring or a cycloalkane;
R^{1a} is a group selected from the following formulae: 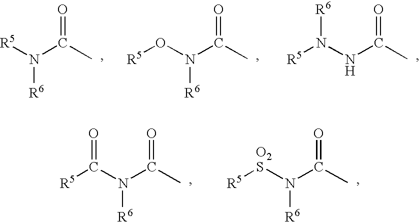
R³ is a group selected from the following formulae: 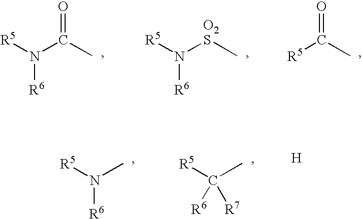
R⁵ is (1) hydrogen, (2) an alkyl which may be substituted by 1 to 7 independently selected halogen(s) and/or by 1 to 3 groups selected from the following groups: 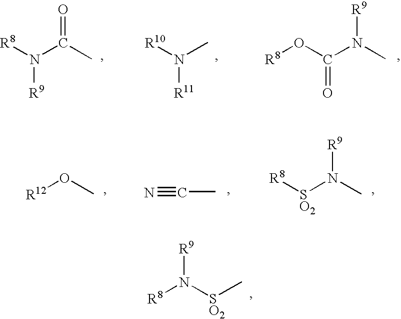
an optionally substituted heterocyclic group and an optionally substituted aryl,
(3) an optionally substituted cycloalkyl which may be fused with an aryl, (4) an optionally substituted aryl, or (5) an optionally substituted heterocyclic group;
R⁶ is hydrogen or an alkyl, or R⁵ and R⁶ may be combined to form an optionally substituted heterocyclic ring in combination with atom(s) to which they are bonded;
R⁷ is hydrogen, an alkyl or an alkoxycarbonyl;
R⁸ and R⁹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, (6) an optionally substituted aryl, or (7) R⁸ and R⁹ may be combined to form an optionally substituted heterocyclic ring in combination with atom(s) to which they are bonded;
R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group;
R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
m and n may be the same or different from each other, and each is 0, 1 or 2; and
R² and R⁴ may be the same or different from each other, and each is oxo, cyano, nitro, hydroxyl, an alkoxy, a halogen or an optionally substituted alkyl.

10. The compound or a pharmaceutically acceptable salt thereof according to claim 7, wherein
Ring B is (1) benzene or (2) a heterocyclic ring selected from thiophene, pyridine, pyrimidine, pyrazine, benzothiophene, 2,3-dihydroindole, 2,3-dihydrobenzofuran and 1,4-benzodioxane;
R^{1a} is a group selected from the following formulae: 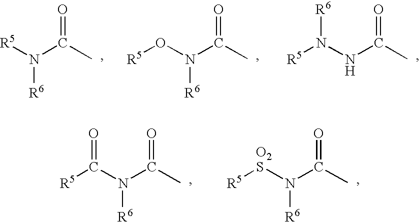
R³ is a group selected from the following formulae: 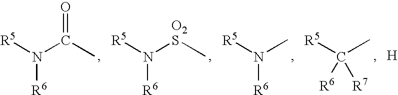
R⁵ is (1) hydrogen, (2) an alkyl which may be substituted by 1 to 7 independently selected halogen(s) and/or by 1 or 2 groups selected from the following groups: 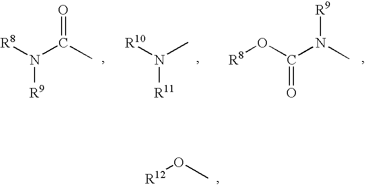
an optionally substituted heterocyclic group and an optionally substituted aryl,
(3) an optionally substituted cycloalkyl which may be fused with an aryl, (4) an optionally substituted aryl, or (5) an optionally substituted heterocyclic group;
R⁶ is hydrogen or an alkyl, or R⁵ and R⁶ may be combined to form a heterocyclic ring which may be substituted by a hydroxyalkyl, in combination with atom(s) to which they are bonded;
R⁷ is hydrogen or an alkyl;
R⁸ and R⁹ may be the same or different from each other, and each is (1) hydrogen, (2) an alkyl which may be substituted by an optionally substituted aryl or by an optionally substituted heterocyclic group, (3) a hydroxyalkyl or (4) an alkoxyalkyl;
R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
m and n may be the same or different from each other, and each is 0, 1 or 2;
R² and R⁴ may be the same or different from each other, and each is oxo, cyano, nitro, hydroxyl, an alkoxy, a halogen or an alkyl which may be substituted by hydroxyl(s); and
R¹³ is (1) hydrogen, (2) an alkyl which may be substituted by group(s) selected from a halogen, hydroxyl, an optionally substituted alkoxy, cyano, carboxy, an optionally substituted amino and an optionally substituted imino, (3) an alkenyl, or (4) a heterocyclic group.

11. The compound or a pharmaceutically acceptable salt thereof according to claim 7, wherein
Ring A is benzene, thiophene, pyridine or pyrazole;
Ring B is (1) benzene, or (2) a heterocyclic ring selected from thiophene, pyridine, pyrimidine, pyrazine, benzothiophene and 1,4-benzodioxane;
R^{1a} is a group selected from the following formulae: 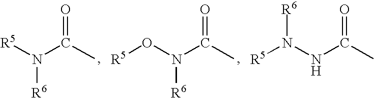
R³ is a group selected from the following formulae: 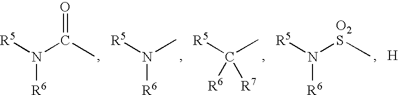
R⁵ is (1) hydrogen, (2) an alkyl which may be substituted by 1 to 7 independently selected halogen(s) and/or by 1 or 2 groups selected from the following groups: 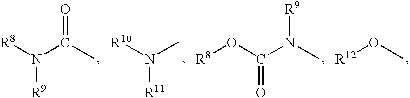
an optionally substituted heterocyclic group and an optionally substituted aryl,
(3) a cycloalkyl fused with an aryl which may be substituted by hydroxyl, or (4) a heterocyclic group;
R⁶ is hydrogen or an alkyl, or R⁵ and R⁶ may be combined to form a heterocyclic ring which may be substituted by hydroxyalkyl;
R⁷ is hydrogen or an alkyl;
R⁸, R⁹, R¹⁰ and R¹¹ may be the same or different from each other, and each is (1) hydrogen, (2) an alkyl which may be substituted by an optionally substituted aryl or by an optionally substituted heterocyclic group, (3) a hydroxyalkyl, (4) an alkoxyalkyl, (5) an optionally substituted heterocyclic group, or (6) an optionally substituted aryl;
R¹² is (1) hydrogen, (2) an alkyl which may be substituted by an optionally substituted aryl or by an optionally substituted heterocyclic group, (3) a hydroxyalkyl, (4) an alkoxyalkyl or (5) an optionally substituted heterocyclic group;
m and n may be the same or different from each other, and each is 0, 1 or 2;
R² and R⁴ may be the same or different from each other, and each is cyano, nitro, hydroxyl, a halogen, an alkyl or an alkoxy; and
R¹³ is (1) hydrogen, (2) an alkyl which may be substituted by group(s) selected from a halogen, hydroxyl, an alkoxy which may be substituted by group(s) selected from a halogen and phenyl, cyano, carboxy, carbamoyl, an alkoxycarbonyl, an amino which may be substituted by phenyl, and an imino which may be substituted by group(s) selected from an alkoxy and hydroxyl, (3) an alkenyl or (4) 4,5-dihydroxazol-2-yl.

12. A medicine comprising the compound or a pharmaceutically acceptable salt thereof according to claim 7.

13. The medicine according to claim 12, which is a large conductance calcium-activated K channel opener.

14. The large conductance calcium-activated K channel opener according to claim 1, which is for the prophylaxis and/or treatment of pollakiuria, urinary incontinence, asthma or chronic obstructive pulmonary diseases.
